(19) United States
(12) Patent Application Publication
(10) Pub. No.: US 2024/0369551 A1
(43) Pub. Date: Nov. 7, 2024

(54) NANOSENSOR FOR DETECTING SARS-CoV-2 SPECIFIC ANTIBODIES

(71) Applicant: Agency for Science, Technology and Research, Singapore (SG)

(72) Inventors: Tedrick Thomas Salim Lew, Singapore (SG); Sian Yang Ow, Singapore (SG); Xiaodi Su, Singapore (SG); Laura Sutarlie, Singapore (SG); Khin Moh Moh Aung, Singapore (SG); Lisa Fong Poh Ng, Singapore (SG)

(21) Appl. No.: 18/284,618

(22) PCT Filed: Mar. 29, 2022
(86) PCT No.: PCT/SG2022/050174

§ 371 Date: Sep. 28, 2023

(30) Foreign Application Priority Data
Mar. 30, 2021 (SG) 10202103271S

(51) Int. Cl. G01N 33/553; G01N 33/543; G01N 33/569

(52) U.S. Cl. CPC G01N 33/553; G01N 33/54346; G01N 33/56983; G01N 2333/165; G01N 2469/20

(57) ABSTRACT

The present invention relates to a nanosensor for detecting SARS-CoV-2 specific antibodies, wherein the nanosensor comprises a metal nanoparticle functionalised with one or more B-cell linear peptide epitopes derived from the spike (S) protein or nucleocapsid (N) protein of SARS-CoV-2. In some preferred embodiments, the metal nanoparticle comprises gold, silver, or a combination of gold and silver. The present invention also relates to various methods involving the use of the nanosensor.

### TECHNICAL FIELD

The present disclosure relates broadly to a nanosensor for detecting the presence of SARS-CoV-2 specific antibodies. Also provided is an assay comprising a plurality of the nanosensors, and various methods involving the use of the nanosensor.

### BACKGROUND

The ongoing pandemic caused by SARS-CoV-2 underscores the need for rapid, accurate, and cost-effective diagnostic assays to contain the spread of COVID-19 at national and global scales. In addition to molecular and antigen tests, serological assays play a key role in enabling timely management and effective response to combat pandemic outbreaks. (1) Such tests detect antibodies produced due to SARS-CoV-2 infection, which are detectable as early as 2 days after symptom onset in some patients. (2,3) Since antibodies persist much longer than viral RNA or antigens in biological fluids, their presence can inform current exposure, disease progression, and past infection or immunity. (4) As such, serological testing has been crucial in epidemiological surveillance to identify SARS-CoV-2-infected individuals who may not have been diagnosed due to asymptomatic infection or mild illness, providing important information to better understand COVID-19 transmission dynamics. (5,6) Immunological assays can also complement antigen or molecular tests particularly at the later stage of infection, where viral load is reduced significantly upon shedding and seroconversion. (7-9) Additionally, as doses of COVID-19 vaccines have been administered in many countries, serology assays can be a key tool to evaluate vaccine efficiency and humoral response kinetics postvaccination. (10-12) To date, most serological tests to detect SARS-CoV-2-specific antibodies are based on enzyme-linked immunoabsorbent assays (ELISA) or chemiluminescent assays (CLIA) employing recombinant coronavirus proteins. (13,14). However, in terms of assay formats, classical immunoassays are labor-intensive and require multiple reagents with long incubation, washing, and reaction steps (2-5 h). (22) Thus, the development of a cost-effective serological assay that can provide simple, fast, and specific determination of SARS-CoV-2 antibodies with minimal equipment remains an unmet need in immunological diagnosis.

### SUMMARY

In one aspect, there is provided a nanosensor for detecting SARS-CoV-2 antibodies, comprising a metal nanoparticle functionalized with one or more B-cell linear peptide epitopes derived from the spike (S) protein or nucleocapsid (N) protein of SARS-CoV-2.

In one embodiment, the one or more epitopes are conjugated to the nanoparticle surface either:
a) via a biotin-streptavidin interaction, or
b) via a thiol-metal interaction wherein the nanoparticle is coated with a spacer comprising at least 4 amino acids and at least one cysteine residue.

In one embodiment, the one or more epitopes are conjugated via a biotin-streptavidin interaction, for example wherein the metal nanoparticle is coated with streptavidin (SA) and the epitopes are biotinylated.

In one embodiment, the one or more epitopes are conjugated via a thiol-metal interaction, for example wherein the metal nanoparticle is coated with a thiolyated spacer and the epitopes are thiolyated.

In one embodiment, the spacer comprises the amino acid sequence CALNN.

In one embodiment, the metal is selected from the group consisting of gold, silver, and a combination of gold and silver, such as Au₅₀Ag₅₀. Advantageously, the present inventors have demonstrated that metal nanoparticles comprising these metals are particularly suitable for use in the disclosed nanosensors.

In one embodiment, the metal nanoparticle is a gold nanoparticle (AuNP).

In one embodiment, the epitope is derived from the spike (S) protein.

In one embodiment, the epitope is derived from the S1 or S2 subunit of the spike protein.

In one embodiment, the epitope is derived from the S2 subunit of the spike protein.

In one embodiment, the epitope comprises an amino acid sequence selected from the group consisting of:

(S14P5)
(SEQ ID NO: 3)
TESNKKFLPFQQFGRDIA,
(S20P2)
(SEQ ID NO: 4)
GIAVEQDKNTQEVFAQVK,
(S21P2)
(SEQ ID NO: 5)
PSKPSKRSFIEDLLFNKV,
(N4P5)
(SEQ ID NO: 6)
NNAAIVLQLPQGTTLPKG,

and
an amino acid sequence at least 95% identical to one of the above.

In one embodiment, the epitope comprises an amino acid sequence selected from the group consisting of:

(S14P5)
(SEQ ID NO: 3)
TESNKKFLPFQQFGRDIA,
(S20P2)
(SEQ ID NO: 4)
GIAVEQDKNTQEVFAQVK,
(S21P2)
(SEQ ID NO: 5)
PSKPSKRSFIEDLLFNKV,

and
an amino acid sequence at least 95% identical to one of the above.

In one embodiment, the epitope comprises an amino acid sequence selected from the group consisting of:

(S14P5)
(SEQ ID NO: 3)
TESNKKFLPFQQFGRDIA,
(S21P2)
(SEQ ID NO: 5)
PSKPSKRSFIEDLLFNKV,

and
an amino acid sequence at least 95% identical to one of the above.

In one embodiment, the epitope comprises the amino acid sequence TESNKKFLPFQQFGRDIA (S14P5) or an amino acid sequence at least 95% identical thereto.

In one embodiment, the nanoparticle is functionalized with at least two different epitopes, such as 2 to 20 different epitopes, for example 2, 3, 4, 5, 6, 7, 8, 9, 10, 11, 12, 13, 14, 15, 16, 17, 18, 19 or 20 different epitopes.

In one embodiment, the nanoparticle is functionalized with two different epitopes.

In one embodiment, at least one epitope is derived from the S1 subunit of the spike protein, and at least one other epitope is derived from the S2 subunit of the spike protein.

In one embodiment, the two different epitopes are TESNKKFLPFQQFGRDIA (S14P5) (SEQ ID NO: 3) or an amino acid sequence at least 95% identical thereto, and PSKPSKRSFIEDLLFNKV (S21P2) (SEQ ID NO: 5) or an amino acid sequence at least 95% identical thereto.

In one embodiment, the two different epitopes are TESNKKFLPFQQFGRDIA (S14P5) or an amino acid sequence at least 95% identical thereto, and GIAVEQDKNTQEVFAQVK (S20P2) (SEQ ID NO: 4) or an amino acid sequence at least 95% identical thereto.

In one embodiment, the nanoparticle is passivated with a substance suitable for passivating the nanoparticle against biofluids (such as serum, blood, or plasma), for example by coating the nanoparticle with a polymer layer

In one embodiment, the nanoparticle is passivated by coating the nanoparticle with polyethylene glycol (PEG).

In one aspect, there is provided an in vitro assay for detecting the presence of SARS-CoV-2 specific antibodies in a sample, comprising a plurality of nanosensors as defined above.

In one embodiment, the assay further comprises one or more of the following: a stabilizer (such as Tween 20), a buffer (such as PBS), a control antibody such as a SARS-CoV-2 antibody, a SARS-CoV antibody or a normal human IgG, a microplate, and combinations thereof.

In one aspect, there is provided an in vitro method of detecting the presence of SARS-CoV-2 specific antibodies in a sample, comprising contacting the sample with a plurality of nanosensors as defined above, and detecting the degree of nanoparticle aggregation.

In one aspect, there is provided an in vitro method of diagnosing a subject as having a SARS-CoV-2 infection, comprising:
detecting the presence of SARS-CoV-2 specific antibodies in a sample from the subject by contacting the sample with a plurality of nanosensors as defined above,
detecting the degree of nanoparticle aggregation in the sample,
comparing the degree of nanoparticle aggregation in the sample to the degree of nanoparticle aggregation in a control sample from a subject that does not have a SARS-CoV-2 infection,
determining if the degree of nanoparticle aggregation is higher in the sample compared to the degree of nanoparticle aggregation in the control sample, thereby diagnosing the subject as having a SARS-CoV-2 infection.

In one aspect, there is provided a method of treating a subject identified as having a SARS-CoV-2 infection, comprising:
detecting the presence of SARS-CoV-2 specific antibodies in a sample from the subject by contacting the sample with a plurality of nanosensors as defined above,
detecting the degree of nanoparticle aggregation in the sample,
comparing the degree of nanoparticle aggregation in the sample to the degree of nanoparticle aggregation in a control sample from a subject that does not have a SARS-CoV-2 infection,
determining if the degree of nanoparticle aggregation is higher in the sample compared to the degree of nanoparticle aggregation in the control sample, thereby identifying the subject as having a SARS-CoV-2 infection; and
administering a therapeutic agent, such as an anti-viral agent to the subject.

In one embodiment, the therapeutic agent is selected from the group consisting of Paxlovid™ (nirmatrelvir and ritonavir), molnupiravir, fluvoxamine, sotrovimab, bebtelovimab, remdesivir and a combination thereof.

In one embodiment, the degree of nanoparticle aggregation is proportional to the quantity of SARS-CoV-2 specific antibodies in the sample.

In one embodiment, the range of SARS-CoV-2 specific antibodies that can be detected in the sample is about 3-1000 nM, such as about 3.2-1000 nM.

In one embodiment, the nanoparticle aggregation results in a visible colour change, for example from red to purple.

In one embodiment, the degree of nanoparticle aggregation in a sample from a subject having a SARS-CoV-2 infection is more than the mean+3 standard deviations of the degree of nanoparticle aggregation in a control sample.

In one embodiment, the degree of nanoparticle aggregation is detected by determining the % change in A_{534 nm}, for example by measuring the absorbance using a spectrophotometer or microplate reader.

In one embodiment, a % change in A₅₃₄ nm of about 15 to 25% or more, such as 20% or more indicates that the sample is from a subject having a SARS-CoV-2 infection.

In one embodiment, the sample is plasma or serum, in particular plasma.

### Definitions

The term “nanosensor” as used herein refers to a nanoscale-based device that measures a physical quality and produces a signal that can be detected and analysed.

There are different types of nanosensors for various applications, most notably, in medicine, defence and environmental applications. All nanosensors share the same basic workflow: the nanosensor binds selectively to an analyte, produces a detectable signal based on the interaction of the sensor with the analyte, which can then be processed into useful metrics. Within the context of the present disclosure, the term “nanosensor” generally refers to a or a “nanobiosensor” or “biological nanosensor”.

The term “biosensor” as used herein refers to a device designed to detect or quantify a biochemical molecule, such as a nucleic acid sequence, such as DNA, or a protein, polypeptide or peptide.

Hence, the term “nanobiosensor” used interchangeably with “biological nanosensor” as used herein refers to a nanoscale device that binds selectively to a biochemical molecule and based on that interaction, produces a signal which can be detected and analysed.

The term “nanoparticle” abbreviated as “NP” as used herein refers to a particle of any shape with dimensions between 1 and 1000 nm in diameter, although nanoparticles are most commonly in the size range of 1-100 nm. The nanoparticle of the present disclosure may be spherical, substantially spherical, or non-spherical, such as irregularly shaped particles or ellipsoidally shaped particle. Nanoparticles are broadly divided into various categories depending on their morphology, size and chemical properties. Some of the more well-known classes of nanoparticles include carbon-based nanoparticles, metal nanoparticles, ceramic nanoparticles, semiconductor nanoparticles, polymeric nanoparticles, and lipid-based nanoparticles. Nanoparticles have many unique properties due to their large surface area to mass ratio and small size which allow them to have interaction at the molecular level. Many of these properties, such as their stability, solubility, chemical and/or biological activity can be altered by coating them with various substances, which is a process called functionalization.

The term “metal nanoparticle” as used herein refers to a nanoparticle which is made of a pure metal such as gold, platinum, silver, titanium, zinc, cerium, iron, and thallium, a combination of pure metals (such as an alloy of gold and silver), or compounds thereof, for example oxides, hydroxides, sulfides, phosphates, fluorides, and chlorides. Within the context of the present disclosure, the term “metal nanoparticle” generally refers to a nanoparticle which is made of a pure metal or a combination of pure metals.

The term “nanoparticle aggregation” as used herein refers to the formation and growth of nanoparticle clusters in aqueous dispersions. Such aggregates or clusters comprise a tightly bound collection of nanoparticles which are difficult to break up into primary particles using mechanical forces. For many types of nanoparticles, spectral peaks can shift due to changes in the aggregation state of the nanoparticles. “Blue-shifting” refers to an electromagnetic response that is shifted towards shorter wavelengths (i.e. higher frequencies, higher energies). In comparison, Red-shifting refers to a peak that shifts to longer wavelengths. The aggregation of gold nanoparticles for example typically results in a red-shift.

The term “functionalization” as used herein refers to the process of adding new functions, features, or properties to a material by altering the surface chemistry of the material. Thus, when used in the context of nanoparticles, “functionalization” generally refers to the modification of the nanoparticle surface by conjugating chemicals or biomolecules to the surface. There are a variety of functionalization approaches including non-covalent binding, covalent conjugation, amorphous nanoparticle coating and surface epitaxial growth. The skilled person will be aware of a range of suitable approaches for functionalizing nanoparticles.

The term “passivation” as used herein refers to the coating of a material so that it becomes ‘passive’, i.e. less easily affected by the surrounding environment. Thus, a nanoparticle that is ‘passivated’ is a nanoparticle whose surface has been coated with a passivation agent. In the context of the present disclosure, the surrounding environment of the nanoparticle is in general a biofluid. Hence, the passivation agent is one which is suitable for passivating the nanoparticle against biofluids.

The term “peptide” as used herein broadly refers to any chain of amino acid residues connected via peptide bonds. The peptide may be naturally occurring or synthetic (e.g., generated by chemical synthesis or recombinant DNA technology). No particular size is implied by the term “peptide”. In some examples, a peptide may not include a whole virus or a full-length antigen.

The term “epitope” or “antigenic determinant” as used herein refers to a part of an antigen that is recognised by cells of the immune system, in particular recognised by antibodies, B-cells or T-cells. The epitope is the part of the antigen to which an antibody binds. Epitopes are commonly classified as conformation epitopes or linear epitopes depending on their structure and interaction with the target antibody.

The term “linear epitope” or “linear peptide epitope” as used herein refers to an epitope that is recognised by immune cells by its linear sequence of amino acids, i.e. by its primary structure. In contrast, a ‘conformational epitope” is recognised by its specific 3d shape and structure.

The term “B-cell epitope” as used herein refers to the part of an antigen that binds to an antibody or immunoglobulin molecule. Hence, the term “B-cell linear peptide epitope” as used herein refers to an epitope that is recognised by an antibody or immunoglobulin molecule by its linear sequence of amino acids.

The term “SARS-CoV-2” as used herein refers to the coronavirus responsible for novel coronavirus disease 2019 (COVID-19). SARS-CoV-2 is a single-stranded RNA-enveloped virus. Its entire genome is 29,881 bp in length, encoding 9860 amino acids.

The term “large spike glycoprotein” used interchangeably with “spike protein” and “surface glycoprotein”, abbreviated as “S protein” as used herein refers to the spike protein of SARS-CoV-2. The S protein is a 180-200 kDa trimeric class I fusion protein that consists of two subunits, called the S1 and S2 subunits. The S1 subunit (symbol: CoV_S1, Pfam: PF01600, InterPro: IPR002551) is composed of 672 amino acids (residues 14-685 of the S protein) and is responsible for mediating binding to host cells via interactions with the human receptor angiotensin converting enzyme 2 (ACE2). The S2 (symbol: CoV_S2, Pfam: PF01601, InterPro: IPR002552) subunit is composed of 588 amino acids (residues 686-1273) and mediates viral cell membrane fusion by forming a six-helical bundle via the two-heptad repeat domain. The S protein therefore plays an important role in host cell binding and entry.

The term “nucleocapsid protein”, abbreviated as “N protein” as used herein refers to the nucleocapsid protein of SARS-CoV-2 (Symbol: CoV-nucleocap, Pfam: PF00937, Interpof: IPR001218, Uniprot: PODTC9). The Nucleocapsid protein plays a vital role in the transcription and replication of the SARS-CoV-2 virus.

The full amino acid sequence of the S protein, S1 and S2 subunits and N protein is available from the GenBank entry of SARS-CoV-2, GenBank no. MN908947.3. The S protein amino acid sequence (SEQ ID NO: 1) is given in GenBank no. QHD43416.1. The N protein amino acid sequence (SEQ ID NO: 2) is given in GenBank no. QHD43423.2.

The term “biotin-streptavidin interaction” used interchangeably with “streptavidin-biotin interaction” as used herein refers to the non-covalent interaction between biotin and streptavidin. Streptavidin is a 66 kDa protein purified from the bacterium Streptomyces avidinii. Streptavidin homo-tetramers have an extremely high affinity (Kd of the order ˜10⁻¹⁴ ml/L) for biotin (also known as vitamin B7 or vitamin H) and is considered one of the strongest non-covalent interactions found in nature. Additionally, Biotin-binding to streptavidin and avidin is resistant to extremes of heat, pH and proteolysis, making capture of biotinylated molecules possible in a wide variety of environments.

The term “biotinylation” as used herein refers to the process of covalently attaching biotin to proteins, such as peptides and other macromolecules. Biotinylation is rapid, specific and is unlikely to disturb the natural function of the molecule due to the small size of biotin (MW=244.31 g/mol). Proteins such as peptides can be biotinylated chemically for example by using amine-reactive biotinylation reagents, sulfhydryl biotinylation; or enzymatically, for example by fusion the protein of interest with an AviTag or acceptor peptide. Hence, a “biotinylated peptide” is a peptide that has been covalently attached to biotin.

The term “thiolyation” or “thiolated” as used herein refers to the process of attaching thiol groups, i.e. —SH functional groups to a protein, such as a peptide and other macromolecules. As the functional group of cysteine, the thiol group plays a very important role in biology. For example, when the thiol groups of two cysteine residues are brought together in the course of protein folding, an oxidation reaction can generate a cystine unit with a disulfide bond (—S—S—). Hence, a “thiolyated peptide” is a peptide which comprises thiol groups.

The term “thiol-metal interaction” used interchangeably with “metal-thiol interaction” as used herein refers to the metal-sulphur dative/co-ordinate bond formed between thiol-containing molecules and the metal ions found in a metal surface.

The term “Immunoglobulin” or “antibody” as used herein refers to a bivalent Y-shaped molecule comprising two identical heavy chains and two identical light chains. Disulfide bonds link together the heavy and light chain pairs as well as the two heavy chains. Each chain consists of one variable domain that varies in sequence and is responsible for antigen binding, these are known as the V_{H} and V_{L} domains for the heavy and light chains respectively. Each chain also consists of at least one constant domain. In the light chain there is a single constant domain (C_{L}) and in the heavy chain there are at least three (C_{H}1, C_{H}2 and C_{H}3), sometimes four (C_{H}4) depending on the isotype. In humans there are five different classes or isotypes of antibodies including IgA (which includes IgA1 and IgA2), IgD, IgE, IgG (which includes subclasses IgG1, IgG2, IgG3 and IgG4) and IgM.

The term “SARS-CoV-2 specific antibody” as used herein refers to an antibody, such as an IgG, which specifically binds to a SARS-CoV-2 target antigen, for example the S protein or N protein of SARS-CoV-2.

The term “identifying” as used herein in relation to an infection is to be interpreted broadly to encompass determining a presence, an absence, an amount, or a level of disease burden of the infection. The infection may be an infection in an acute phase, an early convalescent phase, a late convalescent phase, an early recovery phase, a late recovery phase or a full recovery phase.

The term “derived” as used herein in relation to a peptide is intended to indicate that the amino acid sequence of the peptide originated from the source specified, but has not necessarily been obtained directly from the specified source. For example, a “peptide derived from the S protein of SARS-CoV-2” refers to a peptide which has an amino acid sequence that is similar or identical to a part or a fragment of the S protein of SARS-CoV-2, or similar or identical to a part or a fragment of a consensus sequence of multiple naturally occurring SARS-CoV-2.

Thus, a “S protein-derived peptide” may be directly isolated from a naturally occurring S protein of SARS-CoV-2, or more typically it may be synthesized using the amino acid sequence of S protein of SARS-CoV-2 or a consensus sequence of multiple naturally occurring SARS-CoV-2 strains as a prototype/reference. The synthesis may be performed according to standard procedures in the art such as recombinant production techniques, genetic engineering techniques or chemical synthesis. A “S protein-derived peptide” can comprise artificial amino acids and non-natural amino acids (e.g. D-amino acids, amino acid analogues etc.). In some embodiments, a “S protein-derived peptide” may further comprise one or more conservative substitutions to the amino acid sequence originating from naturally occurring SARS-CoV-2 strains. The terms “N protein-derived peptide”, “S1 subunit-derived peptide”, or “S2 subunit-derived peptide” are to be construed similarly.

The term “subject” as used herein includes patients and non-patients. The term “patient” refers to individuals suffering or are likely to suffer from a medical condition such as a flavivirus infection, while “non-patients” refer to individuals not suffering and are likely to not suffer from the medical condition. “Non-patients” include healthy individuals, non-diseased individuals and/or an individual free from the medical condition. The term “subject” includes humans and animals. Animals include murine and the like. “Murine” refers to any mammal from the family Muridae, such as mouse, rat, and the like.

The term “treatment”, “treat” and “therapy”, and synonyms thereof as used herein refer to both therapeutic treatment and prophylactic or preventative measures, wherein the object is to prevent or slow down (lessen) a medical condition, which includes but is not limited to diseases (such as flavivirus infections), symptoms and disorders. A medical condition also includes a body's response to a disease or disorder, e.g. inflammation. Those in need of such treatment include those already with a medical condition as well as those prone to getting the medical condition or those in whom a medical condition is to be prevented.

The term “therapeutic agent‘ as used herein refers to a drug, protein, peptide, gene, chemical compound or other pharmaceutically active ingredient.

The term “and/or”, e.g., “X and/or Y” is understood to mean either “X and Y” or “X or Y” and should be taken to provide explicit support for both meanings or for either meaning.

Further, in the description herein, the word “substantially” whenever used is understood to include, but not restricted to, “entirely” or “completely” and the like. In addition, terms such as “comprising”, “comprise”, and the like whenever used, are intended to be non-restricting descriptive language in that they broadly include elements/components recited after such terms, in addition to other components not explicitly recited. For example, when “comprising” is used, reference to a “one” feature is also intended to be a reference to “at least one” of that feature. Terms such as “consisting”, “consist”, and the like, may in the appropriate context, be considered as a subset of terms such as “comprising”, “comprise”, and the like. Therefore, in embodiments disclosed herein using the terms such as “comprising”, “comprise”, and the like, it will be appreciated that these embodiments provide teaching for corresponding embodiments using terms such as “consisting”, “consist”, and the like. Further, terms such as “about”, “approximately” and the like whenever used, typically means a reasonable variation, for example a variation of +/−5% of the disclosed value, or a variance of 4% of the disclosed value, or a variance of 3% of the disclosed value, a variance of 2% of the disclosed value or a variance of 1% of the disclosed value.

Furthermore, in the description herein, certain values may be disclosed in a range. The values showing the end points of a range are intended to illustrate a preferred range. Whenever a range has been described, it is intended that the range covers and teaches all possible sub-ranges as well as individual numerical values within that range. That is, the end points of a range should not be interpreted as inflexible limitations. For example, a description of a range of 1% to 5% is intended to have specifically disclosed sub-ranges 1% to 2%, 1% to 3%, 1% to 4%, 2% to 3% etc., as well as individually, values within that range such as 1%, 2%, 3%, 4% and 5%. It is to be appreciated that the individual numerical values within the range also include integers, fractions and decimals. Furthermore, whenever a range has been described, it is also intended that the range covers and teaches values of up to 2 additional decimal places or significant figures (where appropriate) from the shown numerical end points. For example, a description of a range of 1% to 5% is intended to have specifically disclosed the ranges 1.00% to 5.00% and also 1.0% to 5.0% and all their intermediate values (such as 1.01%, 1.02% . . . 4.98%, 4.99%, 5.00% and 1.1%, 1.2% . . . 4.8%, 4.9%, 5.0% etc.,) spanning the ranges. The intention of the above specific disclosure is applicable to any depth/breadth of a range.

“At least 95% identical” as employed herein is intended to refer to an amino acid sequence which over its full length is 95% identical or more to a reference sequence, such as 96, 97, 98 or 99% identical. Software programmes can be employed to calculate percentage identity.

Additionally, when describing some embodiments, the disclosure may have disclosed a method and/or process as a particular sequence of steps. However, unless otherwise required, it will be appreciated that the method or process should not be limited to the particular sequence of steps disclosed. Other sequences of steps may be possible. The particular order of the steps disclosed herein should not be construed as undue limitations. Unless otherwise required, a method and/or process disclosed herein should not be limited to the steps being carried out in the order written. The sequence of steps may be varied and still remain within the scope of the disclosure.

Furthermore, it will be appreciated that while the present disclosure provides embodiments having one or more of the features/characteristics discussed herein, one or more of these features/characteristics may also be disclaimed in other alternative embodiments and the present disclosure provides support for such disclaimers and these associated alternative embodiments.

### DESCRIPTION OF EMBODIMENTS

It will be appreciated by a person skilled in the art that other variations and/or modifications may be made to the embodiments disclosed herein without departing from the spirit or scope of the disclosure as broadly described. For example, in the description herein, features of different exemplary embodiments may be mixed, combined, interchanged, incorporated, adopted, modified, included etc. or the like across different exemplary embodiments. The present embodiments are, therefore, to be considered in all respects to be illustrative and not restrictive.

In one aspect, there is provided a nanosensor for detecting SARS-CoV-2 antibodies, comprising a metal nanoparticle functionalized with one or more B-cell linear peptide epitopes derived from the spike (S) protein or nucleocapsid (N) protein of SARS-CoV-2.

Accordingly, the present inventors have developed a nanoparticle (NP) based nanosensor for detecting SARS-CoV-2-specific antibodies using SARS-CoV-2 B-cell linear peptide epitopes. Advantageously, this hybrid nanobiosensor concept exploits both the distance-dependent optical properties of NPs and the natural IgG recognition ability of immunodominant epitopes derived from the S and/or N proteins of SARS-CoV-2 to enable a rapid serological assay, without the use of recombinant viral proteins or antihuman antibodies.

This is an important advantage because the use of recombinant full-length viral proteins may result in ambiguous detection outcome due to cross-reactivity with phylogenetically similar viruses. (17) In particular, both the S and N proteins share a high homology with other human coronaviruses (hCoVs); for instance, they show 76% and 90% sequence similarity with SARS-CoV S and N proteins, respectively. (15) This sequence homology contributes to significant cross-reactivity in serological tests employing recombinant viral proteins. (16) Full-length recombinant antigens may also suffer from batch-to-batch variations and stability problems during storage and transport, affecting the reproducibility of serological immunoassays. (17) In addition, the use of small peptides is also simpler and cheaper compared to the production of entire S or N proteins, as it can be achieved via high-throughput chemical methods such as solid-state synthesis without requiring the use of genetically modified organisms or cell culture. Further advantageously, due to the smaller size of peptide epitopes (˜20 amino acids), a single nanoparticle can be functionalized with multiple peptide epitopes. As such, the presently disclosed nanosensors have the ability to detect a pool of antibodies against SARS-CoV-2 S and N proteins, including subunits (S1 and S2), which is not possible with other competing protein-based immunoassays.

In some embodiments, the one or more epitopes are conjugated to the nanoparticle surface via a biotin-streptavidin interaction, via a thiol-metal interaction wherein the nanoparticle is coated with a spacer comprising at least 4 amino acids and at least one cysteine residue, or via EDC/NHS coupling of amine-terminated peptides to nanoparticles with a COOH group (for example, MUA, 11-Mercaptoundecanoic acid functionalized nanoparticles).

In one embodiment, the one or more epitopes are conjugated to the nanoparticle surface via:
a) a biotin-streptavidin interaction, or
b) via a thiol-metal interaction wherein the nanoparticle is coated with a spacer comprising at least 4 amino acids and at least one cysteine residue.

Advantageously, peptide conjugation to nanoparticles, such as gold nanoparticles (AuNP), either via a streptavidin-biotin or thiol-metal interaction presents a more direct and straightforward approach to control the orientation and retain the bioactivity relative to protein- and antibody-based assays. Thus, in this respect, the present disclosure provides a reliable means of functionalizing metal nanoparticles with SARS-CoV-2 B-cell linear peptide epitopes.

In one embodiment, the one or more peptide epitopes are conjugated via biotin-streptavidin interaction. In one embodiment, the one or more peptide epitopes are conjugated via biotin-streptavidin interaction, wherein the metal nanoparticle is coated with streptavidin and the epitopes are biotinylated. Advantageously, the biotin-streptavidin interaction results in a very strong and stable bond between the linear peptide epitopes and the nanoparticle, which is resistant to extremes of heat, pH and proteolysis. This makes the nanosensors of the present disclosure particularly suitable for use in the assays and methods of the present disclosure.

In one embodiment, the one or more peptide epitopes are conjugated via a thiol-metal interaction. In one embodiment, the one or more peptide epitopes are conjugated via a thiol-gold interaction.

In one embodiment, the one or more peptide epitopes are conjugated via a thiol-metal interaction, wherein the peptide epitopes are thiolyated. In one embodiment, the one or more peptide epitopes are conjugated via a thiol-metal interaction, wherein the peptide epitopes are thiolyated and the metal nanoparticle is coated with a thiolyated spacer, such as a short peptide spacer.

In one embodiment, the short peptide spacer comprises at least 4 amino acids, such as 5, 6, 7, 8, 9 or 10 amino acids. In one embodiment, the short peptide spacer comprises 5 amino acids. In one embodiment, the spacer comprises at least one terminal cysteine residue. In one embodiment, the spacer comprises at least 4 amino acids and at least one cysteine residue. In one embodiment, the spacer comprises at least 4 amino acids and at least one terminal cysteine residue.

In one embodiment, the short peptide spacer comprises an amino acid sequence CALNN. Advantageously, the present inventors have established that the presence of the spacer helps ensure that the peptide epitopes are properly spaced apart from each other on the nanoparticle surface, thereby allowing the nanosensor to effectively bind to the target SARS-CoV-2 antibodies.

Thus, in one embodiment, the one or more peptide epitopes are conjugated via a thiol-metal interaction, wherein the peptide epitopes are thiolyated and the metal nanoparticle is coated with a thiolyated spacer comprising the amino acid sequence CALNN.

In one embodiment, the nanosensor is a bio nanosensor.

In one embodiment, the antibody is selected from the group comprising of IgA, IgD, IgE, IgG and IgM. In one embodiment, the antibody is an IgG molecule. Thus, in one embodiment the SARS-CoV-2 specific antibody is a SARS-CoV-2 specific IgG.

In one embodiment, the SARS-CoV-2 specific antibody is an anti-S protein antibody, such as an anti-S protein IgG. In one embodiment, the SARS-CoV-2 specific antibody is an anti-S1 subunit protein, such as an anti-S1 subunit IgG. In one embodiment, the SARS-CoV-2 specific antibody is an anti-S2 subunit protein, such as an anti-S2 subunit IgG. In one embodiment, the SARS-CoV-2 specific antibody is an anti-N protein antibody, such as an anti-N protein IgG.

In one embodiment, the metal nanoparticle is selected from the group consisting of gold, silver, platinum, titanium, zinc, cerium, iron, thallium, and a combination thereof.

In one embodiment, the metal nanoparticle is selected from the group consisting of gold, silver, and a combination of gold and silver, such as Au₅₀Ag₅₀. In one embodiment, the metal is selected from the group consisting of gold, silver, and a combination of gold and silver, such as Au₅₀Ag₅₀. Advantageously, the present inventors have demonstrated that metal nanoparticles comprising these metals are particularly suitable for use in the disclosed nanosensors.

In one embodiment, the metal nanoparticle is a gold nanoparticle (AuNP). The advantage of employing gold nanoparticles is that gold nanoparticles are one of the most stable, non-toxic, easy to synthesize, and well-characterised nanoparticles, which have been widely used in biomedical applications.

In one embodiment, the metal nanoparticle is a silver nanoparticle (AgNP).

In one embodiment, the metal nanoparticle is a platinum nanoparticle. In one embodiment, the metal nanoparticle is a titanium nanoparticle. In one embodiment, the metal nanoparticle is a zinc nanoparticle. In one embodiment, the metal nanoparticle is a cerium nanoparticle. In one embodiment, the metal nanoparticle is an iron nanoparticle.

In one embodiment, the metal nanoparticle is a thallium nanoparticle.

In one embodiment, the metal nanoparticle is combination of pure metals, for example a combination of gold and silver, a combination of gold and platinum, a combination of gold and titanium, a combination of gold and zinc, a combination of gold and cerium, a combination of gold an iron, a combination of gold and thallium.

In one embodiment, the metal nanoparticle is a gold and silver nanoparticle (AuAg), such as Au₁₀Ag₉₀, Au₂₀Ag₈₀, Au₃₀Ag₇₀, Au₄₀Ag₆₀, Au₅₀Ag₅₀, Au60Ag₄₀, Au₇₀Ag₃₀, Au₈₀Ag₂₀, Au₉₀Ag₁₀.

In one embodiment, the metal nanoparticle is a Au₅₀Ag₅₀ nanoparticle.

In one embodiment, the metal nanoparticle is spherical, substantially spherical, or non-spherical, such as irregularly shaped particles or ellipsoidally shaped particle.

In one embodiment, the metal nanoparticle is spherical.

In one embodiment, the metal nanoparticle has a diameter of 5 to 20 nm, such as 5 to 20 nm, 5 to 19 nm, 5 to 18 nm, 5 to 17 nm, 5 to 16 nm, 5 to 15 nm, 5 to 14 nm, 5 to 13 nm, 5 to 12 nm, 5 to 11 nm, 5 to 10 nm, 5 to 9 nm, 5 to 8 nm, 5 to 7 nm, or 5 to 6 nm.

In one embodiment, the metal nanoparticle has a diameter of 13 nm.

In one embodiment, the metal nanoparticle has a diameter of 5 nm. In one embodiment, the metal nanoparticle has a diameter of 6 nm. In one embodiment, the metal nanoparticle has a diameter of 7 nm. In one embodiment, the metal nanoparticle has a diameter of 7 nm. In one embodiment, the metal nanoparticle has a diameter of 8 nm. In one embodiment, the metal nanoparticle has a diameter of 7 nm. In one embodiment, the metal nanoparticle has a diameter of 9 nm. In one embodiment, the metal nanoparticle has a diameter of 10 nm. In one embodiment, the metal nanoparticle has a diameter of 11 nm. In one embodiment, the metal nanoparticle has a diameter of 12 nm. In one embodiment, the metal nanoparticle has a diameter of 14 nm. In one embodiment, the metal nanoparticle has a diameter of 15 nm. In one embodiment, the metal nanoparticle has a diameter of 16 nm. In one embodiment, the metal nanoparticle has a diameter of 17 nm. In one embodiment, the metal nanoparticle has a diameter of 18 nm. In one embodiment, the metal nanoparticle has a diameter of 19 nm. In one embodiment, the metal nanoparticle has a diameter of 20 nm.

In one embodiment, the metal nanoparticle is functionalized with 1 to 30 linear peptide epitopes, such as 1 to 29, 1 to 28, 1 to 27, 1 to 26, 1 to 25, 1 to 24, 1 to 23, 1 to 22, 1 to 21, 1 to 20, 1 to 19, 1 to 18, 1 to 17, 1 to 16, 1 to 15, 1 to 14, 1 to 13, 1 to 12, 1 to 11, 1 to 10, 1 to 9, 1 to 8, 1 to 7, 1 to 8, 1 to 6, 1 to 5, 1 to 4, 1 to 3, or 1 to 2 linear peptide epitopes.

In one embodiment, the metal nanoparticle is functionalized with up to 20 linear peptide epitopes.

In one embodiment, the metal nanoparticle is functionalized with 1 linear peptide epitope.

In one embodiment, the metal nanoparticle is functionalized with 2 linear peptide epitopes.

In one embodiment, the metal nanoparticle is functionalized with 3 linear peptide epitopes. In one embodiment, the metal nanoparticle is functionalized with 4 linear peptide epitopes. In one embodiment, the metal nanoparticle is functionalized with 5 linear peptide epitopes. In one embodiment, the metal nanoparticle is functionalized with 6 linear peptide epitopes. In one embodiment, the metal nanoparticle is functionalized with 7 linear peptide epitopes. In one embodiment, the metal nanoparticle is functionalized with 8 linear peptide epitopes. In one embodiment, the metal nanoparticle is functionalized with 9 linear peptide epitopes. In one embodiment, the metal nanoparticle is functionalized with 10 linear peptide epitopes. In one embodiment, the metal nanoparticle is functionalized with 11 linear peptide epitopes. In one embodiment, the metal nanoparticle is functionalized with 12 linear peptide epitopes. In one embodiment, the metal nanoparticle is functionalized with 13 linear peptide epitopes. In one embodiment, the metal nanoparticle is functionalized with 14 linear peptide epitopes. In one embodiment, the metal nanoparticle is functionalized with 15 linear peptide epitopes. In one embodiment, the metal nanoparticle is functionalized with 16 linear peptide epitopes. In one embodiment, the metal nanoparticle is functionalized with 17 linear peptide epitopes. In one embodiment, the metal nanoparticle is functionalized with 18 linear peptide epitopes. In one embodiment, the metal nanoparticle is functionalized with 19 linear peptide epitopes. In one embodiment, the metal nanoparticle is functionalized with 20 linear peptide epitopes.

In various embodiments, the length/size of the peptide is no more than about 30, no more than about 29, no more than about 28, no more than about 27, no more than about 26, no more than about 25, no more than about 24, no more than about 23, no more than about 22, no more than about 21, no more than about 20, no more than about 19, no more than about 18, no more than about 17, no more than about 16, no more than about 15, no more than about 14, no more than about 13, no more than about 12, no more than about 11, no more than about 10, no more than about 9, no more than about 8, no more than about 7, no more than about 6 or no more than about 5 amino acids long. various embodiments, the length/size of the peptide is from about 5 to about 30, from about 10 to about 25 or from about 15 to about 20 amino acids long. In one embodiment, the peptide is from about 5 to about 25 amino acids long. In some embodiments, the length/size of the peptide is about 10, about 11, about 12, about 13, about 14, about 15, about 16, about 17, about 18, about 19, about 20 or about 21 amino acids long.

In one embodiment, the epitope is derived from the spike protein of SARS-CoV-2. In one embodiment, the epitope is derived from the amino acid sequence given in SEQ ID NO: 1. The advantage of deriving the epitope from the spike protein is that the N protein of SARS-CoV-2 shares ˜90% homology in amino acid sequences with that of SARS-CoV. Thus, the likelihood of cross-reactivity with SARs-CoV is reduced by deriving the epitope from the S protein vs the N protein. In other words, functionalizing the nanoparticle with S protein epitopes is more likely to increase the specificity of the nanosensor for SARS-CoV-2 antibodies.

In one embodiment, the epitope is derived from the nucleocapsid protein of SARS-CoV-2. In one embodiment, the epitope is derived from the amino acid sequence given in SEQ ID NO: 2.

In one embodiment, the epitope is derived from the S1 or S2 subunit of the spike protein. Thus, in one embodiment, the epitope is derived from the S1 subunit of the spike protein. Thus, in one embodiment, the epitope is derived from the S2 subunit of the spike protein.

In one embodiment, the epitope comprises an amino acid sequence selected from the group consisting of:

(S14P5)
(SEQ ID NO: 3)
TESNKKFLPFQQFGRDIA,
(S20P2)
(SEQ ID NO: 4)
GIAVEQDKNTQEVFAQVK,
(S21P2)
(SEQ ID NO: 5)
PSKPSKRSFIEDLLFNKV,
(N4P5)
(SEQ ID NO: 6)
NNAAIVLQLPQGTTLPKG,

and
an amino acid sequence at least 95% identical to one of the above.

Advantageously, these 4 linear B-cell epitopes are highly conserved within the presently circulating COVID-19 variants and have no detectable cross-reactivity against seasonal hCoV and recovered SARS-CoV patients. Thus, these 4 linear epitopes are particularly useful for functionalizing the metal nanoparticles of the presently disclosed nanosensor.

In one embodiment, the epitope comprises an amino acid sequence selected from the group consisting of:

(S14P5)
(SEQ ID NO: 3)
TESNKKFLPFQQFGRDIA,
(S20P2)
(SEQ ID NO: 4)
GIAVEQDKNTQEVFAQVK,
(S21P2)
(SEQ ID NO: 5)
PSKPSKRSFIEDLLFNKV,

and
an amino acid sequence at least 95% identical to one of the above.

In one embodiment, the epitope comprises an amino acid sequence selected from the group consisting of:

(S14P5)
(SEQ ID NO: 3)
TESNKKFLPFQQFGRDIA,
(S20P2)
(SEQ ID NO: 4)
GIAVEQDKNTQEVFAQVK,

and
an amino acid sequence at least 95% identical to one of the above.

Advantageously, the present inventors have established that these 2 epitopes have a particularly high binding affinity (Kd in pM range) for SARS-CoV-2 antibodies.
In one embodiment, the epitope comprises an amino acid sequence selected from the group consisting of:

(S14P5)
(SEQ ID NO: 3)
TESNKKFLPFQQFGRDIA,
(S21P2)
(SEQ ID NO: 5)
PSKPSKRSFIEDLLFNKV,

and
an amino acid sequence at least 95% identical to one of the above.

In one embodiment, the epitope comprises an amino acid sequence selected from the group consisting of:

(S14P5)
(SEQ ID NO: 3)
TESNKKFLPFQQFGRDIA,
(S20P2)
(SEQ ID NO: 4)
GIAVEQDKNTQEVFAQVK,
(S21P2)
(SEQ ID NO: 5)
PSKPSKRSFIEDLLFNKV,
(N4P5)
(SEQ ID NO: 6)
NNAAIVLQLPQGTTLPKG.

In one embodiment, the epitope comprises an amino acid sequence TESNKKFLPFQQFGRDIA (S14P5) (SEQ ID NO: 3) or an amino acid sequence at least 95% identical thereto. In one embodiment, the epitope comprises an amino acid sequence TESNKKFLPFQQFGRDIA (S14P5) (SEQ ID NO: 3). Advantageously, the present inventors have determined that nanosensors functionalized with this particular epitope have the lowest limit of detection (LOD of 4.1 nM, vs 5.1 nM for S21P2, 15.4 nM for S20P2 and 25.8 nM for N45P5) of the 4 peptide epitopes.

In one embodiment, the epitope comprises an amino acid sequence GIAVEQDKNTQEVFAQVK (S20P2) (SEQ ID NO: 4) or an amino acid sequence at least 95% identical thereto. In one embodiment, the epitope comprises an amino acid sequence GIAVEQDKNTQEVFAQVK (S20P2) (SEQ ID NO: 4).

In one embodiment, the epitope comprises an amino acid sequence PSKPSKRSFIEDLLFNKV (S21P2) (SEQ ID NO: 5) or an amino acid sequence at least 95% identical thereto. In one embodiment, the epitope comprises an amino acid sequence PSKPSKRSFIEDLLFNKV (S21P2) (SEQ ID NO: 5).

In one embodiment, the epitope comprises an amino acid sequence NNAAIVLQLPQGTTLPKG (N4P5) (SEQ ID NO: 6) or an amino acid sequence at least 95% identical thereto. In one embodiment, the epitope comprises an amino acid sequence NNAAIVLQLPQGTTLPKG (N4P5) (SEQ ID NO: 6).

In one embodiment, the metal nanoparticle is functionalized with at least 2 different linear peptide epitopes, such as at least 2 to 30 different epitopes, for example 2 to 29, 2 to 28, 2 to 27, 2 to 26, 2 to 25, 2 to 24, 2 to 23, 2 to 22, 2 to 21, 2 to 20, 2 to 19, 2 to 18, 2 to 17, 2 to 16, 2 to 15, 2 to 14, 2 to 13, 2 to 12, 2 to 11, 2 to 10, 2 to 9, 2 to 8, 2 to 7, 2 to 6, 2 to 5, 2 to 4, or 2 to 3 different epitopes. The major benefit of functionalizing the nanoparticle with multiple epitopes is that this enables the nanosensor to detect a wider range of different SARS-CoV-2 antibodies, thereby significantly enhancing the sensitivity of the nanosensor.

In one embodiment, the metal nanoparticle is functionalized with 3 different linear peptide epitopes. In one embodiment, the metal nanoparticle is functionalized with 4 different linear peptide epitopes. In one embodiment, the metal nanoparticle is functionalized with 5 different linear peptide epitopes. In one embodiment, the metal nanoparticle is functionalized with 6 different linear peptide epitopes. In one embodiment, the metal nanoparticle is functionalized with 7 different linear peptide epitopes. In one embodiment, the metal nanoparticle is functionalized with 8 different linear peptide epitopes. In one embodiment, the metal nanoparticle is functionalized with 9 different linear peptide epitopes. In one embodiment, the metal nanoparticle is functionalized with 10 different linear peptide epitopes. In one embodiment, the metal nanoparticle is functionalized with 11 different linear peptide epitopes. In one embodiment, the metal nanoparticle is functionalized with 12 different linear peptide epitopes. In one embodiment, the metal nanoparticle is functionalized with 13 different linear peptide epitopes. In one embodiment, the metal nanoparticle is functionalized with 14 different linear peptide epitopes. In one embodiment, the metal nanoparticle is functionalized with 15 different linear peptide epitopes. In one embodiment, the metal nanoparticle is functionalized with 16 different linear peptide epitopes. In one embodiment, the metal nanoparticle is functionalized with 17 different linear peptide epitopes. In one embodiment, the metal nanoparticle is functionalized with 18 different linear peptide epitopes. In one embodiment, the metal nanoparticle is functionalized with 19 different linear peptide epitopes. In one embodiment, the metal nanoparticle is functionalized with 20 different linear peptide epitopes.

In one embodiment, the metal nanoparticle is functionalized with 2 different linear peptide epitopes.

In one embodiment, at least one epitope is derived from the S1 subunit of the spike protein, and at least one other epitope is derived from the S2 subunit of the spike protein. The advantage of functionalizing the nanoparticles with epitopes from both subunits is that provides the nanosensor with the ability to detect both anti-S1 subunit and anti-S2 subunit SARS-CoV-2 antibodies. This helps to improve the sensitivity of the nanosensor.

In one embodiment, the 2 different linear peptide epitopes comprise an amino acid sequence selected from the group consisting of:

(S14P5)
(SEQ ID NO: 3)
TESNKKFLPFQQFGRDIA,
(S20P2)
(SEQ ID NO: 4)
GIAVEQDKNTQEVFAQVK,
(S21P2)
(SEQ ID NO: 5)
PSKPSKRSFIEDLLFNKV,
(N4P5)
(SEQ ID NO: 6)
NNAAIVLQLPQGTTLPKG,

and
an amino acid sequence at least 95% identical to one of the above.

In one embodiment, the two different epitopes are TESNKKFLPFQQFGRDIA (S14P5) (SEQ ID NO: 3) or an amino acid sequence at least 95% identical thereto, and GIAVEQDKNTQEVFAQVK (S20P2) (SEQ ID NO: 4) or an amino acid sequence at least 95% identical thereto.

In one embodiment, the two different epitopes are TESNKKFLPFQQFGRDIA (S14P5) (SEQ ID NO: 3) or an amino acid sequence at least 95% identical thereto, and PSKPSKRSFIEDLLFNKV (S21P2) (SEQ ID NO: 5) or an amino acid sequence at least 95% identical thereto.

Advantageously, the present inventors have demonstrated that nanosensors featuring nanoparticles that have been functionalized with the above 2 pairs of peptide epitopes have increased nanosensor response.

In one embodiment, the two different epitopes are TESNKKFLPFQQFGRDIA (S14P5) (SEQ ID NO: 3) or an amino acid sequence at least 95% identical thereto, and NNAAIVLQLPQGTTLPKG (N4P5) (SEQ ID NO: 6) or an amino acid sequence at least 95% identical thereto.

In one embodiment, the two different epitopes are GIAVEQDKNTQEVFAQVK (S20P2) (SEQ ID NO: 4) or an amino acid sequence at least 95% identical thereto, and PSKPSKRSFIEDLLFNKV (S21P2) (SEQ ID NO: 5) or an amino acid sequence at least 95% identical thereto.

In one embodiment, the two different epitopes are GIAVEQDKNTQEVFAQVK (S20P2) (SEQ ID NO: 4) or an amino acid sequence at least 95% identical thereto, and NNAAIVLQLPQGTTLPKG (N4P5) (SEQ ID NO: 6) or an amino acid sequence at least 95% identical thereto.

In one embodiment, the two different epitopes are GIAVEQDKNTQEVFAQVK (S20P2) (SEQ ID NO: 4) or an amino acid sequence at least 95% identical thereto, and PSKPSKRSFIEDLLFNKV (S21P2) (SEQ ID NO: 5) or an amino acid sequence at least 95% identical thereto.

In one embodiment, the two different epitopes are TESNKKFLPFQQFGRDIA (S14P5) (SEQ ID NO: 3), and GIAVEQDKNTQEVFAQVK (S20P2) (SEQ ID NO: 4).

In one embodiment, the two different epitopes are TESNKKFLPFQQFGRDIA (S14P5) (SEQ ID NO: 3), and PSKPSKRSFIEDLLFNKV (S21P2) (SEQ ID NO: 5).

In one embodiment, the two different epitopes are TESNKKFLPFQQFGRDIA (S14P5) (SEQ ID NO: 3), and NNAAIVLQLPQGTTLPKG (N4P5) (SEQ ID NO: 6).

In one embodiment, the two different epitopes are GIAVEQDKNTQEVFAQVK (S20P2) (SEQ ID NO: 4), and PSKPSKRSFIEDLLFNKV (S21P2) (SEQ ID NO: 5).

In one embodiment, the two different epitopes are GIAVEQDKNTQEVFAQVK (S20P2) (SEQ ID NO: 4), and NNAAIVLQLPQGTTLPKG (N4P5) (SEQ ID NO: 6).

In one embodiment, the two different epitopes are GIAVEQDKNTQEVFAQVK (S20P2) (SEQ ID NO: 4), and PSKPSKRSFIEDLLFNKV (S21P2) (SEQ ID NO: 5).

In one embodiment, the nanoparticle is passivated.

In one embodiment, the nanoparticle is passivated with a substance suitable for passivating the nanoparticle against biofluids, for example blood, saliva, mucus, plasma or serum.

In one embodiment, the nanoparticle is passivated through electrostatic repulsion, for example by coating the nanoparticles with citrate.

In one embodiment, the nanoparticle is citrate-coated.

In one embodiment, the nanoparticle is passivated by coating the nanoparticle with a polymer layer, such as polyethylene glycol (PEG), polyvinyl pyrrolidone (PVP).

In one embodiment, the nanoparticle is passivated by coating the nanoparticle with a polyethylene glycol (PEG) layer. Surprisingly, the present inventors have shown that passivating the nanoparticles with PEG moieties significantly improved nanosensor response in high plasma background samples.

In one embodiment, the nanoparticle is passivated by coating the nanoparticle with a zwitterionic ligand, such as a carboxybetaine or a sulfobetaine. Thus, in one embodiment, the nanoparticle is coated with polycarboxybetaine.

In one embodiment, the nanoparticle is passivated by coating the nanoparticle with a lipid bilayer, such as a lipid bilayer of 1,2-dimyristoyl-sn-glycero-3-phosphocholine (DMPC). Thus, in one embodiment, the nanoparticle is coated with DMPC.

In one embodiment, the nanoparticle is passivated by coating the nanoparticle with a sugar layer, such as a glycan layer, for example with thiol-terminated glycoconjugates such as N-acetylglucosamine (GlcNAc) or disaccharide lactose (Lac).

Thus, in one embodiment, the nanoparticle is coated with a thiol-terminated glycoconjugate. In some other embodiments, the nanoparticle is coated with a protein or amino acid layer, such as glycine, or short peptides.

In one aspect, there is provided an in vitro assay for detecting the presence of SARS-CoV-2 specific antibodies in a sample, comprising a plurality of nanosensors as defined above.

In one embodiment, the assay further comprises one or more of the following: a stabilizer (such as Tween 20), a buffer (such as PBS), a control antibody (such as a SARS-CoV-2 antibody, a SARS-CoV antibody or a normal human IgG), a microplate, and combinations thereof. Thus, in one embodiment, the assay further comprises a stabilizer, a buffer, a control antibody and a microplate.

In one embodiment, the stabilizer is Tween, such as Tween 20, Tween 40 or Tween 80. In one embodiment, the stabilizer is Tween 40. In one embodiment, the stabilizer is Tween 80. In one embodiment, the stabilizer is 0.01-10% Tween. In one embodiment, the stabilizer is 0.05% Tween.

In one embodiment, the stabilizer is Tween 20, such as 0.01-10% Tween 20. In one embodiment, the stabilizer is 0.01-10% Tween 20. In one embodiment, the stabilizer is 0.05% Tween 20.

In one embodiment, the assay comprises wherein the control antibody is selected from the group consisting of a SARS-CoV-2 IgG antibody (such as a COVID-19 anti-S1 or anti-S2 antibody), a SARS-CoV IgG antibody (such as a SARS anti-S1 antibody) and a normal human IgG. In one embodiment, the control antibody is a SARS-CoV-2 antibody. In one embodiment, the control antibody is a COVID-19 anti-S1 or anti-S2 antibody. In one embodiment, the control antibody is a COVID-19 anti-S1 antibody. In one embodiment, the control antibody is a COVID-10 anti-S2 antibody. In one embodiment, the control antibody is a normal human antibody, such as a normal human IgG. In one embodiment, the assay comprises more than one control antibody, for example one or more of the abovementioned control antibodies.

In one embodiment, the buffer is selected from the group consisting of TRIS, HEPES, PIPES, MOPS, MES, and PBS. In one embodiment, the buffer is TRIS. In one embodiment, the buffer is HEPES. In one embodiment, the buffer is PIPES. In one embodiment, the buffer is MOPS. In one embodiment, the buffer is MES.

In one embodiment, the buffer is PBS. In one embodiment the buffer is 1×PBS.

In one embodiment, the microplate is a 96-well microplate or a 384-well microplate. In one embodiment, the microplate is a 96-well microplate. In one embodiment, the microplate is a 384-well microplate

In one aspect, there is provided an in vitro method of detecting the presence of SARS-CoV-2 specific antibodies in a sample, comprising contacting the sample with a plurality of nanosensors as defined above, and detecting the degree of nanoparticle aggregation.

In one aspect, there is provided an in vitro method of diagnosing a subject as having a SARS-CoV-2 infection, comprising:
detecting the presence of SARS-CoV-2 specific antibodies in a sample from the subject by contacting the sample with a plurality of nanosensors as defined above,
detecting the degree of nanoparticle aggregation in the sample,
comparing the degree of nanoparticle aggregation in the sample to the degree of nanoparticle aggregation in a control sample from a subject that does not have a SARS-CoV-2 infection,
determining if the degree of nanoparticle aggregation is higher in the sample compared to the degree of nanoparticle aggregation in the control sample, thereby diagnosing the subject as having a SARS-CoV-2 infection.

The presently disclosed assay and methods have numerous benefits over existing prior art assays/serological tests, including but not limited to:
1. The assay/method is able to detect SARS-CoV-2 in a sample with a high degree of sensitivity/specificity;
2. The assay/method only requires a single reagent, i.e. the disclosed nanosensors. Hence, this helps to reduce reduces manufacturing time and costs, reduces packaging, transport costs, etc.
3. The assay/method requires very small amounts of sample (as low as 10 μl);
4. The assay/method is simple to perform since it only requires a simple mixing step and hence can be performed by untrained personnel;
5. The assay/method results in visible nanoparticle aggregation, which is very obvious and can be observed with the naked eye;
6. The assay/method can be used with standard 96 or 384 well microplates and therefore a quantitative readout can be obtained using a standard microplate reader which is readily available in basic labs or hospital settings.

These various technical advantages therefore make the presently disclosed assay/method particularly suitable for front-line COVID-19 testing, for example at ports, airports, hospitals, etc, as well as in resource poor environments such as rural communities or less developed countries.

In one aspect, there is provided a method of treating a subject identified as having a SARS-CoV-2 infection, comprising:
detecting the presence of SARS-CoV-2 specific antibodies in a sample from the subject by contacting the sample with a plurality of nanosensors as defined above,
detecting the degree of nanoparticle aggregation in the sample,
comparing the degree of nanoparticle aggregation in the sample to the degree of nanoparticle aggregation in a control sample from a subject that does not have a SARS-CoV-2 infection,
determining if the degree of nanoparticle aggregation is higher in the sample compared to the degree of nanoparticle aggregation in the control sample, thereby identifying the subject as having a SARS-CoV-2 infection; and
administering a therapeutic agent, such as an anti-viral agent to the subject.

Advantageously, the presently disclosed method of treatment allows a practitioner to have a high degree of confidence that a subject is a true positive COVID-19 patient. This therefore reduces the probability of administering an unnecessary COVID-19 treatment to a subject who is actually COVID-19 negative; and also reduces the likelihood of failing to administer an appropriate COVID-19 treatment to a COVID-19 positive patient who requires such treatment.

In one embodiment, the sample is a biofluid, fluid biological sample or liquid biological sample. The biofluid, sample may be blood, serum, plasma, sputum, lavage fluid, cerebrospinal fluid, urine, semen, sweat, tears, saliva, mucus and the like. In some embodiments, the biofluid comprises whole blood, blood serum, blood plasma or processed fractions thereof.

In one embodiment, the sample is saliva.

In one embodiment, the sample is mucus, for example obtained from a nasal swab.

In one embodiment, the fluid biological sample comprises blood serum or blood plasma.

In one embodiment, the sample is plasma. Advantageously, the present assay/method can be used to detect SARS-CoV-2 antibodies in samples with high plasma background, with minimal impact on sensor responsiveness.

In various embodiments, the sample is collected from a subject during an acute phase (about 2 days to about 7 days post illness onset (pio)), an early convalescent phase (about 10 days to about 14 days pio), a late convalescent phase (about 1 month pio), an early recovery phase (about 3 months pio), a late recovery phase (about 5 months to about 6 months pio) or a full recovery phase (about 1 year pio).

In one embodiment, the sample is collected from a subject who exhibits symptoms of a SARS-CoV-2 infection, for one or more of the following symptoms: fever, cough, tiredness, loss of taste and/or smell, sore throat, headaches, aches and pains, diarrhoea, a rash on skin or discolouration of fingers/toes, red or irritated eyes, and chest pain.

In one embodiment, the sample is collected from a subject who is or has become asymptomatic for a SARS-CoV-2 infection.

In one embodiment, the degree of nanoparticle aggregation is proportional to the quantity of SARs-CoV-2 specific antibodies, such as SARs-CoV-2 specific IgGs. In one embodiment, the degree of nanoparticle aggregation is proportional to the quantity of anti-S protein antibodies and/or anti-N protein antibodies in the sample. In one embodiment, the degree of nanoparticle aggregation is proportional to the quantity of anti-S1 subunit antibodies or anti-S2 subunit antibodies in the sample. In one embodiment, the degree of nanoparticle aggregation is proportional to the quantity of anti-N protein antibodies in the sample. This is a major benefit of the presently disclosed assay/method, which allows accurate quantification of SARS-CoV-2 specific antibodies in the sample. This for example allows for an assessment of the severity of disease in a subject, which can help to better guide the medical practitioner as to the proper intervention to take.

In one embodiment, the limit of detection is about 3 nM, such as about 3.2 nM. Thus, in one embodiment, the minimum quantity of SARS-CoV-2 specific antibodies that can be detected in a sample is about 3 nM, such as 3.2 nM.

In one embodiment, the range of SARS-CoV-2 specific antibodies that can be detected in a sample is about 3-1000 nM, such as 3.2-1000 nM, such as 10-1000 nM, such as 50-1000 nM, 100-1000 nM, 200-1000 nM, 300-1000 nM, 400-1000 nM, 500-1000 nM, 600-1000 nM, 700-1000 nM, 800-1000 nM, or 900-1000 nM. In one embodiment, the quantity of SARS-CoV-2 specific antibodies in the sample is less than 100 nM, such as 3, 4, 5, 6, 7, 8, 9, 10, 20, 30, 40, 50, 60, 70, 80, 90 or 100 nM. In one embodiment, the sample comprises more than 100 nM SARS-CoV-2 specific antibodies, for example, 100, 150, 200, 250, 300, 350, 400, 450, 500, 550, 600, 650, 700, 750, 800, 850, 900, 950 or 1000 nM SARS-CoV-2 specific antibodies. The SARS-CoV-2 concentration typically reaches a peak during 21-25 days after illness onset as 16.5 μg/mL and stays at a relatively high concentration (11.4 μg/mL) until 41 days. Thus, the presently disclosed assay/method is suitable for early infection detection and for onset status determination, according to the IgG profile in a typical COVID-19 patient.

In one embodiment, the nanoparticle aggregation results in a visible colour change, for example a colour change that is visible to the naked eye. In one embodiment, the nanoparticle aggregation results in a visible colour, if the quantity of SARS-CoV-2 specific antibodies in the sample is above a certain threshold. In one embodiment, the threshold is about 10 to 25 nM, such as 10 nM, 15 nM, 20 nM, or 25 nM. In one embodiment, the threshold is about 10 nM. Thus, in one embodiment, the nanoparticle aggregation results in a visible colour change if the quantity of SARS-CoV-2 specific antibodies in the sample is above 10 nM.

Advantageously, the presently disclosed assay/method results in an obvious visual change which is often visible with the naked eye. This eliminates the requirement for additional equipment in order to obtain a readout on the result of the assay/method.

In one embodiment, the nanoparticle aggregation results in a colour change from red to purple. In one embodiment, the nanoparticle aggregation results in a red to purple colour change if the quantity of SARS-CoV-2 specific antibodies in the sample is above the threshold for a visible colour change. Thus, in one embodiment, the nanoparticle aggregation results in a red to purple colour change if the quantity of SARS-CoV-2 specific antibodies in the sample is above 10 nM, such as above 25 nM, above 50 nM, above 75 nM, or above 100 nM.

In one embodiment, the nanoparticle aggregation colour change is not visible to the naked eye.

In one embodiment, the degree of nanoparticle aggregation is assessed by determining the % change in A_{534 nm}, for example by detecting the change using a microplate reader. Thus, in one embodiment, the degree of nanoparticle aggregation is assessed by determining the % change in A_{534 nm} by detecting the change using a microplate reader. A_{534 nm} corresponds to the surface plasmon wavelength peak of the epitope-functionalized nanoparticles. Thus, measuring the percentage change relative to the initial absorption or fluorescence wavelength characteristic of the nanosensor allows the sensor response to be determined.

In various embodiments, the assay/method has high sensitivity and/or specificity. In various embodiments, the assay/method has a sensitivity of at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% or at least about 100%.

In various embodiments, the assay/method has a specificity of at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% or at least about 100%.

In one embodiment, the subject is a mammal, such as a monkey, rabbit, mouse, rat, pig or dog.

In one embodiment, the subject is a human.

In one embodiment, the degree of nanoparticle aggregation in a sample from a subject having a SARS-CoV-2 infection is more than the mean+1, 2 or 3 standard deviations, than the degree of nanoparticle aggregation in a control sample.

In one embodiment, the degree of nanoparticle aggregation in a sample from a subject having a SARS-CoV-2 infection is more than the mean+1 standard deviation than the degree of nanoparticle aggregation in a control sample. Thus, in one embodiment the % change in A_{534 nm} in a sample from a subject having a SARS-CoV-2 infection is more than the mean+1 standard deviations of the % change in A₅₃₄ nm in a control sample.

In one embodiment, the degree of nanoparticle aggregation in a sample from a subject having a SARS-CoV-2 infection is more than the mean+2 standard deviations than the degree of nanoparticle aggregation in a control sample. Thus, in one embodiment the % change in A₅₃₄ nm in a sample from a subject having a SARS-CoV-2 infection is more than the mean+2 standard deviations of the % change in A₅₃₄ nm in a control sample.

In one embodiment, the degree of nanoparticle aggregation in a sample from a subject having a SARS-CoV-2 infection is more than the mean+3 standard deviations of the degree of nanoparticle aggregation in a control sample. Thus, in one embodiment the % change in A_{534 nm} in a sample from a subject having a SARS-CoV-2 infection is more than the mean+3 standard deviations of the % change in A₅₃₄ nm in a control sample. Advantageously, the present inventors have established that this threshold provides an appropriate balance between sensitivity and specificity of the assay/method.

In one embodiment, a % change in A₅₃₄ nm of about 15 to 25% or more, such as 20% or more indicates that the sample is from a subject having a SARS-CoV-2 infection. In one embodiment, a % change in A₅₃₄ nm of about 15% or more, such as 15%, 16%, 17%, 18%, 19% or more indicates that the sample is from a subject having a SARS-CoV-2 infection. In one embodiment, a % change in A₅₃₄ nm of about 20% or more, such as 20%, 21%, 22%, 23%, 24% or more indicates that the sample is from a subject having a SARS-CoV-2 infection. In one embodiment, a % change in A₅₃₄ nm of about 25% or more, such as 25%, 26%, 27%, 28%, 29%, 30% or more indicates that the sample is from a subject having a SARS-CoV-2 infection.

In one embodiment, a % change in A₅₃₄ nm of about 15 to 25% or more, such as 20% or more indicates that the sample is from a subject having a SARS-CoV-2 infection. The present inventors have determined from clinical tests that a % change in A_{534 nm} greater than this threshold provides an appropriate balance between sensitivity and specificity of the assay/method. This threshold is thus suitable for establishing the status of a sample as COVID-19 positive or negative.

In one embodiment, the control sample is a sample from a subject that does not have a SARS-CoV-2 infection. In one embodiment, the control sample is a sample from a subject who has not previously had a SARS-CoV-2 infection.

In one embodiment, the subject identified as having a SARS-CoV-2 infection is treated with a therapeutic agent, such as an anti-viral agent. Thus, in one embodiment, the therapeutic agent is an anti-viral agent. In one embodiment, the therapeutic agent is selected from the group consisting of Paxlovid™ (nirmatrelvir and ritonavir), molnupiravir, fluvoxamine, sotrovimab, bebtelovimab, remdesivir, and a combination thereof.

Thus, in one embodiment, the subject identified as having a SARS-CoV-2 infection is treated with Paxlovid™. In one embodiment, the subject identified as having a SARS-CoV-2 infection is treated with molnupiravir. In one embodiment, the subject identified as having a SARS-CoV-2 infection is treated with fluvoxamine. In one embodiment, the subject identified as having a SARS-CoV-2 infection is treated with sotrovimab. In one embodiment, the subject identified as having a SARS-CoV-2 infection is treated with bebtelovimab. In one embodiment, the subject identified as having a SARS-CoV-2 infection is treated with remdesivir. In one embodiment, the subject identified as having a SARS-CoV-2 infection is treated with a combination of two or more of the abovementioned therapeutic agents.

### BRIEF DESCRIPTION OF FIGURES

FIG. 1A shows a diagram of the nanosensors of the present disclosure.

FIG. 1B shows a table indicating the characteristics of the SARS-CoV-2 B-cell linear peptide epitopes S14P5, S20P2, S21P2 and N4P5 of the present disclosure.

FIG. 1C shows a diagram illustrating the nanoparticle-based COVID-19 IgG assay/method of the present disclosure.

FIG. 2A shows the UV-vis spectra of the single peptide coated gold nanoparticles S14P5-AuNPs before and after addition of CoV-2 anti-S1 IgG in the range of 50-500 nM.

FIG. 2B shows the UV-vis spectra of the S14P5-AuNPs before and after addition of CoV-2 anti-S1 IgG in the range of 10-60 nM.

FIG. 2C shows the A_{353 nm}/A_{650 nm} ratio as a function of IgG concentration. Also shown are photos of the mixture indicating a visible colour change from red to purple (left to right) at IgG concentration of 100 nM or higher.

FIG. 2D shows a negative control of SARS anti-S1 and COVID-19 anti-S2, indicating the specificity of the S14P5-AuNPs to COVID-19 anti-S1 IgGs.

FIG. 3A shows the UV-vis spectra of the S20P2-AuNPs before and after addition of CoV-2 anti-S2 IgGs.

FIG. 3B shows the UV-vis spectra of the S21P2-AuNPs before and after addition of CoV-2 anti-S2 IgGs.

FIG. 4A shows the UV-vis spectra of the N4P5-AuNPs before and after addition of CoV-2 anti-N IgGs.

FIG. 4B shows the peak intensity change of the N4P5-AuNPs before and after addition of CoV-2 anti-N IgGs.

FIG. 4C shows a series of photos of the visual changes of the N4P5-AuNPs before and after addition of CoV-2 anti-N IgGs.

FIG. 5A shows the UV-vis spectra of the S14P5/S20P2 dual peptide-AuNPs before and after addition of individual and pooled IgGs.

FIG. 5B shows a diagram of the S14P5/S20P2 dual peptide-AuNPs.

FIG. 5C shows the peak intensity change of the S14P5/S20P2 dual peptide-AuNPs before and after addition of individual and pooled IgGs.

FIG. 6A shows the UV-vis spectra of the N4P5/S14P5 dual peptide-AuNPs before and after addition of individual and pooled IgGs.

FIG. 6B shows a diagram of the N4P5/S14P5 dual peptide-AuNPs.

FIG. 6C shows the peak intensity change of the N4P5/S14P5 dual peptide-AuNPs before and after addition of individual and pooled IgGs.

FIG. 7A shows the UV-vis spectra of S14P5 conjugated using thiol-AuNPs in water at various S14P5 peptide concentrations.

FIG. 7B shows the UV-vis spectra of S14P5 conjugated using thiol-AuNPs in 1×PBS buffer at various S14P5 peptide concentrations.

FIG. 7C shows the UV-vis spectra of S21P2 conjugated using thiol-AuNPs in water at various S21P2 peptide concentrations.

FIG. 7D shows the UV-vis spectra of S21P2 conjugated using thiol-AuNPs in 1×PBS buffer at various S21P2 peptide concentrations.

FIG. 8A shows the UV-vis spectra of S14P5 conjugated using thiol-AuNPs without the use of a spacer with addition of anti-COVID-19 S1 and S2 IgGs. S14P5-AuNPs were prepared from 100 nM peptide and 5 nM AuNP, stabilised in 0.5% Tween 20.

FIG. 8B shows the UV-vis spectra of S14P5/S21P2 conjugated using thiol-AuNPs without the use of a spacer with addition of anti-COVID-19 S1 and S2 IgGs.

FIG. 8C shows the UV-vis spectra of S14P5 conjugated using thiol-AuNPs with and without use of a spacer with addition of anti-COVID-19 IgGs.

FIG. 8D shows the UV-vis spectra of S21P2 conjugated using thiol-AuNPs with the use of a cysteine spacer vs the use of a CALNN spacer.

FIG. 9A shows the UV-vis spectra of S14P5 conjugated to a silver/gold nanoparticle alloy (Ag_{0.5}Au_{0.5}, 30 nM) via a strepatavidin-biotin interaction with addition of COVID-19 anti-S1 IgGs.

FIG. 9B shows the UV-vis spectra of S14P5 conjugated to a silver nanoparticle alloy (Ag, 10 nM) via a streptavidin-biotin interaction with addition of COVID-19 anti-S1 IgGs.

FIG. 9C shows the UV-vis spectra of S14P5 conjugated to a silver nanoparticle alloy (Ag, 10 nM) via a thiol-Ag interaction with the CALNN spacer upon addition of COVID-19 anti-S1 IgGs.

FIG. 10A shows the UV-vis spectra of S14P5-AuNP conjugated via a streptavidin-biotin interaction with addition of different concentrations of COVID-19 IgGs.

FIG. 10B shows the UV-vis spectra of S20P2-AuNP conjugated via a streptavidin-biotin interaction with addition of different concentrations of COVID-19 IgGs.

FIG. 10C shows the UV-vis spectra of N4P5-AuNP conjugated via a streptavidin-biotin interaction with addition of different concentrations of COVID-19 IgGs.

FIG. 10D shows a graph of the % sensor response for N4P5-AuNP, S14P5-AuNP and S20P2-AuNP vs COVID-19 gG concentration.

FIG. 10E shows a heatmap comparing the sensor response across the different nanoparticle-IgG combinations.

FIG. 11A shows the adjusted sensorgram for the S14N5 peptide determined by Biacore T200.

FIG. 11B shows the adjusted sensorgram for the S21P2 peptide determined by Biacore T200.

FIG. 11C shows the adjusted sensorgram for the S20P2 peptide determined by Biacore T200.

FIG. 11D shows the adjusted sensorgram for the N4P5 peptide determined by Biacore T200.

FIG. 11E shows a table of the Kd of the peptides of the present disclosure determined by Biacore T200.

FIG. 12A shows a schematic depiction of surface plasmon resonance (SPR) spectroscopy.

FIG. 12B shows a schematic depiction of the fluorescence polarization (FP) working principle to characterise the presently disclosed peptide epitopes' binding affinity and specificity towards SARS-CoV-2 IgG.

FIG. 12C shows SPR sensorgram data showing real-time binding of anti-SARS-CoV-2 S1 IgG at varying concentrations to the immobilized S14P5 epitope. Dashed line represents model fit.

FIG. 12D shows a graph indicating the Relative FP increase when 100 nM peptide epitopes were treated with 200 nM SARS-CoV-2, SARS-CoV, and normal human IgGs. Two-tailed unpaired t-tests, ****P<0.0001, ***P<0.001, **P<0.01, *P<0.05, ns=not significant, compared with SARS-CoV-2 IgG treatment for each epitope. Data represent mean±standard deviation from 3 independent experiments.

FIG. 13A shows a schematic representation of the nanosensor detection mechanism, which relies on the aggregation of epitope-tagged AuNPs induced by binding to the cognate paratopes of SARS-CoV-2 IgG antibodies.

FIG. 13B shows the UV-vis spectra of AuNPs upon conjugation of SA and subsequently the biotinylated SARS-CoV-2 epitope peptide S14P5, which was used as a representative epitope. The red-shift in the peak absorbance after consecutive AuNP functionalization with SA and the linear epitope is highlighted in the inset.

FIG. 13C shows a graph of the hydrodynamic size of AuNPs before and after functionalization with SA and individual linear SARS-CoV-2 epitopes as measured with DLS. Data represent mean±standard deviation from 3 independent samples.

FIG. 14A shows the absorption spectra of S14P5-AuNPs upon treatment with anti-SARS-CoV-2 S1 IgG of varying concentrations.

FIG. 14B shows a photo demonstrating the Visual detection of antibody-induced aggregation of S14P5-AuNPs taken 30 min after anti-SARS-CoV-2 S1 IgG introduction.

FIG. 14C shows TEM images of S14P5-AuNPs after the addition of pure PBS or 100 nM anti-SARS-CoV-2 S1 IgG. Scale bars: 100 nm.

FIG. 14D shows a graph of the Temporal profile of the plasmon peak decrease when challenged with IgGs of SARS-CoV-2, SARS-CoV, and normal human. Measurements were taken at 10 min intervals for 60 min. Data represent mean±standard deviation from 3 independent experiments.

FIG. 14E shows the dose-dependent nanosensor response curves for the four single epitope-functionalized AuNPs. Data were fit with the Hill binding model. Data represent mean±standard deviation from 3 independent experiments.

FIG. 14F shows a graph indicating the selectivity of epitope-tagged AuNPs against closely related human coronaviruses. Two-tailed unpaired t-tests, ****P<0.0001, ***P<0.001, **P<0.01, *P<0.05, ns=not significant, compared with SARS-CoV-2 antibody treatment for each epitope. Data represent mean±standard deviation from 3 independent experiments.

FIG. 15A shows a heat map of nanosensor responses for different single- and dual-epitope-functionalized AuNPs against varying concentrations of respective SARS-CoV-2 IgGs.

FIG. 15B shows a graph indicating the Limit of detection (LOD) of single- and dual-epitope-functionalized AuNPs when challenged with anti-S1, anti-S2, or anti-N IgGs of SARS-CoV-2.

FIG. 15C shows a graph indicating the relative changes in plasmon peak intensity of nonpassivated and PEG-passivated AuNPs, functionalized with S14P5 alone or both S14P5 and S21P2 epitopes, against 100 nM anti-SARS-CoV-2 S1 IgG in different final concentrations of human plasma. Two-tailed unpaired t-tests, ***P<0.001, **P<0.01, *P<0.05. Data represent mean±standard deviation from 3 independent experiments.

FIG. 16A shows a graph demonstrating the detection of detection of SARS-CoV-2 antibodies in clinical samples with PEG-passivated S14P5-AuNPs.

FIG. 16B shows a graph demonstrating the detection of detection of SARS-CoV-2 antibodies in clinical samples with PEG-passivated S14P5/S21P2-AuNPs.

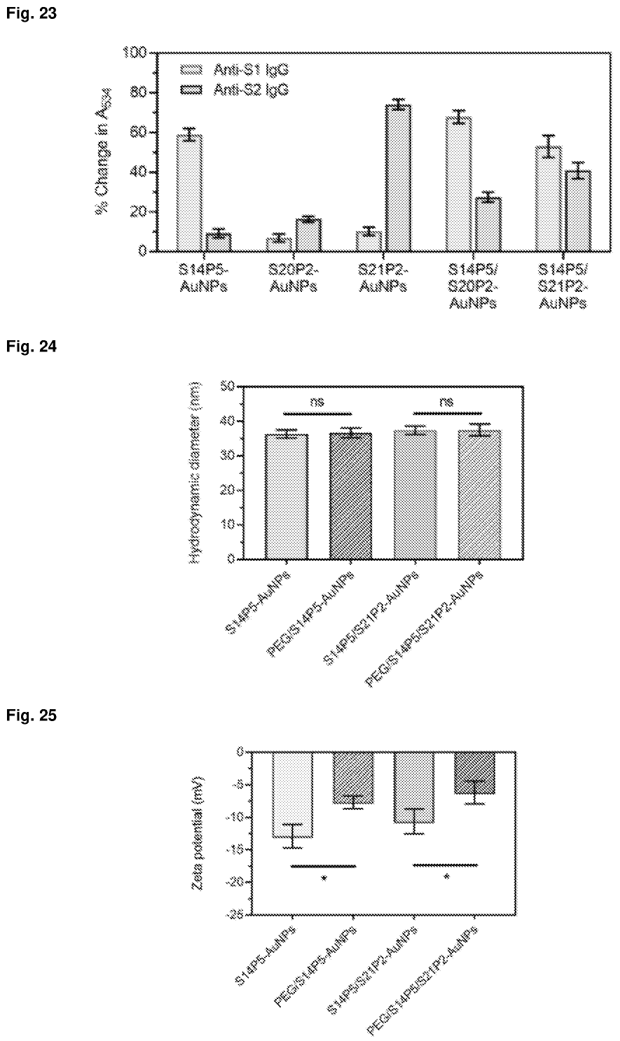

For FIG. 16A and FIG. 16B, both nanosensors were challenged with plasma fractions obtained from 10 healthy donors and 10 COVID-19-infected patients of different clinical severity. Thresholds obtained from the validation test were then applied in a blind test where both nanoparticles were used to detect SARS-CoV-2 IgG from clinical samples consisting of 2 healthy donors and 13 COVID-19-infected patients of different severity. OH=old healthy, YH=young healthy, AS=asymptomatic, MI=mild, MO=moderate, and SV=severe. Dashed line and green-shaded region represent threshold cutoff obtained from the mean+3 standard deviations of healthy patient samples in a validation test (n=10).

FIG. 17A shows the SPR sensorgram data showing real-time interaction between immobilized S20P2 peptide epitopes with varying concentrations of epitope-specific SARS-CoV-2 IgG anti-S2 IgGs. Dashed line represents model fit.

FIG. 17B shows the SPR sensorgram data showing real-time interaction between immobilized S21P2 peptide epitopes with varying concentrations of epitope-specific SARS-CoV-2 IgG anti-S2 IgGs. Dashed line represents model fit.

FIG. 17C shows the SPR sensorgram data showing real-time interaction between immobilized N4P5 peptide epitopes with varying concentrations of epitope-specific SARS-CoV-2 IgG anti-N IgGs. Dashed line represents model fit.

FIG. 18A shows the steady-state SPR analysis of binding between S14P5 epitopes with different concentrations of epitope-specific SARS-CoV-2 anti-S1 IgGs. Dashed line represents model fit.

FIG. 18B shows the steady-state SPR analysis of binding between S20P2 epitopes with different concentrations of epitope-specific SARS-CoV-2 anti-S2 IgGs. Dashed line represents model fit.

FIG. 18C shows the steady-state SPR analysis of binding between S21P2 epitopes with different concentrations of epitope-specific SARS-CoV-2 anti-S2 IgGs. Dashed line represents model fit.

FIG. 18D shows the steady-state SPR analysis of binding between N4P5 epitopes with different concentrations of epitope-specific SARS-CoV-2 anti-N IgGs. Dashed line represents model fit.

FIG. 19 shows a graph of the zeta potential of citrate-capped AuNPs before and after successive streptavidin functionalization and epitope tagging. Data represent mean±s.d. from 3 independent experiments.

FIG. 20A shows the results of the salt-induced aggregation test of citrate-AuNPs. Experiment was conducted in 1×PBS.

FIG. 20B shows the results of the salt-induced aggregation test of SA-AuNPs. Experiment was conducted in 1×PBS.

FIG. 20C shows the results of the salt-induced aggregation test of S14P5-AuNPs. Experiment was conducted in 1×PBS.

FIG. 20D shows the results of the salt-induced aggregation test of S20P2-AuNPs. Experiment was conducted in 1×PBS.

FIG. 20E shows the results of the salt-induced aggregation test of S21P2-AuNPs. Experiment was conducted in 1×PBS.

FIG. 20F shows the results of the salt-induced aggregation test of N4P5-AuNPs. Experiment was conducted in 1×PBS.

FIG. 21 shows the absorption spectra of S14P5-AuNPs upon treatment with PBS, 100 nM of SARS-CoV-2 IgG, and control IgGs of SARS-CoV and normal human.

FIG. 22A shows the absorption spectra of S20P2-AuNPs upon the introduction of respective SARS-CoV-2 IgG at different concentrations.

FIG. 22B shows the absorption spectra of S21P2-AuNPs upon the introduction of respective SARS-CoV-2 IgG at different concentrations.

FIG. 22C shows the absorption spectra of N4P5-AuNPs upon the introduction of respective SARS-CoV-2 IgG at different concentrations.

FIG. 23 shows a graph of the relative changes in plasmon peak intensity of single epitope- and dual epitope-functionalized AuNPs upon treatment with 50 nM of anti-S1 IgG or anti-S2 IgG of SARS-CoV-2. Data represent mean±s.d. from 3 independent experiments.

FIG. 24 shows a graph of the hydrodynamic diameter of S14P5-AuNPs, S14P5/S21P2-AuNPs, and their PEG-passivated counterparts. Two-tailed unpaired t-tests, ns=not significant. Data represent mean±s.d. from 3 independent experiments.

FIG. 25 shows a graph of the zeta potential of S14P5-AuNPs, S14P5/S21P2-AuNPs, and their PEG-passivated counterparts. Two-tailed unpaired t-tests, *P<0.05. Data represent mean±s.d. from 3 independent experiments.

### EXAMPLES

Example embodiments of the disclosure will be better understood and readily apparent to one of ordinary skill in the art from the following discussions and if applicable, in conjunction with the figures. It should be appreciated that other modifications may be made without deviating from the scope of the invention. Example embodiments are not necessarily mutually exclusive as some may be combined with one or more embodiments to form new exemplary embodiments. The example embodiments should not be construed as limiting the scope of the disclosure.

### Example 1—Single Peptide (S14P5) Coated AuNPs for COVID-19 Anti-S1 IgGs

Citrate-capped AuNP (5 nM) was functionalized with thiol labeled S14P5 peptide (10-1000 nM). Thiol-labeled short peptide (CALNN, 1-10 μM) was used to block the un-covered surface. The peptide conjugation and blocking were performed by incubation of the peptides for 15 min. Tween 20 (0.01%-10%) was added to stabilize the conjugates before adding the appropriate amount of 10×PBS to reach a final concentration of 1×PBS. The obtained peptide-AuNPs was separated (via centrifugation for 10 min, at 14000RCF) from the unreacted peptides. The conjugates were reconstituted using 1×PBS buffer (0.01 M phosphate buffer, 0.0027 M potassium chloride and 0.137 M sodium chloride, pH 7.4, at 25° C.) and 0.5% v/v Tween20. The resulting AuNPs were washed again via centrifugation as per the previous step and reconstituted at half of the original volume to produce an AuNP solution of approximately 10 nM AuNP in 1×PBS and 0.5% v/v Tween20.

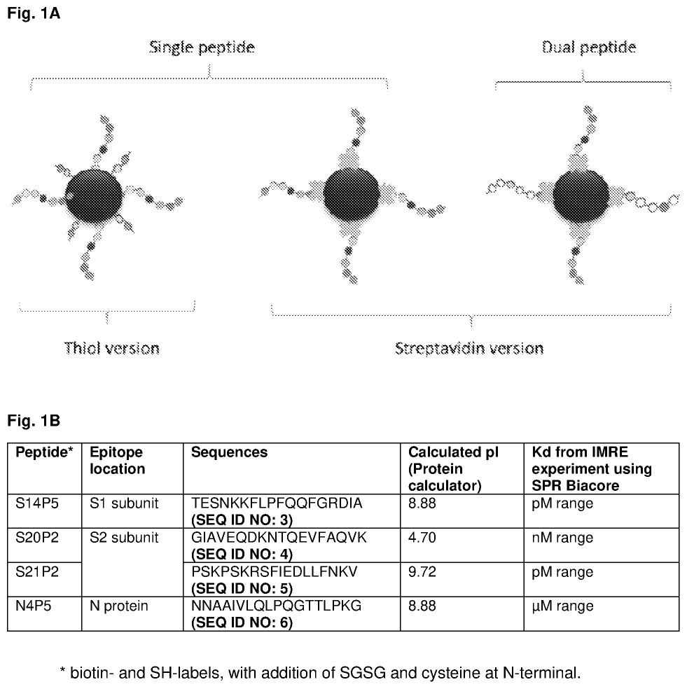

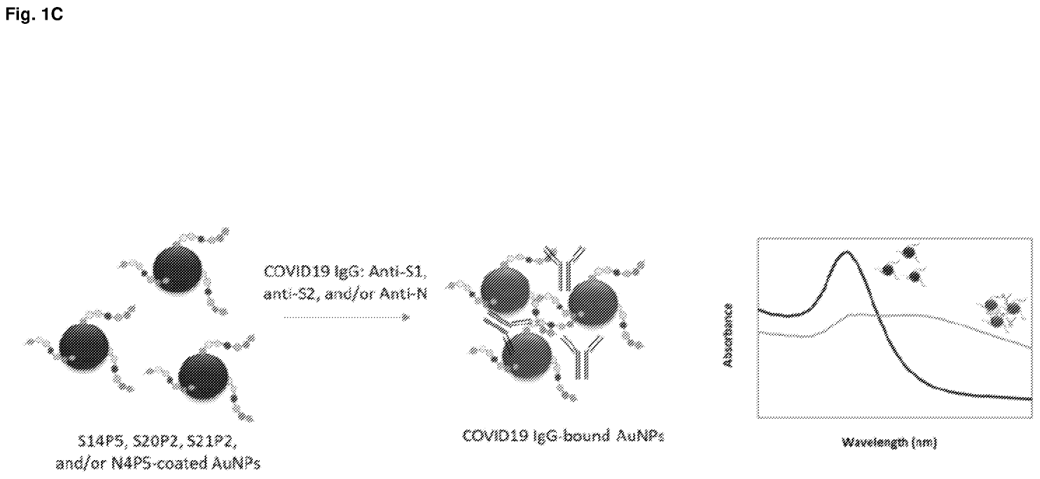

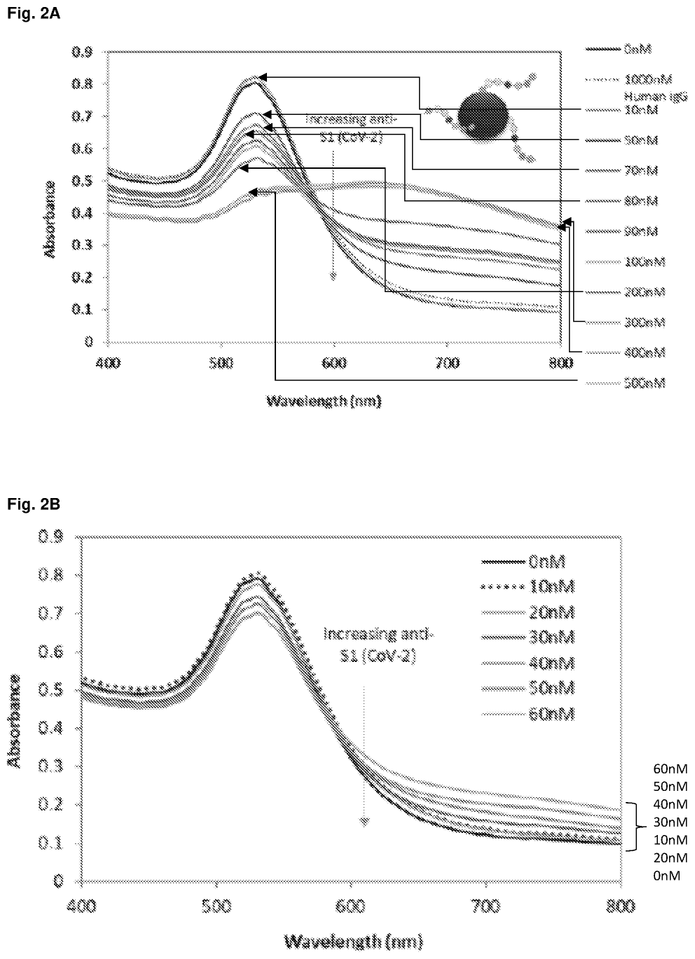

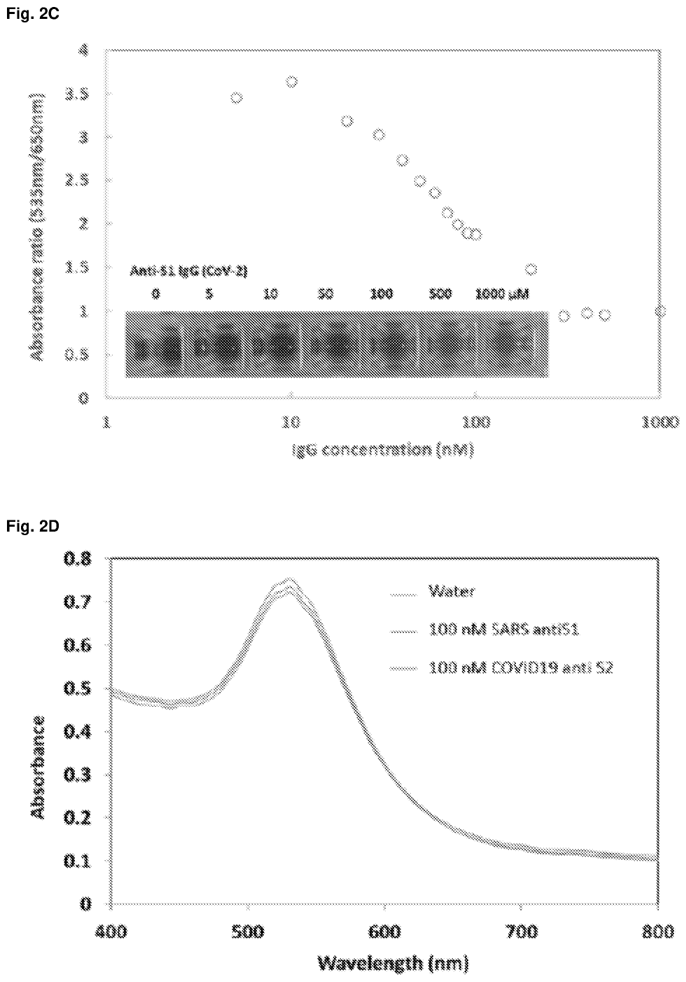

For IgG test, COVID19 anti-S1 IgG (SINO Biological) of varied concentrations was mixed with the S14P5-AuNPs in 384 (or 96) well plate, and incubated for 15 min. particularly, up to 20 μl of IgG solution was mixed with 5 μl of 10×PBS and added to 25 μl of the above solution in a 384 well plate. The mixture was allowed to incubate for 15 minutes at room temperature. UV-vis spectra before and after IgG addition, over a range of 10-500 nM are recorded (FIGS. 2A and 1B). Human IgG (1000 nM) was used as negative control to confirm the selectivity to CoV-2 anti-S1 IgG. SARS anti-S1 and COVID19 anti-S2 are also tested (FIG. 2C) to prove the selectivity of the S14P5-AuNP to only the anti-S1 of COVID19. It is very obvious that COVID19 anti-S1 can cause the particles to aggregate, presumably due to the crosslinking binding of the IgG to the peptides on the AuNPs surface. A visible change in colour of the mixture from red to purple can be observed for IgG concentrations at 100 nM or higher (photo in FIG. 2C). The degree of aggregation, measured using the absorbance ratio of A353 nm/A650 nm (FIG. 2D), is related the IgG concentration; the linear range is 10-100 nM (1.5 μg/ml-15 μg/ml) and LOD is ˜30 nM (4.5 μg/ml). This is suitable to detect the onset of an Covid19 infection. Anti Covid19 IgG concentrations reach a peak during 21-25 days after infection at a concentration of approximately 16.5 μg/mL of reactive IgG. The concentration of reactive IgG stays relatively high (11.4 μg/mL) until 41 days from the date of infection.

### Example 2—Single Peptide (S20P2 or S21P2) Coated AuNPs for COVID-19 Anti-S2 IgGs

Citrate-coated AuNP was functionalized with streptavidin (SA) by mixing 500 nM of SA with 8 nM of citrate-coated AuNP. The mixture was incubated at room temperature for 30 min, and unbound SA was removed from the SA-coated AuNPs (SA-AuNPs) with centrifugation at 12,500 RCF for 15 mins. Then, biotinylated S20P2 or S21P2 peptide (10 or 1 μM) was added to conjugate to the SA-AuNPs. Unreacted peptides were removed from the peptide-AuNP conjugates with centrifugation at 12500 RCF for 12 mins. The conjugates were reconstituted into ultrapure water for further tests with IgG.

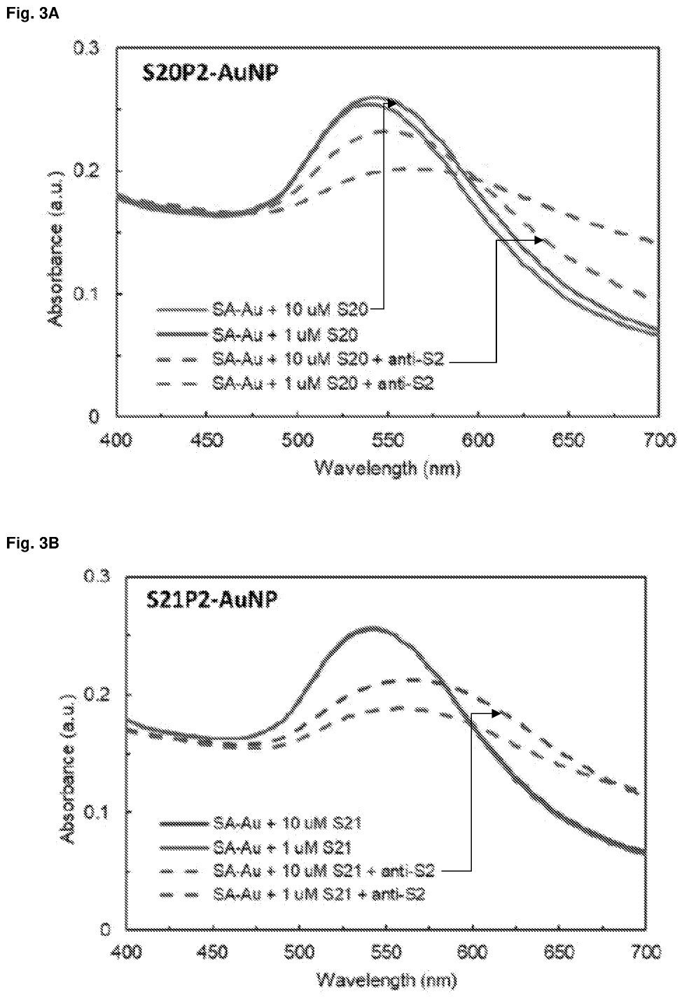

For IgG test, 20 μL of COVID19 anti-S2 IgG (SINO Biological) was added to 80 μL of peptide-AuNP conjugates in a 96-well plate. UV-vis spectrum before and after IgG addition are recorded (FIGS. 3A and B). For both S20P2- and S21P2-conjugates, COVID19 anti-S2 IgG can cause significant aggregation of peptide-coated AuNPs. For both conjugates, a higher degree of aggregation is observed when lower peptide concentration (1 μM) is used, presumably due to the diluted coating density around AuNP which poses less steric hindrance for binding with IgG.

### Example 3—Single Peptide (N4P5) Coated AuNPs for COVID19 Anti-N IgG

Citrate-coated AuNP was functionalized with streptavidin (SA) by mixing 500 nM of SA with 8 nM of citrate-coated AuNP. The mixture was incubated at room temperature for 30 min, and unbound SA was removed from the SA-coated AuNPs (SA-AuNPs) with centrifugation at 12,500 RCF for 15 mins. Then, biotinylated N4P5 peptide (1 μM) was conjugated to the SA-AuNPs. Unreacted peptides were removed from the peptide-AuNP conjugates with centrifugation at 12,500 RCF for 12 mins. The conjugates were reconstituted into ultrapure water for further tests with IgG.

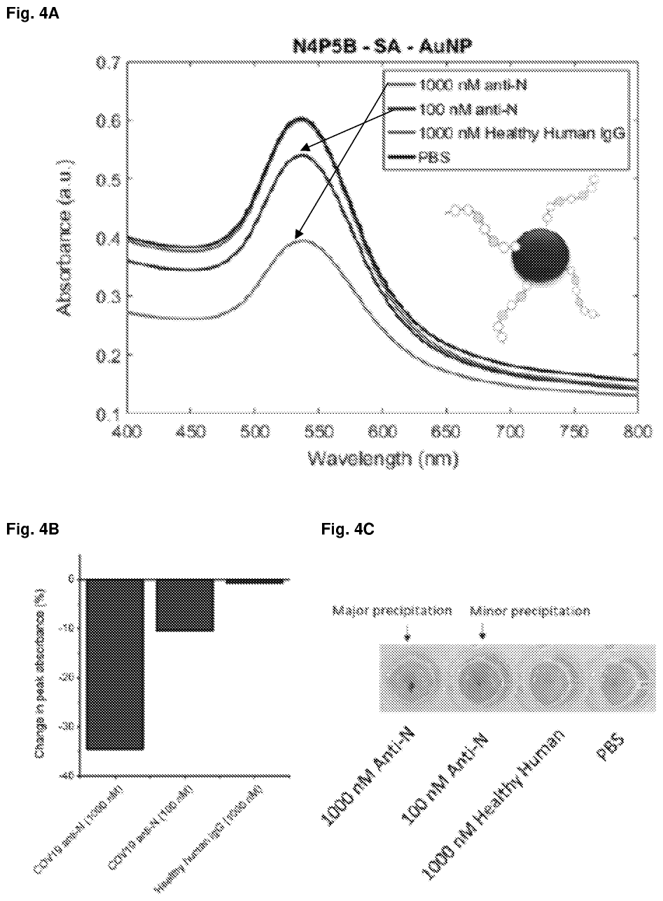

For IgG test, COVID19 anti-N IgG (SINO Biological) (100 and 1000 nM) was mixed with the peptide-AuNPs conjugate in 96-well plate, and incubated for 15 min at room temperature. In particular, 20 μL of IgG was added to 80 μL of peptide-AuNP conjugates in a 96-well plate. After incubation, 30 μL solution (excluding the precipitate) was transferred from each well to a 384-well plate for absorbance analysis to measure the non-precipitated peptide-AuNP concentration. UV-vis spectrum before and after IgG addition are recorded (FIG. 4A). Human IgG (1000 nM) was used as negative control to confirm the selectivity. COVID19 anti-N IgG can cause AuNPs to aggregate in a concentration-dependent manner, as seen by the degree of peak intensity drop (FIG. 4B). Furthermore, the AuNPs solutions show obvious precipitation due to specific IgG binding, which can be visualized with naked eyes, with major (1000 nM IgG) and minor (100 nM IgG) precipitation; whereas no precipitation is observed for the human IgG control and buffer control (FIG. 4C).

### Example 4—Binary/Dual Peptide (S14P5/S20P2) Coated AuNP for COVID19 Anti-S IgGs

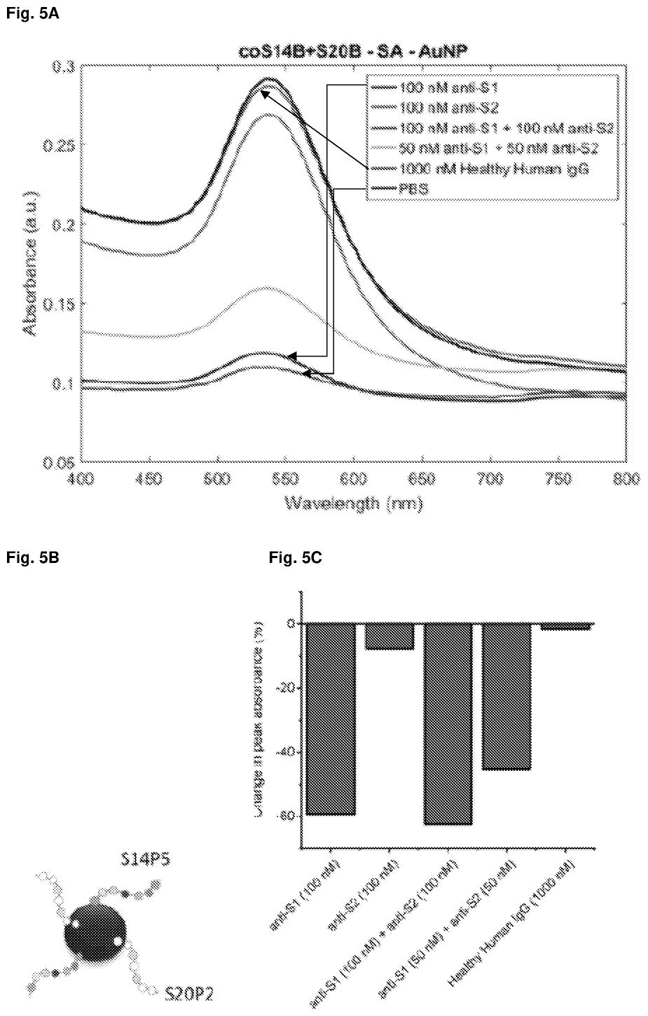

Citrate-coated AuNP was functionalized with streptavidin (SA) by mixing 500 nM of SA with 8 nM of citrate-coated AuNP. The mixture was incubated at room temperature for 30 min, and unbound SA was removed from the SA-coated AuNPs (SA-AuNPs) with centrifugation at 12,500 RCF for 15 mins. A mixture of 500 nM biotinylated S14P5 and 500 nM biotinylated S20P2 is added into the obtained SA-AuNP (5-10 nM). After 20 min incubation, the mixture is centrifuged at 12,500 RCF for 15 min to remove the unbound peptides. The dual peptide-AuNPs is challenged by COVID19 anti-S1, anti-S2, and a mixture of anti-S1 and anti-S2 IgG, respectively (FIG. 5A to C). The dual peptide-AuNPs are aggregated in response to individual IgG, with sensitivity trend following that of preliminary peptide binding characterization, i.e. S14P5 being much stronger than S20P2 in terms of binding to individual IgG). It is also responsive to the pooled COVID19 anti-S1 and anti-S2 mixture, with synergistic effect from each peptide-IgG binding as shown by the improved detection towards pooled IgG compared to individual IgG. The binary peptide-AuNP conjugates did not show any response towards human IgG.

### Example 5—Binary/Dual Peptide (N4P5/S14P5) Coated AuNP for COVID19 Anti-N and Anti-S IgGs

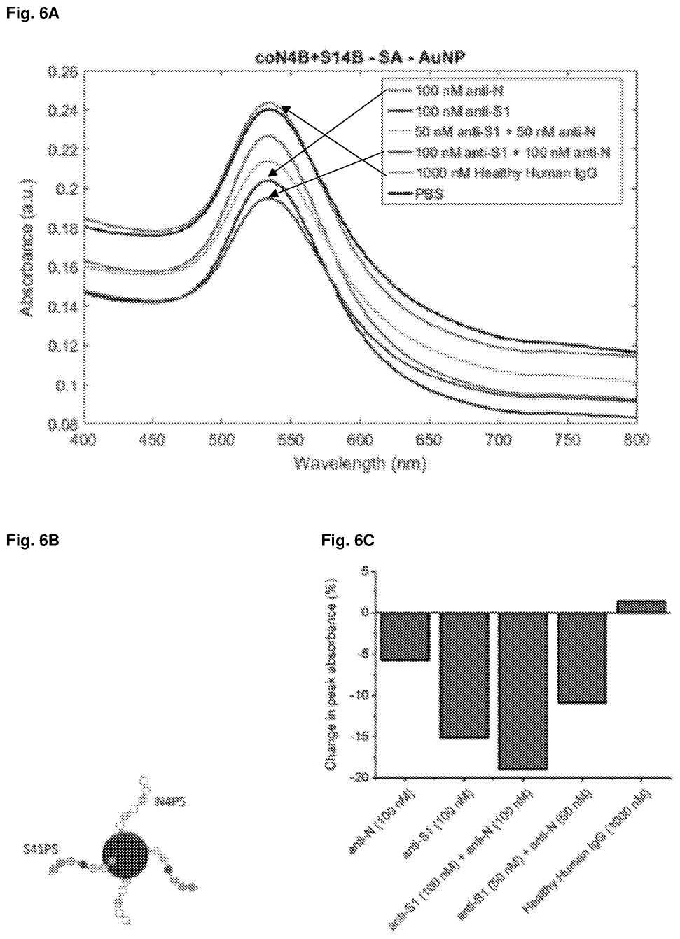

Citrate-coated AuNP was functionalized with streptavidin (SA) by mixing 500 nM of SA with 8 nM of citrate-coated AuNP. The mixture was incubated at room temperature for 30 min, and unbound SA was removed from the SA-coated AuNPs (SA-AuNPs) with centrifugation at 12,500 RCF for 15 mins. A mixture of 500 nM biotinylated N4P5 and 500 nM biotinylated S14P5 is added into the obtained SA-AuNP (5-10 nM). After 20-min incubation, the mixture is centrifuged at 12,500 RCF for 15 min to remove the unbound peptides. The dual peptide-AuNPs is challenged by COVID19 anti-N, anti-S1, and a mixture of anti-N and anti-S1 IgG, respectively (FIG. 6A to C). The dual peptide-AuNPs are aggregated in response to individual IgG, with sensitivity trend following that of preliminary peptide binding characterization, i.e. S14P5 being much stronger than N4P5 in terms of binding to individual IgG). This mirrors the trend presented in Example 4 (for S14P5/S20P2 binary peptide). It is also responsive to the pooled COVID19 anti-N and anti-S1 mixture, with synergistic effect from each peptide-IgG binding as shown by the improved detection towards pooled IgG compared to individual IgG. The binary peptide-AuNP conjugates did not show any response towards human IgG.

### Example 6—Investigation of Technical Difficulties in Conjugating SARS-CoV-2 Peptide Epitopes to AuNP

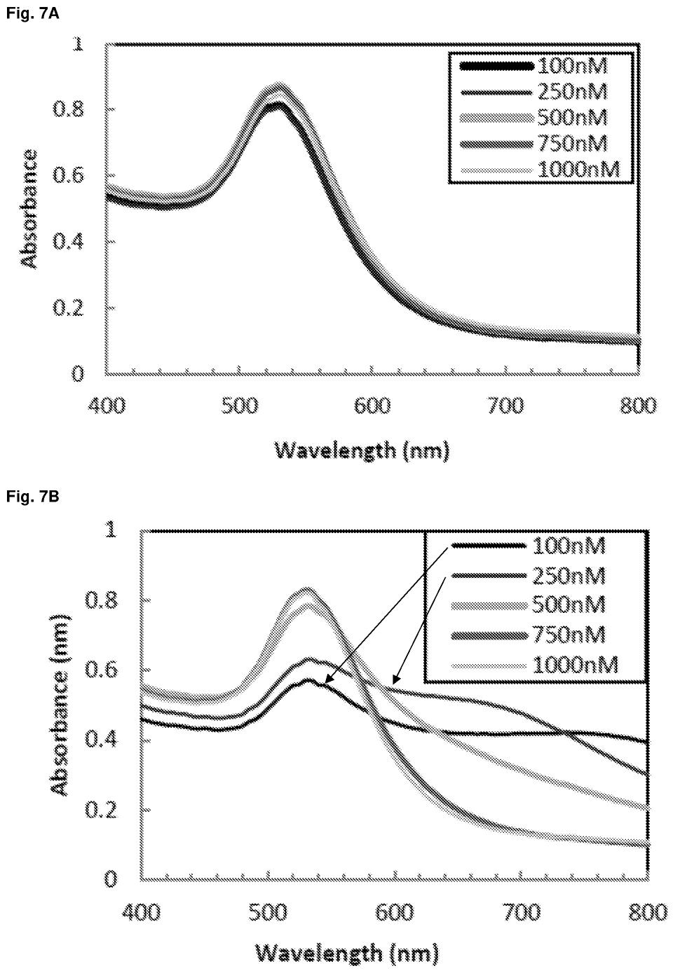

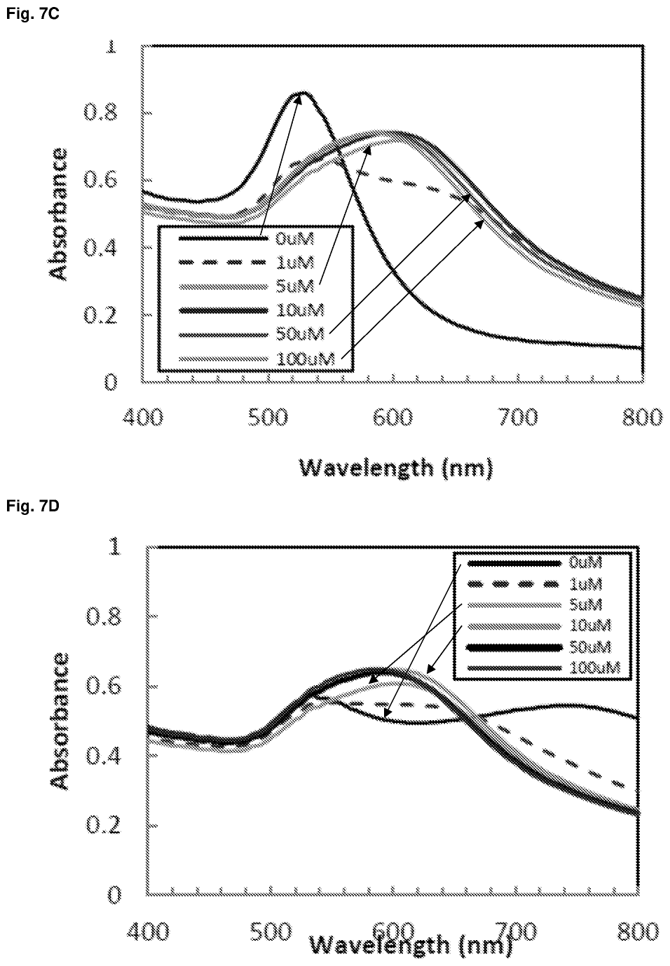

Attempts to conjugate the S21P2 peptide epitope using thiol-AuNPs interaction failed (See FIGS. 7C and D). Different concentrations of S21P2 were tested (up to 100 μM) but regardless of the concentration used, salt induced aggregation of the AuNPs was observed. However, when the streptavidin-biotin approach was attempted instead, this resulted in the successful conjugation of the S21P2 epitope to the AuNPs. See Example 2 above.

In comparison, for the S14P5 epitope, the conjugation via the thiol-AuNP interaction was successful and the resulting S14P5-AuNPs were stable in water (at nM peptide concentrations, see FIG. 7A) and in PBS buffer (at 750 nM and above, see FIG. 7B). In particular, the retained stability of S14P5-AuNP in PBS provides evidence of the successful conjugation, i.e., the peptide coating stabilized the AuNP against salt induced aggregation.

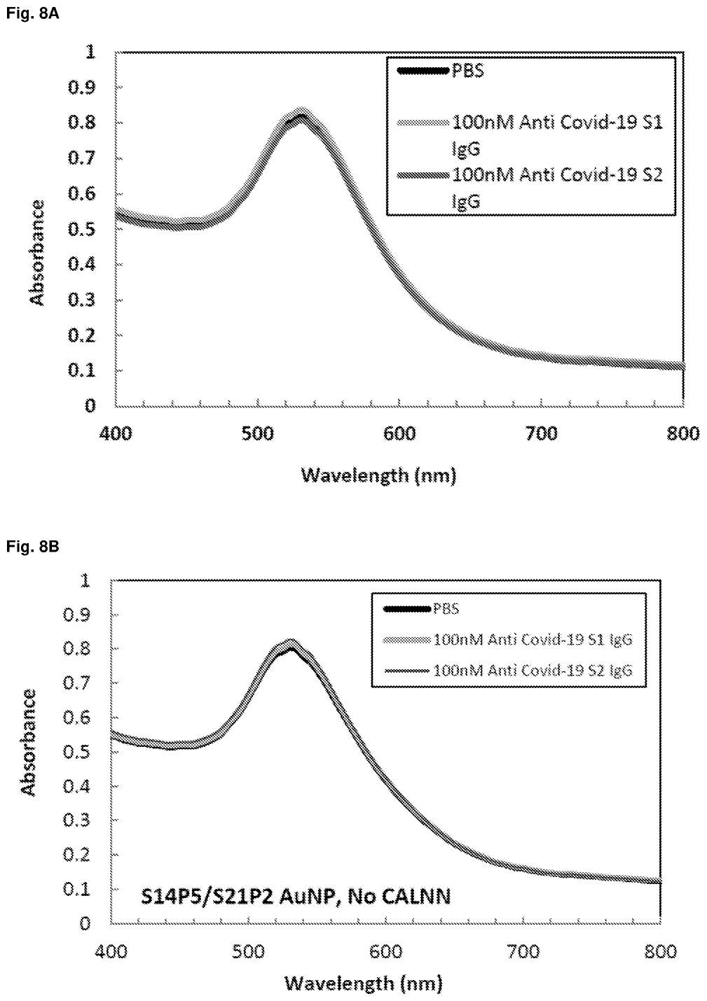

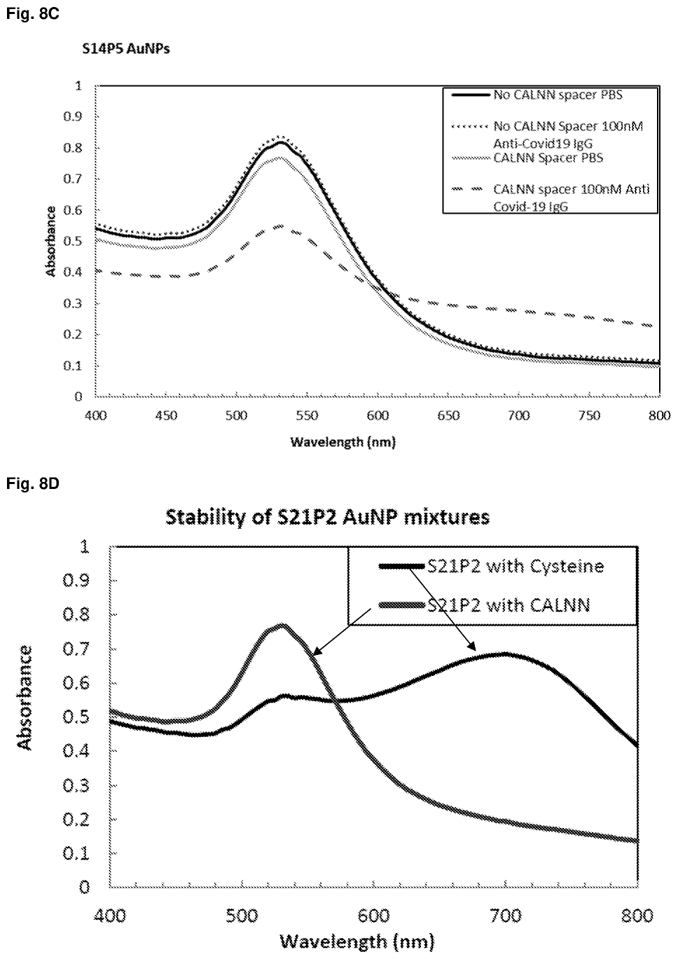

Unfortunately, although the S14P5-AuNP conjugation via the thiol-AuNP interaction was successful, subsequent experiments demonstrated that the S14P5-AuNPs were unable to bind to anti COVID-19 IgGs. See FIG. 8A. Specifically, the test against COVID-19 anti-S1 IgG (100 nM) failed to result in nanoparticle aggregation—the UV-vis profile was the same as that of the S14P2-AuNP with PBS and with anti-S2 IgG (which is a negative control since S14P5 is a S1 epitope).

A further attempt was made to conjugate the S21P2 epitope to the AuNPs via the thiol-AuNP approach. Since the S14P5 epitope could be successfully conjugated using this approach, an attempt was made to perform a dual conjugation using the S21P2 and S14P5 epitopes. Surprisingly, the dual conjugation of S14P5 (500 nM), followed by S21P2 (500 nM) was successful, as observed from the stable nanoparticles in PBS (see the black curve in FIG. 8B). Unfortunately, as was with the S14P5-AuNPs, the S14P5/S21P2 dual peptide AuNPs were also unable to respond to COVID-19 anti-S1 and anti-S2 IgGs. See FIG. 8B.

The present inventors surmised that the failure of the nanoparticles to respond to IgG could be due to insufficient separation between the peptide epitopes on the nanoparticle surface, thereby hindering the ability of the COVID-19 IgG molecules to bind to the conjugated epitopes. To address this potential issue, the inventors re-attempted the S21P2 and S14P5 single epitope thiol-AuNP conjugations, this time introducing a thiolyated spacer CALNN to occupy the empty space on the nanoparticles, thereby helping to separate the peptide epitopes and to also block the free surface of the nanoparticles. This approach was successful for both S14P5 and S21P2, and resulted in nanoparticle aggregation in response to COVID-19 IgGs. See FIGS. 8C and 8D. FIG. 8C shows the effects of a CALNN spacer on the responsiveness of the S14P5 AuNP to Anti COVID-19 S1 IgGs. Note that nanoparticle aggregation and the corresponding reduction in absorbance at 530 nm in the presence of the anti-COVID-19 IgGs was only observed when the CALNN spacer is used in conjunction with S14P5 conjugation.

The present inventors subsequently attempted to use cysteine as an alternative spacer based on the premise that a thiol group is required to form a chemical bond with gold or silver nanoparticles and that cysteine is the only naturally occurring sulphur/thiol containing amino acid. The use of cysteine as a spacer was however unsuccessful. FIG. 8D shows that the S21P2-AuNPs with cysteine spacer aggregated in the presence of Tween 20, even without PBS.

In summary, this Example demonstrates how the Thiol-metal interaction with spacer approach can be successfully used for producing the peptide-conjugated nanoparticles of the present disclosure.

### Example 7—Investigation of Suitability of Different Metal Nanoparticles

To investigate the possibility of employing other metal nanoparticles besides gold nanoparticles, a series of experiments using silver and gold/silver alloy nanoparticles was performed.

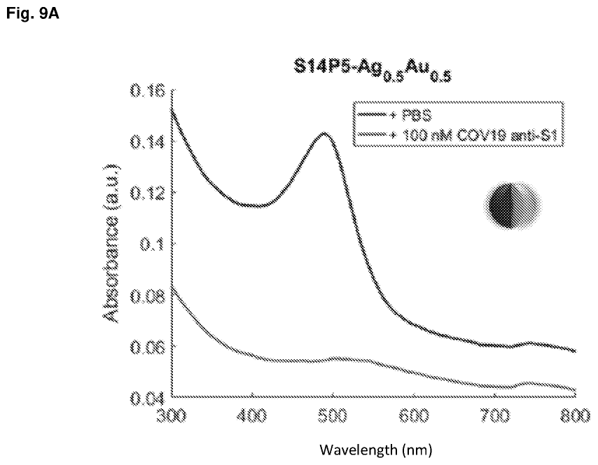

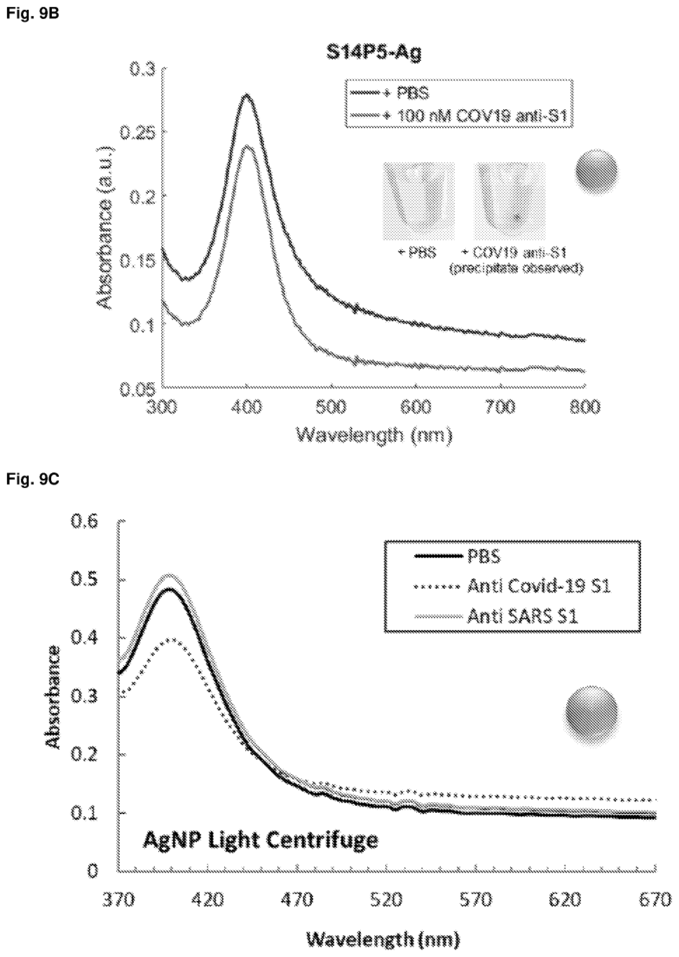

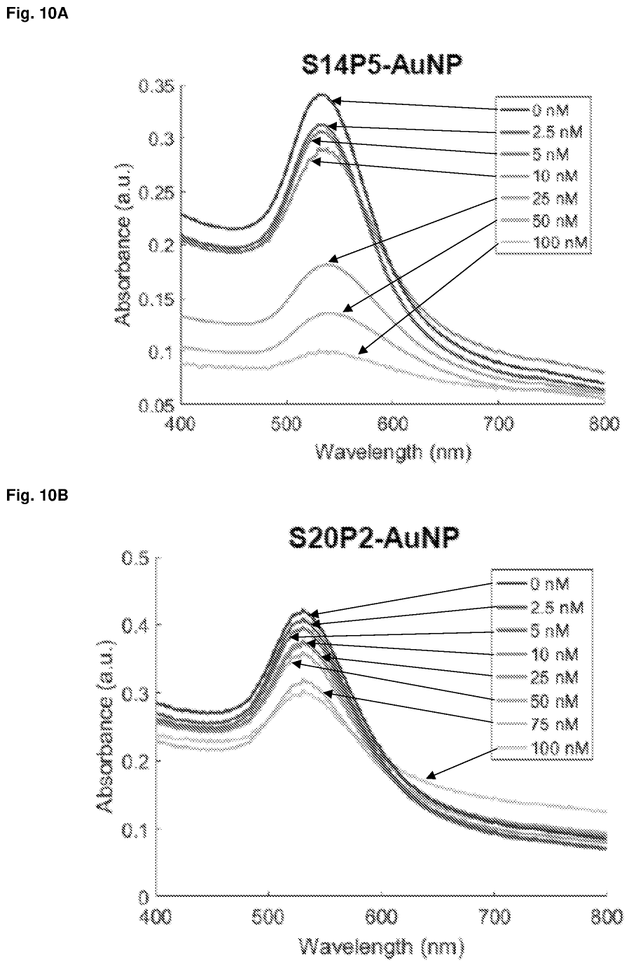

The S14P5 epitope was conjugated onto silver/gold nanoparticle alloy (Ag_{0.5}Au_{0.5}, 30 nm) and silver nanoparticles (AgNP, 10 nm) through the streptavidin-biotin approach (See FIGS. 9A and B). Both Ag/Au and Ag nanoparticles showed a decrease in absorbance upon the introduction of COV19 anti-S1 IgG. In addition, visible nanoparticle precipitation was observed for S14P5-Ag upon the introduction of COV19 anti-S1 IgGs.

The S14P5 epitope was also conjugated to the AgNP using the thiol-approach with the CALNN spacer (see FIG. 9C). As can be seen from FIG. 9C, the nanoparticles were responsive to anti-S1 IgG with the assistant of brief spinning (1 min using a portable centrifuge).

Thus, this Example demonstrates that alternative metal nanoparticles, other than gold nanoparticles can be successfully used for producing the peptide-conjugated nanoparticles of the present disclosure.

### Example 8—Determination of Full Range Concentration Response of Gold Nanoparticles for Detection of SARS-CoV-2 IgG Antibodies

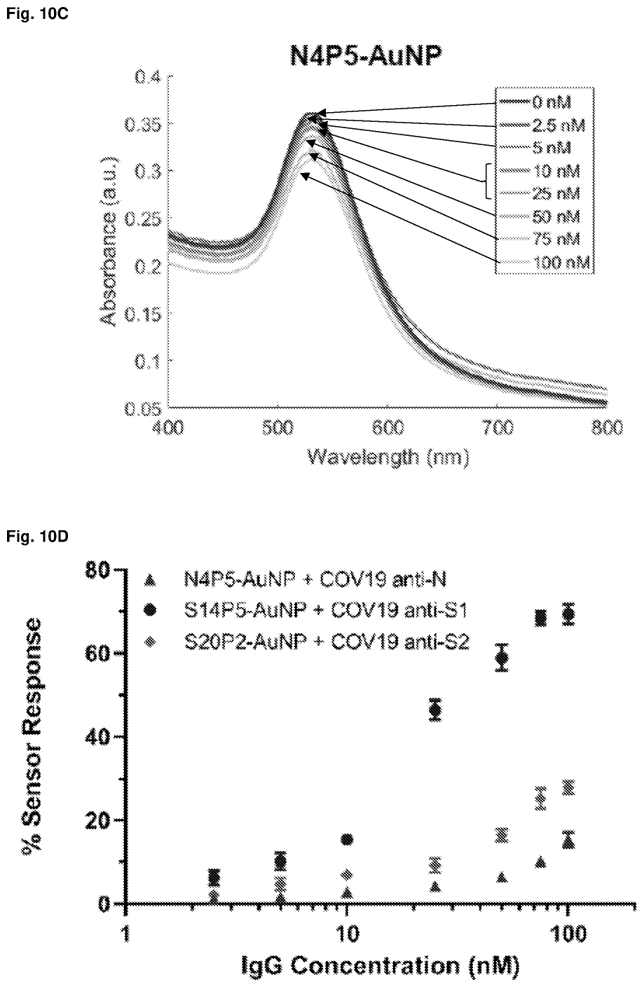

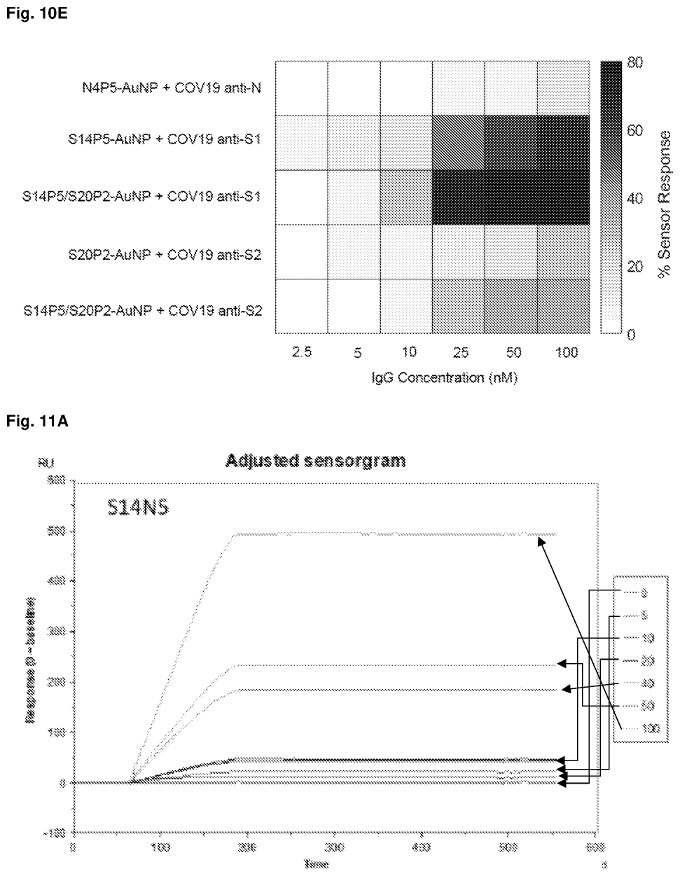

The Biacore T200 was used to determine the binding affinity trends of the various peptide conjugated AuNPS. The results of the analysis are shown in FIG. 10A to E. In particular, FIG. 10E shows a heatmap comparison of the sensor response across all the different nanoparticle-IgG combinations. In general, the order of sensitivity in descending order is: detection of anti-S1 IgGs>detection of anti-S2 IgGs>detection of anti-N IgGs. Interestingly, the S14P5/S20P2 dual peptide AuNPs+COVID-19 anti-S1 combination seems to be the most sensitive amongst the combinations tested.

### Example 9—Determination of Binding Affinity of the S14P5, S21P2, S20P2 and N4P5 Peptide Epitopes

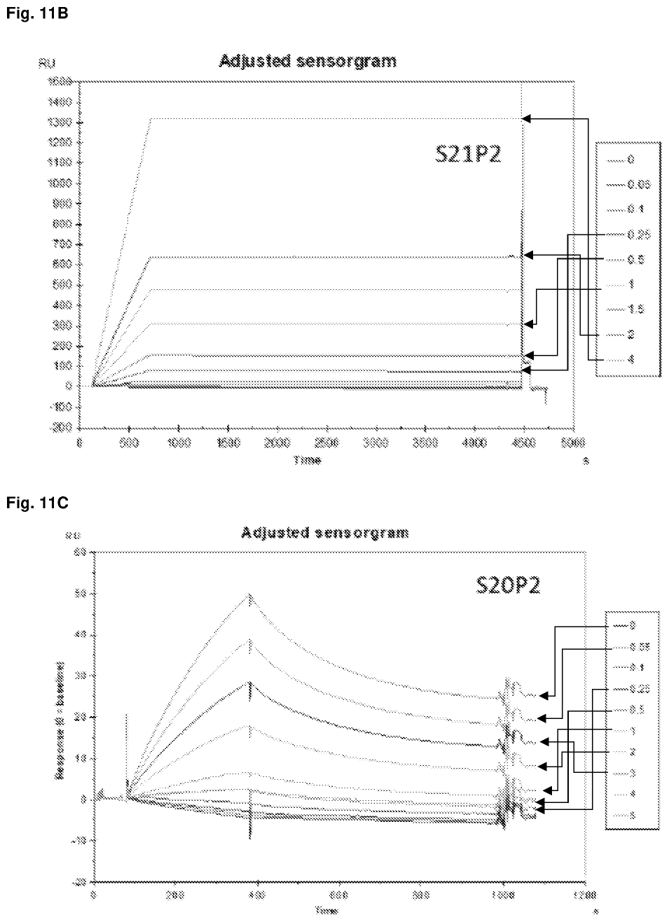

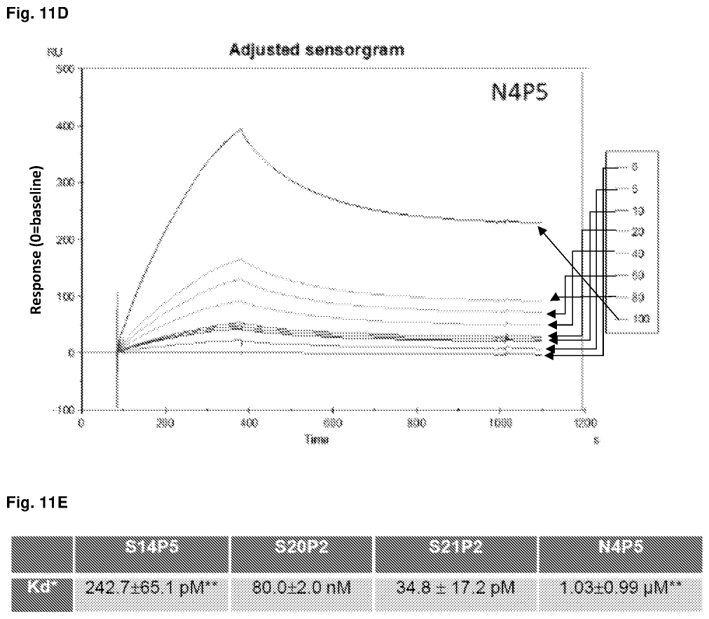

The binding affinity of the SARS-CoV-2 peptide epitopes was determined using the Biacore T200. To perform this experiment, the biotinylated peptides were immobilised onto a streptavidin treated CM5 chip. The chip was passed through COVID-19 IgGs at a range of different concentrations. Kinetic fitting was used to generate the Kd values. The results are shown in FIG. 11A to E. FIG. 11E in particular shows a summary table of the estimated Kd. values for the AuNPs and Based on the analysis, the binding affinities appear to be in the pM to μM range, in the following order: S14/P5/S21P2 (pM)>S20P2 (nM)>N4P5 (μM).

### Example 10—Further Study of Epitope-Functionalised Gold Nanoparticles for Rapid and Selective Detection of SARS-CoV-2 IgG Antibodies

### Experimental Methods

### Materials

Gold(Ill) chloride trihydrate (HAuCl₄·3H₂O), sodium citrate dihydrate, sodium hydroxide, boric acid, N-(2-hydroxyethyl)piperazine-N-ethanesulfonic acid (HEPES), phosphate-buffered saline (PBS), IgG from normal human serum, and normal human plasma were purchased from Sigma-Aldrich. Streptavidin (from Streptomyces avidini) was purchased from Thermo Fisher Scientific. Biotinylated and FITC-labeled peptide epitopes were purchased from Singapore Advanced Biologics Pte Ltd. PEG-biotin (MW=2000 Da) was purchased from Biochempeg Scientific Inc. (Watertown, MA, USA). SARS-CoV-2 S1, S2, and N IgGs, SARS-CoV S and N IgGs, MERS S and N IgGs, and HCoV-HKU1 S and N IgGs were purchased from Sino Biological (Wayne, PA, USA). All reagents were used as received without further purification.

### Characterization of Epitopes Using SPR Spectroscopy

SPR studies were performed using Biacore T200 (Cytiva). Streptavidin, at concentrations of 100 and 5 μg/mL in sodium acetate buffer pH 5 for kinetics and steady-state affinity experiments, respectively, was immobilized on a CM5 chip through EDC/NHS coupling at a flow rate of 10 μL/min. Biotinylated peptide epitopes were then immobilized on the SA-functionalized CM5 chip. Specifically, peptides were diluted in HEPES buffer saline (10 mM HEPES, 150 mM NaCl, 3.4 mM EDTA, 0.05% polysorbate 20, pH 7.4) and subsequently injected to the SA-CM5 surface at a concentration ranging from 10 to 100 μg/mL with a flow rate of 10 μL/min. One flow cell was injected with buffer instead of peptides to serve as a reference flow cell. IgG diluted in HEPES buffered saline (HBS) at concentrations ranging from 0.1 to 80 μg/mL (for kinetics experiments) and from 6.25 to 3000 nM (for steady-state affinity experiments) was injected over the immobilized peptide surface and reference surface at a flow rate of 30 μL/min (for kinetics experiments) and 10 μL/min (for steady-state affinity experiments) at a temperature of 25° C. The complex was allowed to associate (300 s for kinetics experiments and 600 s for steady-state affinity experiments) and dissociate for 600 s. At the end of each cycle of IgG binding, the surfaces were regenerated with a 120 s injection of glycine HCl (pH 1.5). One sample was injected in duplicate over both surfaces to check the quality of the peptide-functionalized surface. Several buffer blanks were also injected intermittently over the course of an experiment for double referencing. Data processing and analysis were performed using Biacore T200 Evaluation Software version 2.0. A double-referencing method was performed to process all data sets. Data from the sample flow cell were referenced first by subtracting data from the reference flow cell to correct for bulk refractive index changes, nonspecific binding, injection noise, matrix effects, and baseline drift. Reference-subtracted data were double-referenced with a blank injection of running buffer to account for any systematic drift over the course of the injection. Double-referenced data were fit to a 1:1 Langmuir binding model for kinetic analysis or steady-state affinity model. Reported values are the mean and standard deviation of at least three independent experiments.

### Characterization of Epitopes Using FP Technique

Black 384-well polystyrene plates were used for FP characterization. In each reaction well, FITC-labeled epitope and the respective SARS-CoV-2 IgG were added with final concentrations of 100 and 200 nM, respectively, in 1×PBS with a total volume of 50 μL. The mixture was then kept in the dark for 15 min at room temperature. FP readings were obtained using a microplate reader (Synergy 2; BioTek) with an excitation of 485 nm and emission of 528 nm. FP values of FITC-labeled epitopes alone (without IgG) were used as a baseline to evaluate the extent of FP increase upon IgG binding, using equation 1 below:

$$\% FP Increase = \frac{\begin{matrix}FP value of epitope with IgG - \\ FP value of epitope without IgG\end{matrix}}{FP value of epitope without IgG} \times 100 \% \qquad ( 1 )$$

### Preparation of Epitope-Functionalized AuNPs

Citrate-capped AuNPs of approximately 13 nm diameter were synthesized by the citrate reduction method as described previously. (59) Briefly, an aqueous solution of HAuCl₄ (1 mM, 50 mL) was brought to reflux while stirring, and then a solution of sodium citrate (38.8 mM, 5 mL) was added quickly. After the solution color changed from pale yellow to deep red, the solution was refluxed for another 20 min and allowed to cool to room temperature. The resulting solution of citrate-AuNPs was stored at 4° C. overnight before further use.

Prior to functionalization with SA and biotinylated epitopes, the citrate buffer of as-prepared AuNPs was exchanged to a borate buffer (pH 8.0, 100 mM, 0.05% Tween) by centrifugation. Then, 10 μL of SA (1 mg/mL in pH 8.0 borate buffer) was added to 500 μL of AuNP solution. The mixture was vortexed briefly and stirred at room temperature for 2 h. Excess SA was removed by centrifugation at 12500 g for 15 min. The pellet was resuspended in borate buffer, and a solution of biotinylated peptide epitope (20 μL, 20 μM) was added to 500 μL of SA-AuNPs. In the case of PEGylated or binary epitope-capped AuNPs, equal volumes of PEG-biotin or biotinylated epitopes (20 μM) were introduced such that the total volume of biotinylated molecules was 20 μL. The mixture was stirred at room temperature for 30 min before centrifugation at 12500 g for 15 min to remove excess biotinylated molecules. The final conjugates were then resuspended in HEPES buffer (pH 7.5, 5 mM) to prepare epitope-functionalized AuNPs for further use. The hydrodynamic diameter and zeta potential of AuNPs at different preparation stages were characterized with Zetasizer Nano ZS (Malvern).

Immunoassay Procedure with Epitope-Tagged AuNPs

SARS-CoV-2 IgG antibodies and control antibodies were first diluted in 1×PBS. In a typical experiment, 5 μL of prediluted antibody solution was added to 45 μL of epitope-functionalized AuNPs. The mixture was spun briefly for 20 s at 6000 rpm using a portable microcentrifuge (Profuge 6K) and incubated for another 30 min at room temperature. Absorbance spectra of the supernatant were recorded using a microplate reader (Synergy 2; BioTek). Sensor response was calculated as the relative change in absorbance at 534 nm. For a time-profile study, the absorbance spectra were recorded at 10 min intervals. For a salt-induced aggregation test, solutions of AuNPs were treated with 1×PBS and the absorbance spectra were recorded after a 30 min incubation. For a selectivity screen against other human coronaviruses, the concentration of antibodies was 100 nM. To investigate the sensitivity of AuNPs in human plasma, SARS-CoV-2 IgG rabbit antibodies were spiked into PBS containing various concentrations of human plasma, such that the final plasma concentration after mixing with AuNPs was 0%, 10%, and 20% (v/v).

### Detection of SARS-CoV-2 IgG in Clinical Samples

A total of 23 COVID-19 patients who tested PCR-positive for SARS-CoV-2 in nasopharyngeal swab samples were classified into four groups based on their clinical severity: asymptomatic (no symptoms of COVID-19), mild (no pneumonia on chest radiographs (CXR) at baseline and during hospital admission), moderate (pneumonia on CXR without hypoxia), and severe (pneumonia on CXR with hypoxia [desaturation to 594%]). Whole blood of patients was collected in BD Vacutainer cell preparation tubes (BD Biosciences) and centrifuged at 1700 g for 20 min to obtain plasma fractions. Samples were then treated with 1% Triton X-100 (ThermoFisher Scientific) for 2 h at room temperature for virus inactivation. A 5 μL amount of the undiluted, inactivated plasma samples was then mixed with 45 μL of PEG-passivated epitope-tagged AuNPs for immunoassay analysis as described in the previous section, such that the final plasma concentration was 10% (v/v). The threshold for detection was calculated from the mean nanosensor responses elicited by healthy patient samples+3 standard deviations.

### Results and Discussion

### Biophysical Characterization of Epitopes' Binding Affinity and Specificity

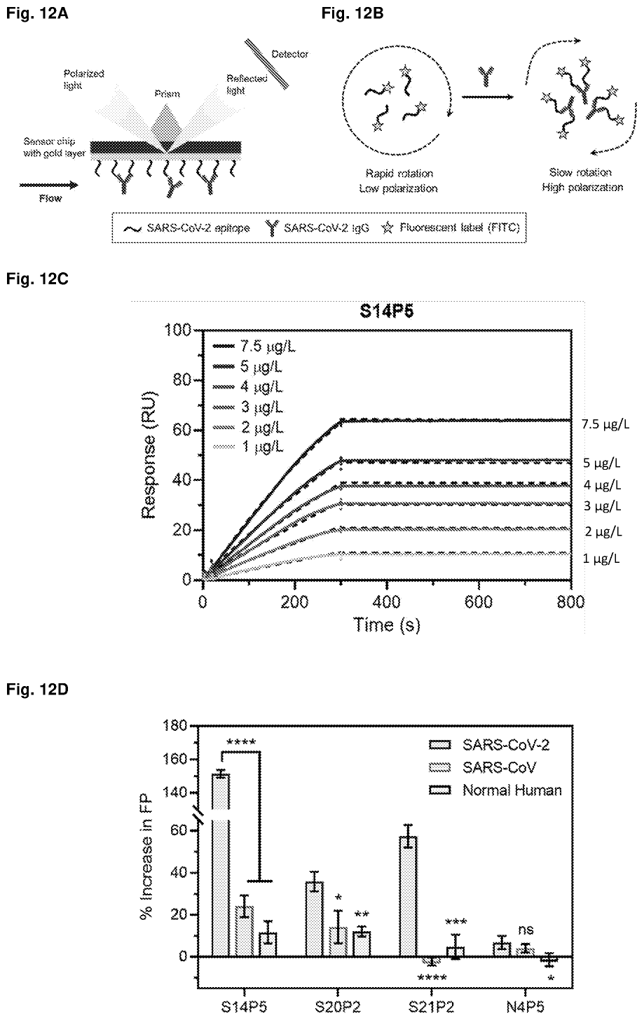

The nanosensor-based platform is inspired by previous discovery of four immunodominant B-cell linear epitopes, namely, S14P5, S20P2, S21P2, and N4P5, which were identified as antigenic targets of IgGs present in the plasma samples of COVID-19 patients. (20) These four epitopes are 18 amino acids in size, and they are located on the S and N viral proteins of SARS-CoV-2 (Table 1). Since the epitopes are located on different viral protein subunits responsible for specific biological functions, it is important to characterize the IgG binding affinity of each peptide. While these epitopes have been screened against plasma samples of COVID-19 patients collected in Singapore using peptide-based ELISA, their binding characteristics toward epitope-specific IgGs of SARS-CoV-2 have not been investigated. Herein, the present inventors first determined the binding affinity and specificity of the four epitopes by using two distinct biophysical characterization tools, specifically SPR spectroscopy and the FP technique (FIGS. 12A and B). SPR spectroscopy detects binding between immobilized ligands and target analytes at the surface of a thin metal film via changes in the refractive index. (40,41) It allows for a label-free, quantitative, and real-time measurement to obtain the binding kinetic constants and affinity. SPR sensorgram data confirmed the binding of all four epitopes when titrated against varying concentrations of epitope-specific SARS-CoV-2 IgGs (FIG. 12c and FIG. 17A to C). Global fitting of kinetic binding data with a 1:1 Langmuir model yielded equilibrium dissociation constants (K_{d}) of approximately 80 and 218 nM for S20P2 and N4P5 epitopes, respectively (Table 2). However, the K_{d} values of S14P5 and S21P2 could not be exactly determined from this kinetic analysis, as these two epitopes exhibit a very slow dissociation step from their respective IgG targets, resulting in an apparent K_{d} in the low pM range beyond the instrument sensitivity limit. (42,43) As such, steady-state analysis without a dissociation step was performed to obtain steady-state K_{d} values (FIG. 18A to D and Table 2). Both kinetic and steady-state analysis confirmed that among the four peptides, S14P5 and S21P2 epitopes exhibit the highest binding affinity toward their respective target IgGs of SARS-CoV-2, followed by S20P2 and then the N4P5 epitope.

TABLE 1
Location, Peptide Sequences, and Isoelectric Points (pI) of
Immunodominant SARS-CoV-2 Linear B-Cell Epitopes (20)

| | location on | | | | |
|---|---|---|---|---|---|
| | SARS-CoV-2 | | starting | ending | |
| epitope | viral antigen | polypeptide sequence | aa | aa | pI^{a} |
| S14P5 | S1 subunit of S | TESNKKFLPFQQFGR | 553 | 570 | 8.26 |
| | protein | DIA | | | |
| | | (SEQ ID NO: 3) | | | |
| S20P2 | S2 subunit of S | GIAVEQDKNTQEVFA | 769 | 786 | 4.68 |
| | protein | QVK | | | |
| | | (SEQ ID NO: 4) | | | |
| S21P2 | S2 subunit of S | PSKPSKRSFIEDLLFN | 809 | 826 | 9.72 |
| | protein | KV | | | |
| | | (SEQ ID NO: 5) | | | |
| N4P5 | N protein | NNAAIVLQLPQGTTLP | 153 | 170 | 8.75 |
| | | KG | | | |
| | | (SEQ ID NO: 6) | | | |

^{a}lsoelectric points were calculated using ProtParam from the SIB ExPASY Bioinformatic Resources Portal. (39)

TABLE 2
K_{d} Obtained from Real-Time Kinetic and Steady-
State Analysis with SPR Spectroscopy

| | S14P5 | S20P2 | S21P2 | N4P5 |
|---|---|---|---|---|
| K_{d} from kinetic | <1.0 | 80 ± 2 | <1.0 | 218 ± 86 |
| analysis (nM) | | | | |
| K_{d} from steady- | 111 ± 15 | 510 ± 34 | 125 ± 7 | 1606 ± 120 |
| state analysis | | | | |
| (nM) | | | | |

An orthogonal FP approach was also pursued to determine the selectivity of these epitopes and to further confirm their IgG binding affinity. FP detects the binding of a fluorescent ligand (FITC-labeled epitopes, in this case) to a larger molecule (IgG) by measuring the increase in the polarization of fluorescence emission induced by the formation of a larger complex. (44) Compared to SPR spectroscopy, FP is a free solution technique that allows for high-throughput screening and does not require any separation of bound and free ligand. (45) The three S-protein-based epitopes (S14P5, S20P2, and S21P2) exhibited a significant increase in FP values upon the introduction of SARS-CoV-2 IgGs relative to the addition of the closely related SARS-CoV IgGs or the control normal human IgG (FIG. 12D). S14P5 exhibited the highest relative FP increase, followed by S21P2 and S20P2. Such a trend indicates specific binding between SARS-CoV-2 IgGs and the selected epitopes, with an affinity order that correlates well with that determined by the SPR spectroscopy. The N4P5 epitope showed a comparably lower percentage of FP increase against all IgGs, indicative of its low binding affinity and specificity. Its lowest response among the four epitopes corroborated earlier findings obtained from SPR measurements. Overall, biophysical characterization using both SPR spectroscopy and FP techniques confirmed that S14P5 and S21P2 epitopes are highly immunogenic, as previously reported. (21) The finding that N4P5 displayed the lowest binding affinity among the four putative epitopes was unexpected, as it showed the highest sensitivity in recognizing IgGs present in the plasma samples of SARS-CoV-2 patients. (20) This discrepancy might be due to the high titer of anti-N IgG in the plasma samples of SARS-CoV-2 patients tested or that the N4P5 epitope recognized only a small proportion of the polyclonal SARS-CoV-2 N rabbit antibodies employed in this study.

### Preparation and Characterization of Epitope-Functionalized AuNPs

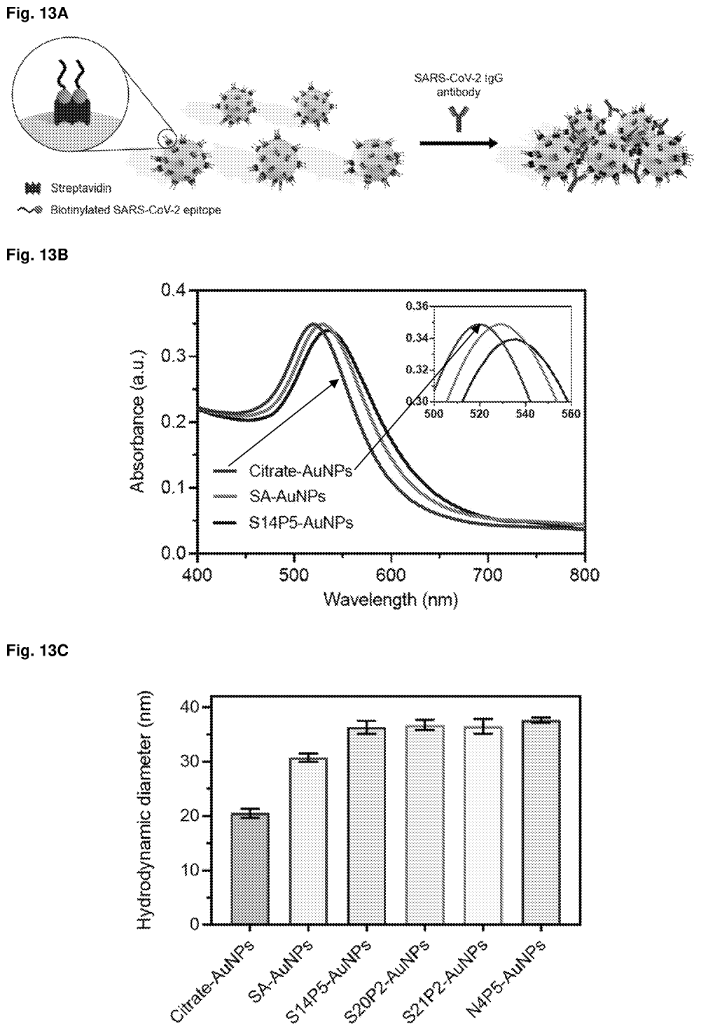

The present inventors next sought to investigate if these epitopes could serve as recognition moieties for SARS-CoV-2-specific IgGs when conjugated to nanoparticles and if the epitope-IgG binding can trigger nanoparticle aggregation to alter AuNPs' plasmonic characteristics (FIG. 13A). As shown in Table 1, the four epitopes have different charges at psychological pH, with S20P2 peptide bearing a negative charge and the other peptides being positively charged. Previous studies have shown that directly conjugating positively charged peptides to negatively charged citrate-capped AuNPs often jeopardizes the colloidal stability of nanoparticles due to electrostatic attractions and multipoint surface binding, limiting the efficacy and versatility of AuNP functionalization with peptides. (46,47) It is thus desirable to construct a more universal platform for AuNP functionalization with epitopes of different charges. Streptavidin (SA) has been demonstrated to provide excellent protection of AuNPs and is known for its high affinity toward biotin via tetrameric binding sites, rendering its ideal use as both a stabilizer corona and a building block for conjugation of biotinylated biomolecules. (48) Taking advantage of the stabilizing property and connectivity of SA, the inventors constructed a multilayered approach of conjugating biotinylated SARS-CoV-2 epitopes to SA-coated AuNPs (FIG. 13A). Considering that the surface area of 13 nm AuNPs is about 547 nm² per nanoparticle and the projected SA surface area is approximately 25 nm², the present inventors estimate that a maximum of 22 SA molecules can be adsorbed per nanoparticle. (49) The concentration of SA was calculated to be above this close-packing threshold to ensure full surface coverage and minimize protein denaturation on the nanoparticle surface (see Methods above). (50) Each biotinylated peptide epitope was then attached to the surface of AuNPs via the high-affinity SA-biotin binding, resulting in single-peptide-functionalized AuNPs (hereafter denoted as S14P5-, S20P2-, S21P2-, and N4P5-AuNPs). Successful conjugation of SA and peptide epitopes to AuNPs was validated by the red-shift in the LSPR peak of AuNPs, from 520 nm in bare AuNPs to 530 and 534 nm after functionalization with SA and peptides, respectively (FIG. 13B). The average hydrodynamic diameters of bare AuNPs and SA-AuNPs were 20.3±1.8 and 31.1±1.5 nm, respectively, indicating the formation of a 5.4 nm thick adsorbed monolayer corresponding to the approximate diameter of SA (˜5 nm) (FIG. 13C). This hydrodynamic diameter further increased by approximately 5 nm upon the attachment of each of the SARS-CoV-2 peptide epitopes. The zeta potential of epitope-tagged AuNPs varies with the net charge of the epitopes as governed by their respective isoelectric point (pl) (FIG. 19). Additionally, SA- and epitope-functionalized AuNPs demonstrated higher colloidal stability in high ionic strength solution than bare AuNPs (FIG. 20A to F), further validating the successful functionalization of SARS-CoV-2 epitopes on the AuNP surface.

Sars-CoV-2 IgG Detection with Epitope-Tagged AuNPs

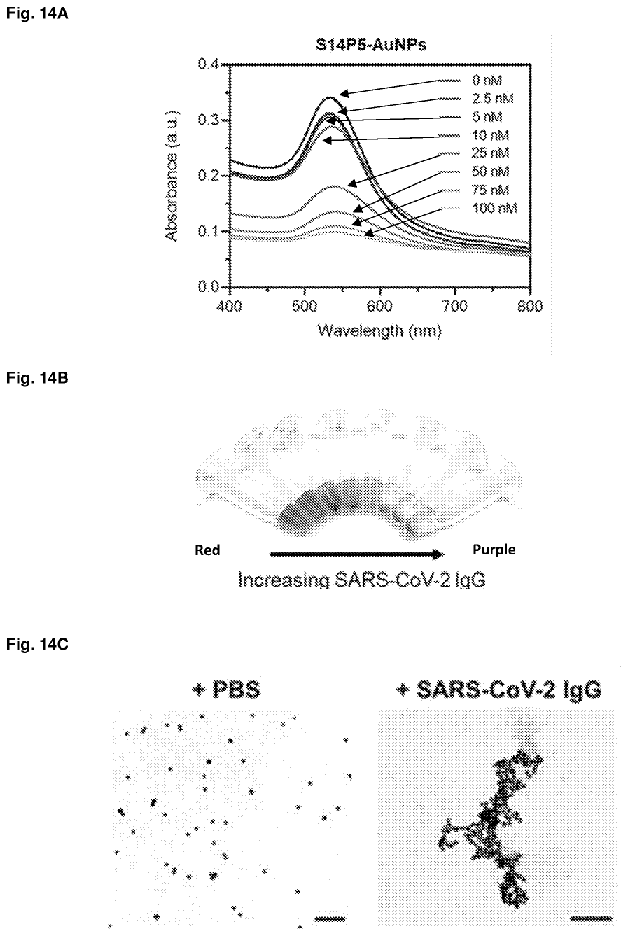

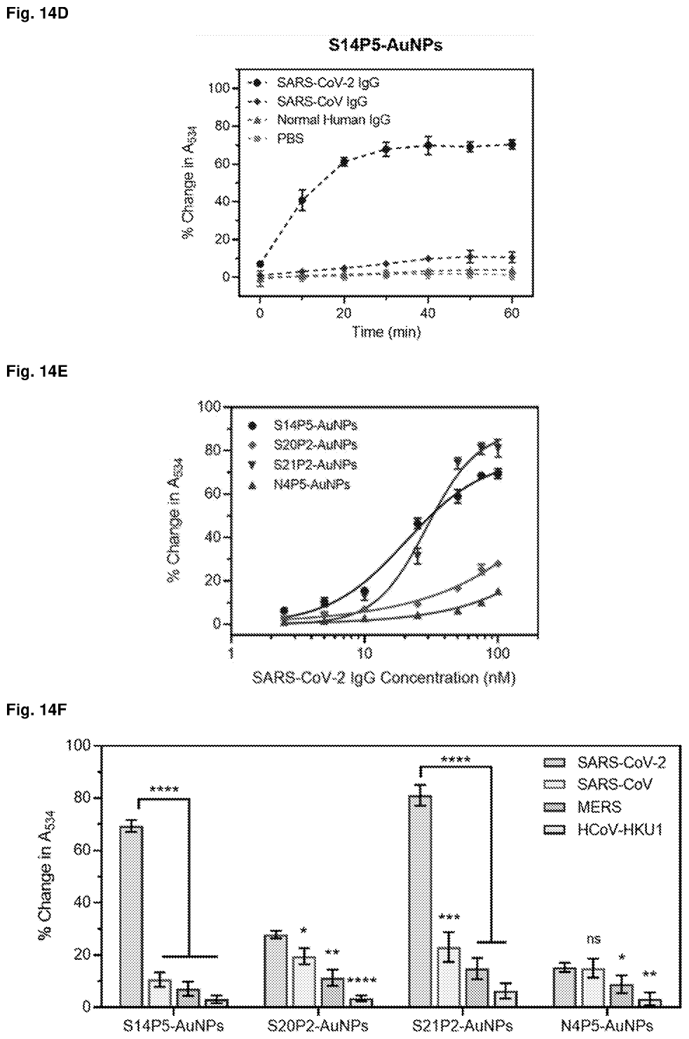

The inventors next analyzed the change in the plasmonic characteristics of epitope-functionalized AuNPs upon mixing with target SARS-CoV-2 IgGs. S14P5-AuNPs were chosen for initial studies, as the S14P5 epitope exhibited the highest binding affinity among the four selected epitopes as characterized by SPR and FP techniques. This epitope is also located in close proximity to the receptor-binding domain (RBD) and showed high recognition toward SARS-CoV-2 patients with 97% sensitivity using peptide-based ELISA. (20,21) The introduction of anti-SARS-CoV-2 S1 IgG elicited a dose-dependent aggregation profile of S14P5-AuNPs, resulting in a decrease in the plasmon peak intensity (FIG. 14A). Such a response was absent when the nanoparticles were treated with normal human IgG and SARS-CoV anti-S IgG (FIG. 21). The anti-SARS-CoV-2 S1 IgG-induced nanoparticle aggregation could be conveniently observed by the naked eye (FIG. 14B). From the absorbance spectrum, the inventors note here that there is only a minor shift of the plasmon wavelength due to SARS-CoV-2 IgG treatment, indicating limited interparticle plasmonic coupling presumably because the interparticle distance between S14P5-AuNPs upon IgG binding is larger than the gold core diameter. (51,52) Nonetheless, the specific antibody-induced aggregation of S14P5-AuNPs was corroborated by transmission electron microscopy (TEM) images, which showed the clustering of nanoparticles in the presence of their target IgG (FIG. 14C). A steric-kinetic driving force, arising from spatial competition of IgGs for a limited number of epitopes on the AuNP surface, has been proposed as the mechanism underlying antibody-triggered aggregation of epitope-tagged AuNPs. (53) The response and kinetics of S14P5-AuNPs aggregation were monitored by analyzing the comparative decrease in peak absorbance at 534 nm at 10 min intervals for 60 min. When challenged with anti-SARS-CoV-2 S1 IgG, the normalized plasmon peak intensity of S14P5-AuNPs decreased by 40.2% in the first 10 min, leveling off to 67.3% within 30 min (FIG. 14D). Despite the high level of homology between the S protein of SARS-CoV-2 and SARS-CoV (76%), the normalized sensor response of S14P5-AuNPs was 6-fold lower (11.4%) when challenged with SARS-CoV IgG after 60 min. Taken together, these results highlight the rapid response and high selectivity of the S14P5 epitope-functionalized AuNPs in discriminating SARS-CoV-2 IgG from other IgGs.

The IgG dose-dependent responses of the other epitope-functionalized AuNPs were analyzed to characterize their sensitivity. Although all four epitopes had been shown to display >95% sensitivity in detecting SARS-CoV-2 patients 23 days postillness onset (pio) with peptide-based ELISA, (20) the inventors found that epitope-functionalized AuNPs exhibited vastly different levels of sensitivity in recognizing SARS-CoV-2-specific IgGs (FIG. 14E and FIG. 22A to C). In the order of their sensitivity, S14P5-AuNPs and S21P2-AuNPs showed a lower limit of detection (LOD) of 4.1 and 5.1 nM, respectively, while the LODs of S20P2-AuNPs and N4P5-AuNPs were higher, at 15.4 and 25.8 nM, respectively (see calculation in Methods above). These figures of merit suggest that epitope-functionalized AuNPs can be potentially used to detect IgG in convalescent COVID-19 patient samples, in which IgG has been estimated to reach approximately 16.5 μg/mL (˜110 nM) at 21-25 days pio and remain at 11.4 μg/mL (˜76 nM) until 41 days pio. (54) Additionally, the sensitivity trend between the four epitope-tagged AuNPs aligned with the binding affinity trend obtained from SPR spectroscopy characterization, confirming that the optical response of epitope-functionalized AuNPs arose from the IgG-epitope binding interaction.

The inventors next fully assessed the selectivity of all four epitope-functionalized AuNPs toward epitope-specific IgGs of the closely related SARS-CoV as well as two other human betacoronaviruses, namely, MERS and HCoV-HKU1 (FIG. 14F). AuNPs functionalized with epitopes of the S1 and S2 protein subunits (S14P5-, S20P2-, and S21P2-AuNPs) showed minimal sensor response in the presence of MERS and HCoV-HKU1 IgGs. The minimal cross-reactivity may be due to the fact that these coronaviruses are only distantly related to SARS-CoV-2 at the protein level. (55) For the three S epitopes, the closely related SARS-CoV IgG elicited slightly higher level of attenuation in the nanoparticles' absorbance peak intensity compared to those elicited by the distantly related MERS and HCoV-HKU1 IgGs, but they are still substantially lower than the responses elicited by SARS-CoV-2 IgG. Among the four epitope-tagged AuNPs studied here, N4P5-AuNPs exhibited the lowest affinity and the highest cross-reactivity with IgGs of other human coronaviruses, with a similar degree of sensor response toward SARS-CoV-2 (15.4%) and SARS-CoV IgGs (14.9%). This cross-reactivity is not surprising, as the N protein of SARS-CoV-2 shares ˜90% homology in amino acid sequences with that of SARS-CoV. The N4P5 peptide itself also shares 94% sequence identity with the corresponding hit region on SARS-CoV N protein, the highest homology among the four epitopes studied here. (20) Nonetheless, for the other three epitopes, the highest sensor response for all peptide-AuNPs was elicited by SARS-CoV-2 IgGs, validating their choice as highly specific nanoparticle recognition moieties for the development of targeted SARS-CoV-2 serological assays.

### Conjugation of Dual Epitopes and Passivation Strategy

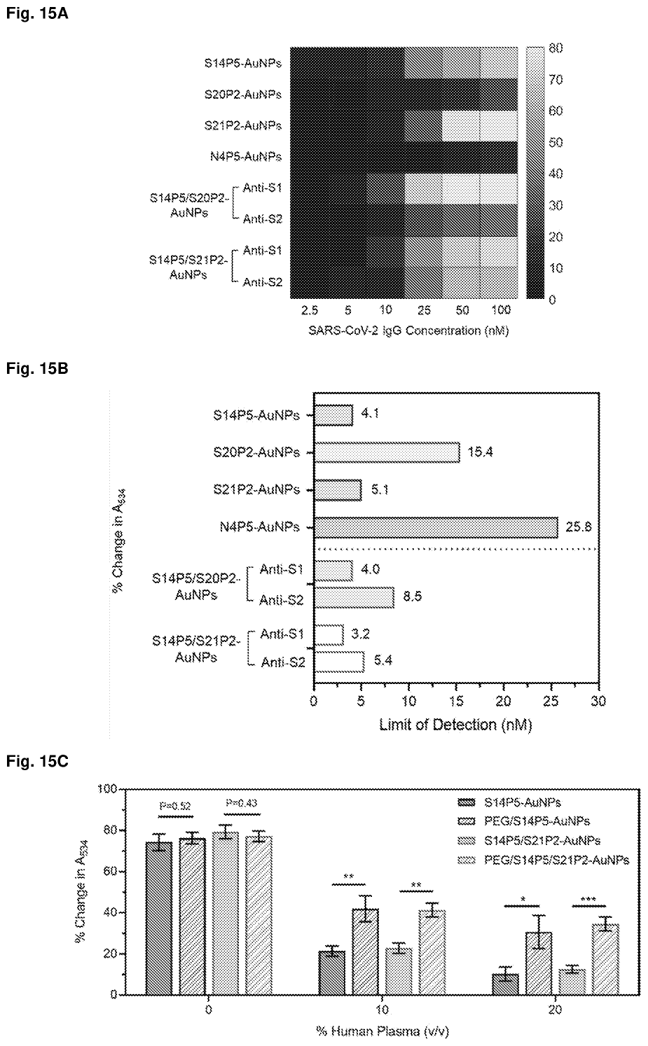

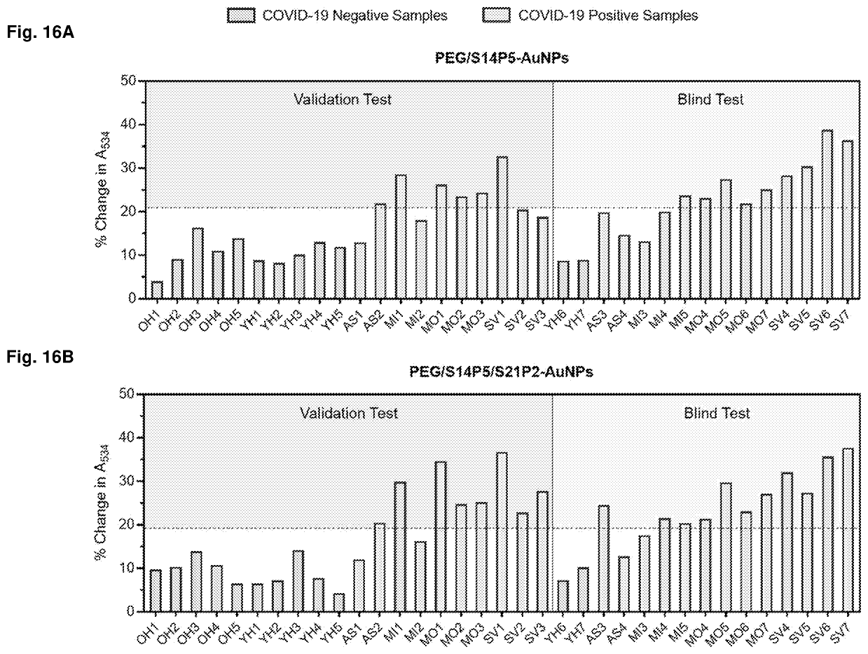

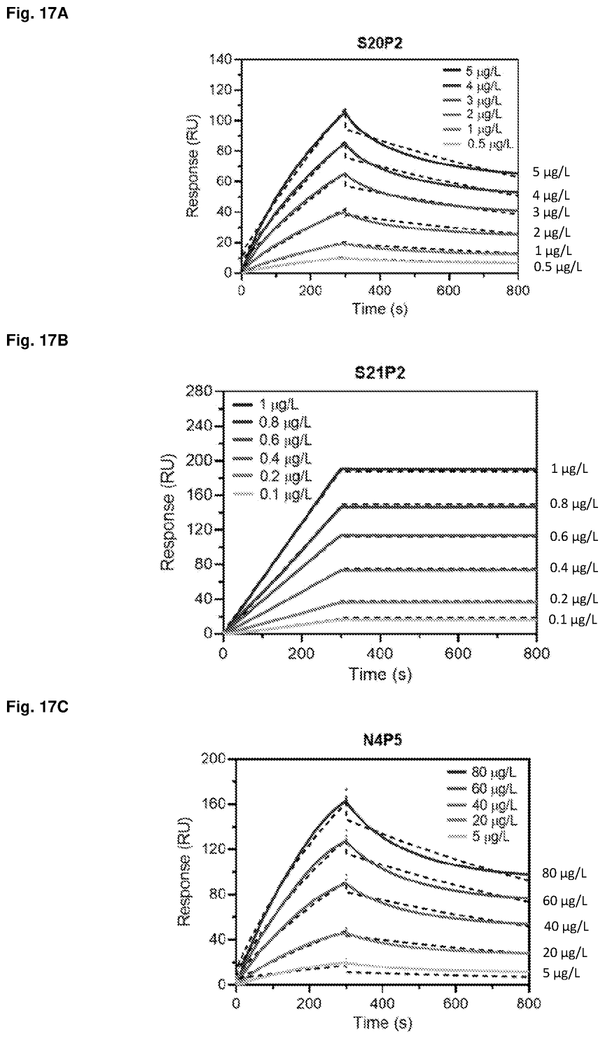

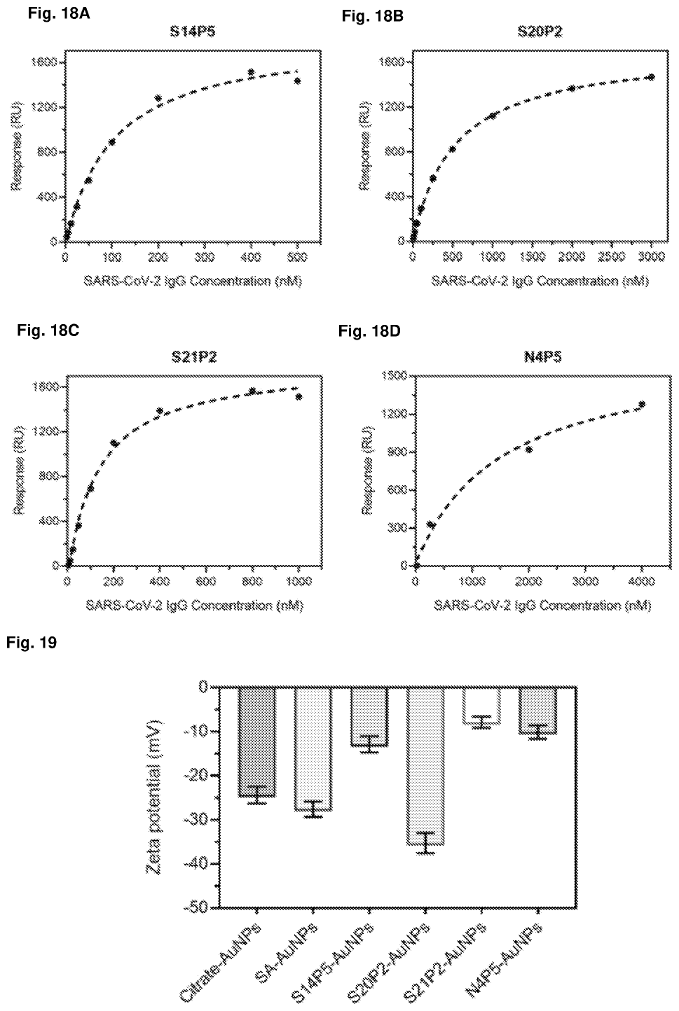

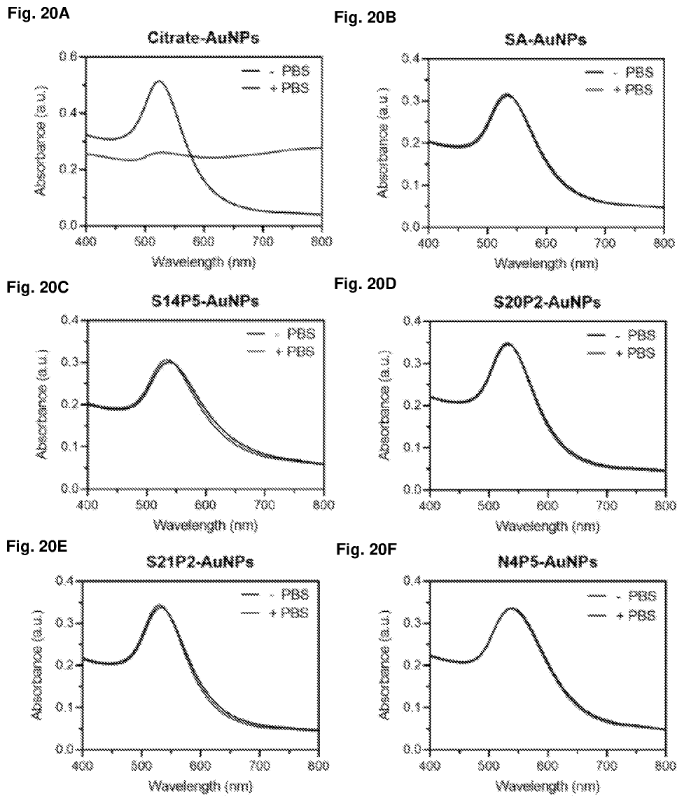

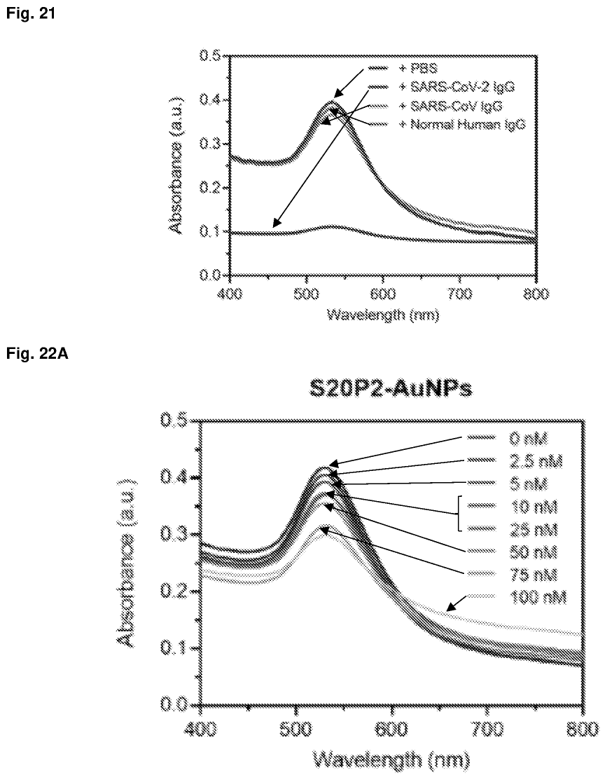

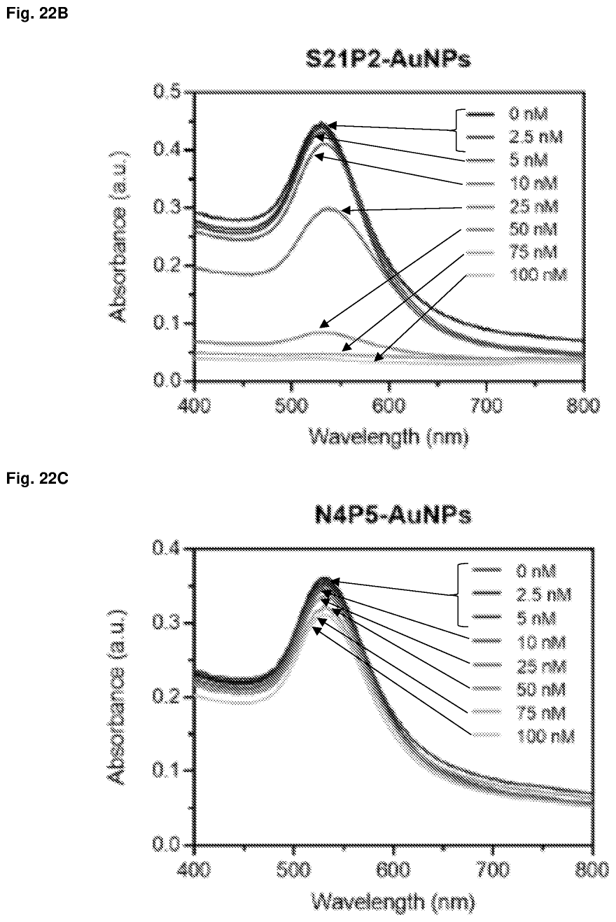

While the recognition affinity of each epitope-tagged AuNP toward SARS-CoV-2 IgG has been demonstrated, the inventors explored strategies to further improve the sensitivity and versatility of the nanosensor platform by co-immobilizing two different epitopes on the nanoparticle surface to form dual epitope-tagged AuNPs. The ability to conveniently attach multiple epitopes through facile nanoparticle functionalization for potentially improving assay performance represents a distinct advantage of epitope-nanoparticle platforms over conventional serological assays, most of which are based on single SARS-CoV-2 recombinant proteins. (14) As N4P5-AuNPs were found to be the least sensitive in detecting anti-SARS-CoV-2 IgG, we focused on the combinatorial use of S14P5, S20P2, and S21P2 epitopes. In particular, binary combinations of S14P5 with S20P2 or S21P2 epitopes were explored, as these pairs (S14P5/S20P2 and S14P5/S21P2) each contain epitopes located on both S1 and S2 protein subunits, potentially extending their capability to detect both anti-S1 and anti-S2 IgGs of SARS-CoV-2. As hypothesized, the dual-peptide-functionalized S14P5/S20P2-AuNPs and S14P5/S21P2-AuNPs were able to recognize both anti-S1 and anti-S2 IgGs of SARS-CoV-2, in contrast to the single-peptide-AuNPs, which could only recognize either anti-S1 or anti-S2 IgG (FIG. 23). The sensitivity of single- and dual-peptide AuNPs toward varying IgG concentrations was then juxtaposed through a heat map, in which a higher sensor response translates to a brighter color profile (FIG. 15A). Interestingly, both S14P5/S20P2-AuNPs and S14P5/S21P2-AuNPs could detect anti-SARS-CoV-2 S1 IgG with higher sensitivity (LODs of 4.0 and 3.2 nM, respectively) than AuNPs conjugated with S14P5 alone (LOD of 4.1 nM). Comparing between the two dual-epitope-functionalized AuNPs, the LODs of S14P5/S21P2-AuNPs are lower than those of S14P5/S20P2-AuNPs for the detection of anti-S1 and anti-S2 IgGs of SARS-CoV-2 (FIG. 15B).

To further optimize the nanosensor design for clinical sample detection, we evaluated the compatibility of epitope-functionalized AuNPs in relevant biofluids by monitoring the sensor response upon exposure to SARS-CoV-2 IgG in different concentrations of human plasma. Both S14P5-AuNPs and dual-peptide-functionalized S14P5/S21P2-AuNPs were chosen as the model nanoparticles for this investigation due to their high sensitivity. In the case of S14P5/S21P2-AuNPs, nanosensor response against 100 nM anti-S1 IgG was attenuated with increasing concentrations of human plasma, from 78.2% in pure PBS to 22.8% and 14.6% in 10-fold and 5-fold diluted human plasma, respectively (FIG. 15C). S14P5-AuNPs showed a similar trend in the modulation of sensor response with increasing plasma concentration (FIG. 15C). The diminished sensor response in high plasma background may be caused by nonspecific adsorption of plasma proteins to the nanosensor constructs, which subsequently hindered the binding of IgG to the epitopes on the AuNPs. To reduce the inhibitory effects of biofouling on nanosensor response, we passivated the surface of AuNPs with polyethylene glycol (PEG) moieties. The hydrodynamic diameters of PEGylated S14P5-AuNPs and S14P5/S21P2-AuNPs were similar to the nonpassivated counterparts (FIG. 24). The zeta potentials of these PEG-passivated AuNPs were less negative than the nonpassivated nanoparticles (FIG. 25). These trends indicate the successful conjugation of the neutrally charged PEG molecule, along with the epitopes, on the surface of SA-AuNPs. As biotinylated PEG and epitopes have similar molecular weights and their conjugation affinity toward SA-AuNPs is mainly governed by the streptavidin-biotin binding, the inventors assumed a uniform distribution between PEG and epitopes on the nanoparticle surface. The PEG-passivated AuNPs recognized SARS-CoV-2 IgG with similar sensitivity to non-PEGylated AuNPs in PBS (FIG. 15C). Importantly, passivated nanosensors demonstrated enhanced sensitivity compared with the nonpassivated nanoparticles to SARS-CoV-2 IgG in human serum, with 46.5% and 35.2% sensor response in 10-fold and 5-fold diluted serum, respectively, in the case of PEGylated S14P5/S21P2-AuNPs. These results suggest that PEG passivation of epitope-functionalized AuNPs partially mitigated nanosensor biofouling, strengthening the practical utility of epitope-functionalized nanosensors as in vitro diagnostic tools for SARS-CoV-2 IgG detection in plasma.

### Sensitive and Selective SARS-CoV-2 IgG Detection in Clinical Samples

Finally, the inventors utilized the PEG-passivated S14P5-AuNPs and S14P5/S21P2-AuNPs to detect SARS-CoV-2 IgG in clinical patient samples. In the first trial, a cohort of 20 clinical samples from Singapore, consisting of 10 PCR-confirmed COVID-19 samples of different severity at median 34.5 days pio and 10 healthy patient samples, was used to validate the assay utility. Plasma samples were inactivated with 1% (v/v) Triton X-100 prior to incubation with epitope-tagged AuNPs for 1 h. The final plasma concentration after mixing with AuNPs was 10% (v/v). From the 10 COVID-19-positive samples, the mono-epitope S14P5-AuNPs and dual-epitope S14P5/S21P2-AuNPs accurately detected 6 and 8 samples as positive, respectively (FIGS. 16A and B). The higher sensitivity of the dual-epitope S14P5/S21P2-AuNPs could be attributed to the presence of both the S14P5 and S21P2 epitopes on the AuNP surface, enabling recognition of a wider range of SARS-CoV-2-specific IgGs (anti-S1 and anti-S2) present in COVID-19 plasma samples. This observation is consistent with earlier experiments using purified polyclonal SARS-CoV-2 antibodies (FIGS. 15A and B). The 2 false negative samples which could not be detected with S14P5/S21P2-AuNPs were asymptomatic and mild cases, which are known to have decreased IgG levels. (56,57) There was no false positive result from both types of nanoparticles. Encouraged by these preliminary results, the inventors conducted a blind test using a cohort of 15 samples, of which 13 samples were PCR-confirmed COVID-19 samples of different severity (median 37 days pio) and 2 samples were obtained from healthy patients prior to the COVID-19 outbreak. For both mono- and dual-epitope nanosensors, the COVID-19-negative samples elicited a sensor response that was much lower than the previously defined cutoff (FIGS. 16A and B). As expected, the dual S14P5/S21P2-AuNPs showed a higher sensitivity than the mono S14P5-AuNPs, with successful detection of 11 and 9 positive cases from 13 COVID-19-positive samples by the former and the latter, respectively. Similar to the validation test, the two false negatives in the case of S14P5/S21P2-AuNPs were asymptomatic and mild cases. Overall, the sensitivity of S14P5-AuNPs and S14P5/S21P2-AuNPs was 65% and 83%, respectively, with 100% specificity for both nanoparticles when tested against 35 clinical patient samples. The high specificity and sensitivity of PEG-passivated S14P5/S21P2-AuNPs in clinical sample detection demonstrates the potential of this nanosensor platform to serve as promising alternatives to classical immunoassays in COVID-19 serodiagnostics.

### CONCLUSIONS

In summary, the inventors have developed a nanosensor platform for rapid detection of SARS-CoV-2 antibodies by exploiting specific epitope-IgG interactions. This assay harnesses the innate ability of specific linear B-cell epitopes to selectively recognize SARS-CoV-2 IgG, together with the distance-dependent plasmonic properties of AuNPs. Prior to nanosensor preparation, the inventors determined the binding affinity and specificity of four immunodominant linear B-cell epitopes toward SARS-CoV-2 IgG with SPR and FP characterization tools. A generic SA-mediated conjugation of the epitopes to AuNPs was developed to produce nanosensors that can recognize cognate SARS-CoV-2 IgG antibodies, detectable as changes in the absorption spectra of AuNPs. The epitope-functionalized AuNPs showed nM-range LODs in recognizing SARS-CoV-2 IgG, with S14P5-AuNPs and S21P2 AuNPs being the most sensitive and N4P5-AuNPs being the least sensitive nanoparticles, which is in accordance with the binding affinity trend as determined by SPR and FP techniques in this study. Compared to single-epitope-functionalized AuNPs, the attachment of two pM-affinity epitopes (S14P5 and S21P2) on the AuNP surface further enhanced the sensitivity of the assay to the low nM range and expanded the detection capability to recognize SARS-CoV-2 IgGs targeting different regions of the viral antigen. Furthermore, passivation of the nanosensor surface with PEG enhanced the assay sensitivity in human plasma. Notably, PEGylated S14P5/S21P2-functionalized AuNPs could detect SARS-CoV-2 IgGs in the plasma samples from COVID-19 convalescent patients and discriminate between COVID-19-positive and -negative samples. As these four epitopes are conserved within the circulating COVID-19 variants, the proposed platform holds great potential to serve as highly specific and cost-effective serodiagnostics to control the current pandemic. The nanosensor design described here is also amenable to facile conjugation of different biotinylated epitopes or biomolecules to combat new mutations or future infectious disease outbreaks. More detailed studies on the effect of nanoparticle size, core metal compositions, and signal amplification strategies can be explored, offering further control on the dynamic range and sensitivity to tune analytical performance. Optimization of this nanosensor platform for IgG detection in other biofluids, such as saliva samples, can also be pursued to enable noninvasive and easy-to-scale serological testing at the point of care. (58) Taken together, the results presented in this work highlight the potential utility of epitope-functionalized AuNPs in vaccine efficiency assessments, seroprevalence surveys, and targeted immunoassays to identify individuals with both late-stage and past SARS-CoV-2 infections, expanding the current tool box available for SARS-CoV-2 serodiagnosis.

### REFERENCES

1. Bhalla, N.; Pan, Y.; Yang, Z.; Payam, A. F. Opportunities and Challenges for Biosensors and Nanoscale Analytical Tools for Pandemics: COVID-19. ACS Nano 2020, 14, 7783-7807, DOI: 10.1021/acsnano.0c04421
2. Long, Q.-X.; Liu, B.-Z.; Deng, H.-J.; Wu, G.-C.; Deng, K.; Chen, Y.-K.; Liao, P.; Qiu, J.-F.; Lin, Y.; Cai, X.-F.; Wang, D.-Q.; Hu, Y.; Ren, J.-H.; Tang, N.; Xu, Y.-Y.; Yu, L.-H.; Mo, Z.; Gong, F.; Zhang, X.-L.; Tian, W.-G. Antibody Responses to SARS-CoV-2 in Patients with COVID-19. Nat. Med. 2020, 26, 845-848, DOI: 10.1038/s41591-020-0897-1
3. Liu, X.; Wang, J.; Xu, X.; Liao, G.; Chen, Y.; Hu, C.-H. Patterns of IgG and IgM Antibody Response in COVID-19 Patients. Emerging Microbes Infect. 2020, 9, 1269-1274, DOI: 10.1080/22221751.2020.1773324
4. Winter, A. K.; Hegde, S. T. The Important Role of Serology for COVID-19 Control. Lancet Infect. Dis. 2020, 20, 758-759, DOI: 10.1016/S1473-3099(20)30322-4
5. Petherick, A. Developing Antibody Tests for SARS-CoV-2. Lancet 2020, 395,1101-1102, DOI: 10.1016/S0140-6736(20)30788-1
6. Grossberg, A. N.; Koza, L. A.; Ledreux, A.; Prusmack, C.; Krishnamurthy, H. K.; Jayaraman, V.; Granholm, A.-C.; Linseman, D. A. A Multiplex Chemiluminescent Immunoassay for Serological Profiling of COVID-19-Positive Symptomatic and Asymptomatic Patients. Nat. Commun. 2021, 12, 740, DOI: 10.1038/s41467-021-21040-7
7. La Marca, A.; Capuzzo, M.; Paglia, T.; Roli, L.; Trenti, T.; Nelson, S. M. Testing for SARS-CoV-2 (COVID-19): A Systematic Review and Clinical Guide to Molecular and Serological In-Vitro Diagnostic Assays. Reprod. BioMed. Online 2020, 41, 483-499, DOI: 10.1016/j.rbmo.2020.06.001
8. Zou, L.; Ruan, F.; Huang, M.; Liang, L.; Huang, H.; Hong, Z.; Yu, J.; Kang, M.; Song, Y.; Xia, J.; Guo, Q.; Song, T.; He, J.; Yen, H.-L.; Peiris, M.; Wu, J. SARS-CoV-2 Viral Load in Upper Respiratory Specimens of Infected Patients. N. Engl. J. Med. 2020, 382,1177-1179, DOI: 10.1056/NEJMc2001737
9. To, K. K.-W.; Tsang, O. T.-Y.; Leung, W.-S.; Tam, A. R.; Wu, T.-C.; Lung, D. C.; Yip, C. C.-Y.; Cai, J.-P.; Chan, J. M.-C.; Chik, T. S.-H.; Lau, D. P.-L.; Choi, C. Y.-C.; Chen, L.-L.; Chan, W.-M.; Chan, K.-H.; Ip, J. D.; Ng, A. C.-K.; Poon, R. W.-S.; Luo, C.-T.; Cheng, V. C.-C. Temporal Profiles of Viral Load in Posterior Oropharyngeal Saliva Samples and Serum Antibody Responses during Infection by SARS-CoV-2: An Observational Cohort Study. Lancet Infect. Dis. 2020, 20, 565-574, DOI: 10.1016/S1473-3099(20)30196-1
10. Earle, K. A.; Ambrosino, D. M.; Fiore-Gartland, A.; Goldblatt, D.; Gilbert, P. B.; Siber, G. R.; Dull, P.; Plotkin, S. A. Evidence for Antibody as a Protective Correlate for COVID-19 Vaccines. Vaccine 2021, DOI: 10.1016/j.vaccine.2021.05.063
11. Jin, P.; Li, J.; Pan, H.; Wu, Y.; Zhu, F. Immunological Surrogate Endpoints of COVID-2019 Vaccines: The Evidence We Have versus the Evidence We Need. Signal Transduction and Targeted Therapy 2021, 6, 48, DOI: 10.1038/s41392-021-00481-y
12. Doria-Rose, N.; Suthar, M. S.; Makowski, M.; O'Connell, S.; Mcdermott, A. B.; Flach, B.; Ledgerwood, J. E.; Mascola, J. R.; Graham, B. S.; Lin, B. C.; O'Dell, S.; Schmidt, S. D.; Widge, A. T.; Edara, V.-V.; Anderson, E. J.; Lai, L.; Floyd, K.; Rouphael, N. G.; Zarnitsyna, V.; Roberts, P. C. Antibody Persistence through 6 Months after the Second Dose of mRNA-1273 Vaccine for COVID-19. N. Engl. J. Med. 2021, 384,2259-2261, DOI: 10.1056/NEJMc2103916
13. Okba, N. M. A.; Müller, M. A.; Li, W.; Wang, C.; GeurtsvanKessel, C. H.; Corman, V. M.; Lamers, M. M.; Sikkema, R. S.; de Bruin, E.; Chandler, F. D.; Yazdanpanah, Y.; Le Hingrat, Q.; Descamps, D.; Houhou-Fidouh, N.; Reusken, C. B. E. M.; Bosch, B.-J.; Drosten, C.; Koopmans, M. P. G.; Haagmans, B. L. Severe Acute Respiratory Syndrome Coronavirus 2-Specific Antibody Responses in Coronavirus Disease Patients. Emerging Infect. Dis. 2020, 26,1478-1488, DOI: 10.3201/eid2607.200841
14. Grzelak, L.; Temmam, S.; Planchais, C.; Demeret, C.; Tondeur, L.; Huon, C.; Guivel-Benhassine, F.; Staropoli, I.; Chazal, M.; Dufloo, J.; Planas, D.; Buchrieser, J.; Rajah, M. M.; Robinot, R.; Porrot, F.; Albert, M.; Chen, K.-Y.; Crescenzo-Chaigne, B.; Donati, F.; Anna, F. A Comparison of Four Serological Assays for Detecting Anti-SARS-CoV-2 Antibodies in Human Serum Samples from Different Populations. Sci. Transl. Med. 2020, 12, eabc3103 DOI: 10.1126/scitranslmed.abc3103
15. Grifoni, A.; Sidney, J.; Zhang, Y.; Scheuermann, R. H.; Peters, B.; Sette, A. A Sequence Homology and Bioinformatic Approach Can Predict Candidate Targets for Immune Responses to SARS-CoV-2. Cell Host Microbe 2020, 27, 671-680, DOI: 10.1016/j.chom.2020.03.002
16. Hicks, J.; Klumpp-Thomas, C.; Kalish, H.; Shunmugavel, A.; Mehalko, J.; Denson, J.-P.; Snead, K. R.; Drew, M.; Corbett, K. S.; Graham, B. S.; Hall, M. D.; Memoli, M. J.; Esposito, D.; Sadtler, K. Serologic Cross-Reactivity of SARS-CoV-2 with Endemic and Seasonal Betacoronaviruses. J. Clin. Immunol. 2021, DOI: 10.1007/si10875-021-00997-6
17. Ayoglu, B.; Schwenk, J. M.; Nilsson, P. Antigen Arrays for Profiling Autoantibody Repertoires. Bioanalysis 2016, 8, 1105-1126, DOI: 10.4155/bio.16.31
18. Andresen, H.; Grotzinger, C. Deciphering the Antibodyome—Peptide Arrays for Serum Antibody Biomarker Diagnostics. Curr. Proteomics 2009, 6, 1-12, DOI: 10.2174/157016409787847457
19. Meng, X.; Wei, J.; Wang, Y.; Zhang, H.; Wang, Z. The Role of Peptide Microarrays in Biomedical Research. Anal. Methods 2018, 10, 4614-4624, DOI: 10.1039/C8AY01442F
20. Amrun, S. N.; Lee, C. Y.-P.; Lee, B.; Fong, S.-W.; Young, B. E.; Chee, R. S.-L.; Yeo, N. K.-W.; Torres-Ruesta, A.; Carissimo, G.; Poh, C. M.; Chang, Z. W.; Tay, M. Z.; Chan, Y.-H.; Chen, M. I.-C.; Low, J. G.-H.; Tambyah, P. A.; Kalimuddin, S.; Pada, S.; Tan, S.-Y.; Sun, L. J. Linear B-Cell Epitopes in the Spike and Nucleocapsid Proteins as Markers of SARS-CoV-2 Exposure and Disease Severity. EBioMedicine 2020, 58, 102911, DOI: 10.1016/j.ebiom.2020.102911
21. Poh, C. M.; Carissimo, G.; Wang, B.; Amrun, S. N.; Lee, C. Y.-P.; Chee, R. S.-L.; Fong, S.-W.; Yeo, N. K.-W.; Lee, W.-H.; Torres-Ruesta, A.; Leo, Y.-S.; Chen, M. I.-C.; Tan, S.-Y.; Chai, L. Y. A.; Kalimuddin, S.; Kheng, S. S. G.; Thien, S.-Y.; Young, B. E.; Lye, D. C.; Hanson, B. J. Two Linear Epitopes on the SARS-CoV-2 Spike Protein that Elicit Neutralising Antibodies in COVID-19 Patients. Nat. Commun. 2020, 11, 2806, DOI: 10.1038/s41467-020-16638-2
22. Carter, L. J.; Garner, L. V.; Smoot, J. W.; Li, Y.; Zhou, Q.; Saveson, C. J.; Sasso, J. M.; Gregg, A. C.; Soares, D. J.; Beskid, T. R.; Jervey, S. R.; Liu, C. Assay Techniques and Test Development for COVID-19 Diagnosis. ACS Cent. Sci. 2020, 6, 591-605, DOI: 10.1021/acscentsci.0c00501
23. Shan, B.; Broza, Y. Y.; Li, W.; Wang, Y.; Wu, S.; Liu, Z.; Wang, J.; Gui, S.; Wang, L.; Zhang, Z.; Liu, W.; Zhou, S.; Jin, W.; Zhang, Q.; Hu, D.; Lin, L.; Zhang, Q.; Li, W.; Wang, J.; Liu, H. Multiplexed Nanomaterial-Based Sensor Array for Detection of COVID-19 in Exhaled Breath. ACS Nano 2020, 14, 12125-12132, DOI: 10.1021/acsnano.0c05657
24. Zhong, J.; Rösch, E. L.; Viereck, T.; Schilling, M.; Ludwig, F. Toward Rapid and Sensitive Detection of SARS-CoV-2 with Functionalized Magnetic Nanoparticles. ACS Sensors 2021, 6, 976-984, DOI: 10.1021/acssensors.0c02160
25. Parihar, A.; Ranjan, P.; Sanghi, S. K.; Srivastava, A. K.; Khan, R. Point-of-Care Biosensor-Based Diagnosis of COVID-19 Holds Promise to Combat Current and Future Pandemics. ACS Applied Bio Materials 2020, 3, 7326-7343, DOI: 10.1021/acsabm.0c01083
26. Kevadiya, B. D.; Machhi, J.; Herskovitz, J.; Oleynikov, M. D.; Blomberg, W. R.; Bajwa, N.; Soni, D.; Das, S.; Hasan, M.; Patel, M.; Senan, A. M.; Gorantla, S.; Mcmillan, J.; Edagwa, B.; Eisenberg, R.; Gurumurthy, C. B.; Reid, S. P. M.; Punyadeera, C.; Chang, L.; Gendelman, H. E. Diagnostics for SARS-CoV-2 Infections. Nat. Mater. 2021, 20, 593-605, DOI: 10.1038/s41563-020-00906-z
27. Moitra, P.; Alafeef, M.; Dighe, K.; Frieman, M. B.; Pan, D. Selective Naked-Eye Detection of SARS-CoV-2 Mediated by N Gene Targeted Antisense Oligonucleotide Capped Plasmonic Nanoparticles. ACS Nano 2020, 14, 7617-7627, DOI: 10.1021/acsnano.0c03822
28. Qiu, G.; Gai, Z.; Tao, Y.; Schmitt, J.; Kullak-Ublick, G. A.; Wang, J. Dual-Functional Plasmonic Photothermal Biosensors for Highly Accurate Severe Acute Respiratory Syndrome Coronavirus 2 Detection. ACS Nano 2020, 14, 5268-5277, DOI: 10.1021/acsnano.0c02439
29. Huang, L.; Ding, L.; Zhou, J.; Chen, S.; Chen, F.; Zhao, C.; Xu, J.; Hu, W.; Ji, J.; Xu, H.; Liu, G. L. One-Step Rapid Quantification of SARS-CoV-2 Virus Particles via Low-Cost Nanoplasmonic Sensors in Generic Microplate Reader and Point-of-Care Device. Biosens. Bioelectron. 2021, 171, 112685, DOI: 10.1016/j.bios.2020.112685
30. Soler, M.; Estevez, M. C.; Cardenosa-Rubio, M.; Astua, A.; Lechuga, L. M. How Nanophotonic Label-Free Biosensors Can Contribute to Rapid and Massive Diagnostics of Respiratory Virus Infections: COVID-19 Case. ACS Sensors 2020, 5, 2663-2678, DOI: 10.1021/acssensors.0c01180
31. Ventura, B. D.; Cennamo, M.; Minopoli, A.; Campanile, R.; Censi, S. B.; Terracciano, D.; Portella, G.; Velotta, R. Colorimetric Test for Fast Detection of SARS-CoV-2 in Nasal and Throat Swabs. ACS Sensors 2020, 5, 3043-3048, DOI: 10.1021/acssensors.0c01742
32. Huang, C.; Wen, T.; Shi, F.-J.; Zeng, X.-Y.; Jiao, Y.-J. Rapid Detection of IgM Antibodies against the SARS-CoV-2 Virus via Colloidal Gold Nanoparticle-Based Lateral-Flow Assay. ACS Omega 2020, 5,12550-12556, DOI: 10.1021/acsomega.0c01554
33. Wen, T.; Huang, C.; Shi, F.-J.; Zeng, X.-Y.; Lu, T.; Ding, S.-N.; Jiao, Y.-J. Development of a Lateral Flow Immunoassay Strip for Rapid Detection of IgG Antibody against SARS-CoV-2 Virus. Analyst 2020, 145, 5345-5352, DOI: 10.1039/D0AN00629G
34. Chen, Z.; Zhang, Z.; Zhai, X.; Li, Y.; Lin, L.; Zhao, H.; Bian, L.; Li, P.; Yu, L.; Wu, Y.; Lin, G. Rapid and Sensitive Detection of anti-SARS-CoV-2 IgG, Using Lanthanide-Doped Nanoparticles-Based Lateral Flow Immunoassay. Anal. Chem. 2020, 92,7226-7231, DOI: 10.1021/acs.analchem.0c00784
35. Feng, M.; Chen, J.; Xun, J.; Dai, R.; Zhao, W.; Lu, H.; Xu, J.; Chen, L.; Sui, G.; Cheng, X. Development of a Sensitive Immunochromatographic Method Using Lanthanide Fluorescent Microsphere for Rapid Serodiagnosis of COVID-19. ACS Sensors 2020, 5, 2331-2337, DOI: 10.1021/acssensors.0c00927
36. Li, Z.; Yi, Y.; Luo, X.; Xiong, N.; Liu, Y.; Li, S.; Sun, R.; Wang, Y.; Hu, B.; Chen, W.; Zhang, Y.; Wang, J.; Huang, B.; Lin, Y.; Yang, J.; Cai, W.; Wang, X.; Cheng, J.; Chen, Z.; Sun, K. Development and Clinical Application of a Rapid IgM-IgG Combined Antibody Test for SARS-CoV-2 Infection Diagnosis. J. Med. Virol. 2020, 92, 1518-1524, DOI: 10.1002/jmv.25727
37. Fernandes, S. C.; Logounov, G. S.; Munro, J. B.; Mace, C. R. Comparison of Three Indirect Immunoassay Formats on a Common Paper-Based Microfluidic Device Architecture. Anal. Methods 2016, 8, 5204-5211, DOI: 10.1039/C6AY01558A
38. Kim, S.; Hao, Y.; Miller, E. A.; Tay, D. M. Y.; Yee, E.; Kongsuphol, P.; Jia, H.; Mcbee, M.; Preiser, P. R.; Sikes, H. D. Vertical Flow Cellulose-Based Assays for SARS-CoV-2 Antibody Detection in Human Serum. ACS Sensors 2021, 6, 1891-1898, DOI: 10.1021/acssensors.1c00235
39. Gasteiger, E.; Gattiker, A.; Hoogland, C.; Ivanyi, I.; Appel, R. D.; Bairoch, A. ExPASy: The Proteomics Server for In-Depth Protein Knowledge and Analysis. Nucleic Acids Res. 2003, 31, 3784-3788, DOI: 10.1093/nar/gkg563
40. Guo, X. Surface Plasmon Resonance Based Biosensor Technique: A Review. Journal of Biophotonics 2012, 5, 483-501, DOI: 10.1002/jbio.201200015
41. Tamada, K. Surface Plasmon Resonance. In Compendium of Surface and Interface Analysis; The Surface Science Society, Ed.; Springer Singapore: Singapore, 2018; pp 673-678.
42. Beeg, M.; Nobili, A.; Orsini, B.; Rogai, F.; Gilardi, D.; Fiorino, G.; Danese, S.; Salmona, M.; Garattini, S.; Gobbi, M. A Surface Plasmon Resonance-Based Assay to Measure Serum Concentrations of Therapeutic Antibodies and Anti-Drug Antibodies. Sci. Rep. 2019, 9, 2064, DOI: 10.1038/s41598-018-37950-4
43. Papeo, G.; Avanzi, N.; Bettoni, S.; Leone, A.; Paolucci, M.; Perego, R.; Quartieri, F.; Riccardi-Sirtori, F.; Thieffine, S.; Montagnoli, A.; Lupi, R. Insights into PARP Inhibitors' Selectivity Using Fluorescence Polarization and Surface Plasmon Resonance Binding Assays. J. Biomol. Screening 2014, 19, 1212-1219, DOI: 10.1177/1087057114538319
44. Moerke, N. J. Fluorescence Polarization (FP) Assays for Monitoring Peptide-Protein or Nucleic Acid-Protein Binding. Current Protocols in Chemical Biology 2009, 1, 1-15, DOI: 10.1002/9780470559277.ch090102
45. Rossi, A. M.; Taylor, C. W. Analysis of Protein-Ligand Interactions by Fluorescence Polarization. Nat. Protoc. 2011, 6, 365-387, DOI: 10.1038/nprot.2011.305
46. Liu, X.; Zhang, Q.; Knoll, W.; Liedberg, B.; Wang, Y. Rational Design of Functional Peptide-Gold Hybrid Nanomaterials for Molecular Interactions. Adv. Mater. 2020, 32, 2000866, DOI: 10.1002/adma.202000866
47. Aili, D.; Stevens, M. M. Bioresponsive Peptide-Inorganic Hybrid Nanomaterials. Chem. Soc. Rev. 2010, 39, 3358-3370, DOI: 10.1039/b919461 b
48. Liu, Y.; Franzen, S. Factors Determining the Efficacy of Nuclear Delivery of Antisense Oligonucleotides by Gold Nanoparticles. Bioconjugate Chem. 2008, 19,1009-1016, DOI: 10.1021/bc700421u
49. D'Agata, R.; Palladino, P.; Spoto, G. Streptavidin-Coated Gold Nanoparticles: Critical Role of Oligonucleotides on Stability and Fractal Aggregation. Beilstein J. Nanotechnol. 2017, 8, 1-11, DOI: 10.3762/bjnano.8.1
50. Warning, L. A.; Zhang, Q.; Baiyasi, R.; Landes, C. F.; Link, S. Nanoscale Surface-Induced Unfolding of Single Fibronectin Is Restricted by Serum Albumin Crowding. J. Phys. Chem. Lett. 2020, 11 (3), 1170-1177, DOI: 10.1021/acs.jpclett.9b03446
51. Jain, P. K.; Huang, W.; EI-Sayed, M. A. On the Universal Scaling Behavior of the Distance Decay of Plasmon Coupling in Metal Nanoparticle Pairs: A Plasmon Ruler Equation. Nano Lett. 2007, 7(7), 2080-2088, DOI: 10.1021/n1071008a
52. Li, N.; Su, X.; Lu, Y. Nanomaterial-Based Biosensors Using Dual Transducing Elements for Solution Phase Detection. Analyst 2015, 140 (9), 2916-2943, DOI: 10.1039/C4AN02376E
53. Andresen, H.; Mager, M.; Griefßner, M.; Charchar, P.; Todorova, N.; Bell, N.; Theocharidis, G.; Bertazzo, S.; Yarovsky, I.; Stevens, M. M. Single-Step Homogeneous Immunoassays Utilizing Epitope-Tagged Gold Nanoparticles: On the Mechanism, Feasibility, and Limitations. Chem. Mater. 2014, 26 (16), 4696-4704, DOI: 10.1021/cm500535p
54. Ma, H.; Zeng, W.; He, H.; Zhao, D.; Yang, Y.; Jiang, D.; Qi, P. Y.; He, W.; Zhao, C.; Yi, R.; Wang, X.; Wang, B.; Yang, Y. Y.; Kombe Kombe, A. J.; Ding, C.; Xie, J.; Gao, Y.; Cheng, L.; Li, Y.; Ma, X.; COVID-19 Diagnosis and Study of Serum SARS-CoV-2 Specific IgA, IgM and IgG by Chemiluminescence Immunoanalysis. medRxiv, 2020, DOI: 10.1101/2020.04.17.20064907 (accessed Mar. 22, 2021).
55. Galipeau, Y.; Greig, M.; Liu, G.; Driedger, M.; Langlois, M.-A. Humoral Responses and Serological Assays in SARS-CoV-2 Infections. Front. Immunol. 2020, 11, 610688, DOI: 10.3389/fimmu.2020.610688
56. Long, Q.-X.; Tang, X.-J.; Shi, Q.-L.; Li, Q.; Deng, H.-J.; Yuan, J.; Hu, J.-L.; Xu, W.; Zhang, Y.; Lv, F.-J.; Su, K.; Zhang, F.; Gong, J.; Wu, B.; Liu, X.-M.; Li, J.-J.; Qiu, J.-F.; Chen, J.; Huang, A.-L. Clinical and Immunological Assessment of Asymptomatic SARS-CoV-2 Infections. Nat. Med. 2020, 26,1200-1204, DOI: 10.1038/s41591-020-0965-6
57. Cervia, C.; Nilsson, J.; Zurbuchen, Y.; Valaperti, A.; Schreiner, J.; Wolfensberger, A.; Raeber, M. E.; Adamo, S.; Weigang, S.; Emmenegger, M.; Hasler, S.; Bosshard, P. P.; De Cecco, E.; Bachli, E.; Rudiger, A.; Stussi-Helbling, M.; Huber, L. C.; Zinkernagel, A. S.; Schaer, D. J.; Aguzzi, A. Systemic and Mucosal Antibody Responses Specific to SARS-CoV-2 during Mild versus Severe COVID-19. J. Allergy Clin. Immunol. 2021, 147,545-557, DOI: 10.1016/j.jaci.2020.10.040
58. Macmullan, M. A.; Ibrayeva, A.; Trettner, K.; Deming, L.; Das, S.; Tran, F.; Moreno, J. R.; Casian, J. G.; Chellamuthu, P.; Kraft, J.; Kozak, K.; Turner, F. E.; Slepnev, V. I.; Le Page, L. M. ELISA Detection of SARS-CoV-2 Antibodies in Saliva. Sci. Rep. 2020, 10, 20818, DOI: 10.1038/s41598-020-77555-4
59. Frens, G. Controlled Nucleation for the Regulation of the Particle Size in Monodisperse Gold Suspensions. Nature, Phys. Sci. 1973, 241, 20-22, DOI: 10.1038/physci241020a0

### APPLICATIONS

The present inventors have developed a nanosensor platform for rapid detection of SARS-CoV-2 antibodies by exploiting specific epitope-IgG interactions. This assay harnesses the innate ability of specific linear B-cell epitopes to selectively recognize SARS-CoV-2 IgG, together with the distance dependent plasmonic properties of AuNPs.

The present disclosure demonstrates the potential utility of epitope functionalized AuNPs in vaccine efficiency assessments, seroprevalence surveys, and targeted immunoassays to identify individuals with both late-stage and past SARS-CoV-2 infections, expanding the current toolbox available for SARS-CoV-2 serodiagnosis.

What is claimed is:

1. A nanosensor for detecting SARS-CoV-2 specific antibodies, comprising a metal nanoparticle functionalized with one or more B-cell linear peptide epitopes derived from the spike (S) protein or nucleocapsid (N) protein of SARS-CoV-2.

2. The nanosensor according to claim 1, wherein the one or more epitopes are conjugated to the nanoparticle surface either:
via a biotin-streptavidin interaction, or
via a thiol-metal interaction wherein the nanoparticle is coated with a spacer comprising at least 4 amino acids and at least one cysteine residue, optionally the spacer comprises the amino acid sequence CALNN.

3. The nanosensor according to claim 2, wherein the one or more epitopes are conjugated via a biotin-streptavidin interaction, for example wherein the metal nanoparticle is coated with streptavidin (SA) and the epitopes are biotinylated, or wherein the one or more epitopes are conjugated via a thiol-metal interaction, for example wherein the metal nanoparticle is coated with a thiolyated spacer and the epitopes are thiolyated.

4.-5. (canceled)

6. The nanosensor according to claim 1, wherein the metal is selected from the group consisting of gold, silver, and a combination of gold and silver, such as Au₅₀Ag₅₀.

7. The nanosensor according to claim 1, wherein the metal nanoparticle is a gold nanoparticle (AuNP).

8. The nanosensor according to claim 1, wherein the epitope is derived from the spike protein, optionally the epitope is derived from the S1 or S2 subunit of the spike protein, in particular the S2 subunit.

9. (canceled)

10. The nanosensor according to claim 1, wherein the epitope comprises an amino acid sequence selected from the group consisting of:
(S14P5)
(SEQ ID NO: 3)
TESNKKFLPFQQFGRDIA,
(S20P2)
(SEQ ID NO: 4)
GIAVEQDKNTQEVFAQVK,
(S21P2)
(SEQ ID NO: 5)
PSKPSKRSFIEDLLFNKV,
(N4P5)
(SEQ ID NO: 6)
NNAAIVLQLPQGTTLPKG,
and
an amino acid sequence at least 95% identical to one of the above, optionally the epitope comprises an amino acid sequence selected from the group consisting of:
(S14P5)
(SEQ ID NO: 3)
TESNKKFLPFQQFGRDIA,
(S20P2)
(SEQ ID NO: 4)
GIAVEQDKNTQEVFAQVK,
(S21P2)
(SEQ ID NO: 5)
PSKPSKRSFIEDLLFNKV,
and
an amino acid sequence at least 95% identical to one of the above, optionally wherein the epitope comprises an amino acid sequence selected from the group consisting of
(S14P5)
(SEQ ID NO: 3)
TESNKKFLPFQQFGRDIA,
(S21P2)
(SEQ ID NO: 5)
PSKPSKRSFIEDLLFNKV,
and
an amino acid sequence at least 95% identical to one of the above.

11.-12. (canceled)

13. The nanosensor according to claim 1, wherein the epitope comprises the amino acid sequence TESNKKFLPFQQFGRDIA (S14P5) (SEQ ID NO: 3) or an amino acid sequence at least 95% identical thereto.

14. The nanosensor according to claim 1, wherein the nanoparticle functionalized with at least two different epitopes, such as 2 to 20 different epitopes, for example 2, 3, 4, 5, 6, 7, 8, 9, 10, 11, 12, 13, 14, 15, 16, 17, 18, 19 or 20 different epitopes, optionally the nanoparticle is functionalized with two different epitopes, wherein at least one epitope is derived from the S1 subunit of the spike protein, and at least one other epitope is derived from the S2 subunit of the spike protein.

15.-16. (canceled)

17. The nanosensor according to claim 14, wherein the two different epitopes are TESNKKFLPFQQFGRDIA (S14P5) (SEQ ID NO: 3) or an amino acid sequence at least 95% identical thereto, and PSKPSKRSFIEDLLFNKV (S21P2) (SEQ ID NO: 5) or an amino acid sequence at least 95% identical thereto, or wherein the two different epitopes are TESNKKFLPFQQFGRDIA (S14P5) (SEQ ID NO: 3) or an amino acid sequence at least 95% identical thereto, and GIAVEQDKNTQEVFAQVK (S20P2) (SEQ ID NO: 4) or an amino acid sequence at least 95% identical thereto.

18. (canceled)

19. The nanosensor according to claim 1, wherein the nanoparticle is passivated, for example by coating the nanoparticle with a substance suitable for passivating the nanoparticle against biofluids (such as serum, blood, or plasma), for example by coating the nanoparticle with a polymer layer, optionally the nanoparticle is passivated by coating the nanoparticle with polyethylene glycol (PEG).

20. (canceled)

21. An in vitro assay or method for detecting the presence of SARS-CoV-2 specific antibodies in a sample, comprising a plurality of nanosensors, comprising a metal nanoparticle functionalized with one or more B-cell linear peptide epitopes derived from the spike (S) protein or nucleocapsid (N) protein of SARS-CoV-2, optionally further comprising one or more of the following: a stabilizer (such as Tween 20), a buffer (such as PBS), a control antibody (such as a SARS-CoV-2 antibody, a SARS-CoV antibody or a normal human IgG), a microplate, and combinations thereof, and optionally wherein the assay or method further comprises detecting the degree of nanoparticle aggregation.

22.-23. (canceled)

24. An in vitro method of diagnosing a subject as having a SARS-CoV-2 infection, comprising:
detecting the presence of SARS-CoV-2 specific antibodies in a sample from the subject by contacting the sample with a plurality of nanosensors comprising a metal nanoparticle functionalized with one or more B-cell linear peptide epitopes derived from the spike (S) protein or nucleocapsid (N) protein of SARS-CoV-2;
detecting the degree of nanoparticle aggregation in the sample;
comparing the degree of nanoparticle aggregation in the sample to the degree of nanoparticle aggregation in a control sample from a subject that does not have a SARS-CoV-2 infection; and
determining if the degree of nanoparticle aggregation is higher in the sample compared to the degree of nanoparticle aggregation in the control sample, thereby diagnosing the subject as having a SARS-CoV-2 infection.

25. A method of claim 24, further comprising administering a therapeutic agent, such as an anti-viral agent to the subject, optionally the therapeutic agent is selected from the group consisting of Paxlovid™ (nirmatrelvir and ritonavir), molnupiravir, fluvoxamine, sotrovimab, bebtelovimab, remdesivir and a combination thereof.

26. (canceled)

27. The method according to claim 24, wherein the degree of nanoparticle aggregation is proportional to the quantity of SARS-CoV-2 specific antibodies in the sample.

28. The method according to claim 24, wherein the range of SARS-CoV-2 specific antibodies that can be detected in the sample is about 3-1000 nM, such as about 3.2-1000 nM.

29. The method according to claim 24, wherein the nanoparticle aggregation results in a visible colour change, for example from red to purple, optionally wherein the degree of nanoparticle aggregation in a sample from a subject having a SARS-CoV-2 infection is more than the mean+3 standard deviations of the degree of nanoparticle aggregation in a control sample.

30. (canceled)

31. The method according to claim 24, wherein the degree of nanoparticle aggregation is detected by determining the % change in A₅₃₄ₙₘ, for example by measuring the absorbance using a spectrophotometer or microplate reader, optionally wherein a % change in A₅₃₄ₙₘ of about 15 to 25% or more, such as 20% or more indicates that the sample is from a subject having a SARS-CoV-2 infection.

32. (canceled)

33. The assay according to claim 21, wherein the sample is plasma or serum, in particular plasma.

34. The method according to claim 24, wherein the sample is plasma or serum, in particular plasma.


SEQUENCE LISTING (6 sequences)

SEQ ID NO: 1 - protein, 1273 aa, Artificial Sequence
  SARS-CoV-2 spike (S) protein amino acid sequence
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 419 aa, Artificial Sequence
  SARS-CoV-2 nucleocapsid (N) protein amino acid sequence
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 3 - protein, 18 aa, Artificial Sequence
  S14P5 peptide epitope amino acid sequence
TESNKKFLPFQQFGRDIA

SEQ ID NO: 4 - protein, 18 aa, Artificial Sequence
  S20P2 peptide epitope amino acid sequence
GIAVEQDKNTQEVFAQVK

SEQ ID NO: 5 - protein, 18 aa, Artificial Sequence
  S21P2 peptide epitope amino acid sequence
PSKPSKRSFIEDLLFNKV

SEQ ID NO: 6 - protein, 18 aa, Artificial Sequence
  N4P5 peptide epitope amino acid sequence
NNAAIVLQLPQGTTLPKG